SEQUENCE LISTING (60 sequences)

SEQ ID NO: 1 - DNA, 3536 bp, Homo sapiens
ggatcacgttggagggagcacacattgggagggtgggaacacaaggacaacgtagctcccactgaaacccacctgctgcccctacagcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccgacaagcagagctgctgctccctggggctgagtttcatcactatgtgggaggacctacggaaacccatgttgtaaggtccccacatccctaccagcttccacaggccttggccccccatgtggactttgtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacagtggggccgggtgttggtctgtctctggaagacaccagttccgccctaccttccctgcctccagcccctatgtcaccacagtgggaggcacatccttccaggaacctttcctcatcacaaatgaaattgttgactatatcagtggtggtggcttcagcaatgtgttcccacggccttcataccaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggcctctactccagtgtttggggggatcctatccttgatcaatgagcacaggatccttagtggccgcccccctcttggctttctcaacccaaggctctaccagcagcatggggcaggactctttgatgtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgcggtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcattgcgccccctgtgcctacctctagcattgtaacgaccacatagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccg

SEQ ID NO: 2 - protein, 320 aa, Homo sapiens
MRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 3 - DNA, 13 bp, Artificial Sequence
  Random primer H-AP38
aagcttccagtgc

SEQ ID NO: 4 - DNA, 16 bp, Artificial Sequence
  anchored oligo-dT primer
aagctttttttttttg

SEQ ID NO: 5 - DNA, 704 bp, Homo sapiens
atggacccgttcctggtgctgctgcactcggtgtcgtccagcctgtcgagcagcgagctgaccgagctcaagttcctatgcctcgggcgcgtgggcaagcgcaagctggagcgcgtgcagagcggcctagacctcttctccatgctgctggagcagaacgacctggagcccgggcacaccgagctcctgcgcgagctgctcgcctccctgcggcgccacgacctgctgcggcgcgtcgacgacttcgaggcgggggcggcggccggggccgcgcctggggaagaagacctgtgtgcggcatttaacgtcatatgtgataatgtggggaaagattggagaaggctggctcgtcagctcaaagtctcagacaccaagatcgacagcatcgaggacagatacccccgcaacctgacagagcgtgtgcgggagtcactgagaatctggaagaacacagagaaggagaacgcaacagtggcccacctggtgggggctctcaggtcctgccagatgaacctggtggctgacctggtacaagaggttcagcaggcccgtgacctccagaacaggagtggggccatgtccccgatgtcatggaactcagacgcatctacctccgaagcgtcctgatgggccgctgctttgcgctggtggaccacaggcatctacacagcctggactttggttctctccaggaaggtagccca

SEQ ID NO: 6 - protein, 208 aa, Homo sapiens
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 7 - DNA, 13 bp, Artificial Sequence
  Random primer H-AP32
aagcttcttgcaa

SEQ ID NO: 8 - DNA, 16 bp, Artificial Sequence
  anchored oligo-dT primer
aagctttttttttttg

SEQ ID NO: 9 - DNA, 630 bp, Homo sapiens
catggccaaagcctacgaccacctcttcaagttgctgctgatcggggactcgggggtgggcaagacttgtctgatcattcgctttgcagaggacaacttcaacaacacttacatctccaccatcggaattgatttcaagatccgcactgtggatatagaggggaagaagatcaaactacaagtctgggacacggctggccaagagcggttcaagacaataactactgcctactaccgtggagccatgggcattatcctagtatacgacatcacggatgagaaatctttcgagaatattcagaactggatgaaaagcatcaaggagaatgcctcggctggggtggagcgcctcttgctggggaacaaatgtgacatggaggccaagaggaaggtgcagaaggagcaggccgataagttggctcgagagcatggaatccgatttttcgaaactagtgctaaatccagtatgaatgtggatgaggcttttagttccctggcccgggacatcttgctcaagtcaggaggccggagatcaggaaacggcaacaagcctcccagtactgacctgaaaacttgtgacaagaagaacaccaacaagtgctccctgggctgaggaccctttcttgcctc

SEQ ID NO: 10 - protein, 203 aa, Homo sapiens
MAKAYDHLFKLLLIGDSGVGKTCLIIRFAEDNFNNTYISTIGIDFKIRTVDIEGKKIKLQVWDTAGQERFKTITTAYYRGAMGIILVYDITDEKSFENIQNWMKSIKENASAGVERLLLGNKCDMEAKRKVQKEQADKLAREHGIRFFETSAKSSMNVDEAFSSLARDILLKSGGRRSGNGNKPPSTDLKTCDKKNTNKCSLG

SEQ ID NO: 11 - DNA, 13 bp, Artificial Sequence
  Random primer H-AP35
aagcttcagggca

SEQ ID NO: 12 - DNA, 16 bp, Artificial Sequence
  anchored oligo-dT primer
aagcttttttttttta

SEQ ID NO: 13 - DNA, 718 bp, Homo sapiens
catggcttcagaagagctacagaaagatctagaagaggtaaaggtgttgctggaaaaggctactaggaaaagagtacgtgatgcccttacagctgaaaaatccaagattgagacagaaatcaagaacaagatgcaacagaaatcacagaagaaagcagaacttcttgataatgaaaaaccagctgctgtggttgctcccattacaacgggctatacggtgaaaatcagtaattatggatgggatcagtcagataagtttgtgaaaatctacattaccttaactggagttcatcaagttcccactgagaatgtgcaggtgcatttcacagagaggtcatttgatcttttggtaaagaatctaaatgggaagagttactccatgattgtgaacaatctcttgaaacccatctctgtggaaggcagttcaaaaaaagtcaagactgatacagttcttatattgtgtagaaagaaagtggaaaacacaaggtgggattacctgacccaggttgaaaaggagtgcaaagaaaaagagaagccctcctatgacactgaaacagatcctagtgagggattgatgaatgttctaaagaaaatttatgaagatggagacgatgatatgaagcgaaccattaataaagcctgggtggaatcaagagagaagcaagccaaaggagacacggaattttgagactttaaagtcgttttgggaactgtgatg

SEQ ID NO: 14 - protein, 228 aa, Homo sapiens
MASEELQKDLEEVKVLLEKATRKRVRDALTAEKSKIETEIKNKMQQKSQKKAELLDNEKPAAVVAPITTGYTVKISNYGWDQSDKFVKIYITLTGVHQVPTENVQVHFTERSFDLLVKNLNGKSYSMIVNNLLKPISVEGSSKKVKTDTVLILCRKKVENTRWDYLTQVEKECKEKEKPSYDTETDPSEGLMNVLKKIYEDGDDDMKRTINKAWVESREKQAKGDTEF

SEQ ID NO: 15 - DNA, 13 bp, Artificial Sequence
  Random primer H-AP34
aagcttcagcagc

SEQ ID NO: 16 - DNA, 16 bp, Artificial Sequence
  anchored oligo-dT primer
aagctttttttttttg

SEQ ID NO: 17 - DNA, 1144 bp, Homo sapiens
ggaacggccgaaaagatggcggtcttggcacctctaattgctctcgtgtattcggtgccgcgactttcacgatggctcgcccaaccttactaccttctgtcggccctgctctctgctgccttcctactcgtgaggaaactgccgccgctctgccacggtctgcccacccaacgcgaagacggtaacccgtgtgactttgactggagagaagtggagatcctgatgtttctcagtgccattgtgatgatgaagaaccgcagatccatcactgtggagcaacatataggcaacattttcatgtttagtaaagtggccaacacaattcttttcttccgcttggatattcgcatgggcctactttacatcacactctgcatagtgttcctgatgacgtgcaaaccccccctatatatgggccctgagtatatcaagtacttcaatgataaaaccattgatgaggaactagaacgggacaagagggtcacttggattgtggagttctttgccaattggtctaatgactgccaatcatttgcccctatctatgctgacctctcccttaaatacaactgtacagggctaaattttgggaaggtggatgttggacgctatactgatgttagtacgcggtacaaagtgagcacatcacccctcaccaagcaactccctaccctgatcctgttccaaggtggcaagaggcaatgcggcggccacagattgacaagaaaggacgggctgtctcatggaccttctctgaggagaatgtgatccgagaatttaacttaaatgagctataccagcgggccaagaaactatcaaaggctggagacaatatccctgaggagcagcctgtggcttcaacccccaccacagtgtcagatggggaaaacaagaaggataaataagatcctcactttggcagtgcttcctctcctgtcaattccaggctctttccataaccacaagcctgaggctgcagccttttatttatgttttccctttggctgtgactgggtggggcagcatgcagcttctgattttaaagaggcatctagggaattgtcaggcaccctacaggaaggcctgccatgctgtggccaactgtttcactggagcaagaaagagatctcataggacggagggg

SEQ ID NO: 18 - protein, 250 aa, Homo sapiens
MAVLAPLIALVYSVPRLSRWLAQPYYLLSALLSAAFLLVRKLPPLCHGLPTQREDGNPCDFDWREVEILMFLSAIVMMKNRRSITVEQHIGNIFMFSKVANTILFFRLDIRMGLLYITLCIVFLMTCKPPLYMGPEYIKYFNDKTIDEELERDKRVTWIVEFFANWSNDCQSFAPIYADLSLKYNCTGLNFGKVDVGRYTDVSTRYKVSTSPLTKQLPTLILFQGGKRQCGGHRLTRKDGLSHGPSLRRM

SEQ ID NO: 19 - DNA, 13 bp, Artificial Sequence
  Random primer H-AP36
aagcttcgacgct

SEQ ID NO: 20 - DNA, 16 bp, Artificial Sequence
  anchored oligo-dT primer
aagcttttttttttta

SEQ ID NO: 21 - DNA, 3020 bp, Homo sapiens
cgtcggggctggagccggagcgcgccgggcgctgggcgcagcgagcgagagcgcggcggccgcgggctccggcgagggacagacgcaccgatcgccggagggacagacacacgaccacgcggcgccaccgcccacgcctccacccaccggcgcccaagtcctccccgcgccgcctcctctgtatggcacaaactttcctcccgggacggaacacgctgcctcagggagcccgcgaccgcgccttctcctccgccggtcccatacgctgctgaaatgggttgcggattgaacaagttagagaaacgtgatgaaaaacggcctgggaatatttattcaactttgaagaggcctcaggtggaaaccaagatagatgtgtcctatgaataccgcttcctggagttcacgactctgagtgctgcggagctccctgggtcctcagcagtgaggctggcctccctgcgtgacctgcccgcccagctcctggagctgtaccagcagggcttctcgctggcggccctgcaccccttcgtgcagcccacccatgagcgggagaagacgcccctggagcacatctttagagccatcctgatcaagaaaaccgacagatctcagaaaactgatcttcacaatgaaggctacatcttggaattagattgctgttcctccttagaccacccgacagaccagaaactcatcccagagttcattaagaagatccaggaggctgcaagccagggcctgaaattcgttggtgttatacctcagtaccattcctctgtgaactcggcaggcagcagtgctccggtgtctactgccaacagcaccgaggatgccagagatgcaaaaaacgcacgtggggatcacgcgtcactggagaatgagaaaccggggactggggatgtgtgcagtgctccggctgggagaaaccaaagcccagagcccagctcaggccccagaggggaggtgcccctcgccaagcagcccagctcaccctccggagagggagatggtggagaactttcaccacagggggtgagcaagacactggatggaccggagagcaaccccttggaggtgcatgaagagccactctcagggaaaatggagatcttcacccttttcaacaaaccgaagagccatcagaagtgccggcaatactaccctgtcaccattcctctccatgtctccaagaatggccagacagtgagcggtttggacgccaactggttagagcacatgagcgaccacttccggaaaggaggcatgctggtgaacgcagtcttctaccttggaatagtgaatgattccttacatggcttgacagatggagtattcatctttgaagctgtttccacagaagatagcaaaaccatacagggctatgatgctattgtggttgaacaatggacagtcctggaaggtgtcgaagtgcagacagactacgtgcccctgctgaactcgctggcggcctatggctggcagctcacctgtgtgctaccaactcccgtcgtcaagactaccagcgaggggagtatatccaccaagcagattgtctttcttcagagaccgtgtctacctcagaaaatcaagaagaaggaatcgaagtttcagtggcgattctccagagaagaaatgcacaacaggcagatgaggaaatcaaaaggtaaactcagtgccagagacaaacaacaagcagaagaaaatgagaagaacttagaagaccagtcttccaaagctggagacatgggaaactgtgtttcgggacagcagcaggagggtggagtctccgaggagatgaagggccctgtccaagaggacaagggagaacagctgtcccctggtggcctgctgtgtggggtgggtgtggagggtgaggctgtgcagaatggtcctgccagccacagcagggccctggtggggatttgcactgggcactccaatcctggagaggatgccagggacggggatgctgaggaagtcagagagcttggtacggttgaagaaaactgagtcttgggcaatttgtgctaaaactaggtgagttgccaaacccaaggcatcttaccaacagctggtttgggggctggtttccctggtgttgtgtgttacctaccctttggcttggcttgacctctccttgtgagctcacctgagccctcccagggccaggttcctgacagtgttggtttttgcacatccactggaaaggtgtcattaatgacccagtgttagaatgcaagaggtcaggttattctagccctcatggctgaaggcccagtcctggctccaccactcctccagccagagggtctggaccatccagtgcctgtcctcgccacagggcctccagggagcattcgggtcaaatccatggacaccctgggctacaaaccaaggctgctgttcatcccacatcgtgtggggcagtgtccatcccctgcagctacttggtgacttaacaactccaggagccctgtcagctgccctcctccacctaaaccccttcgactcttctgctttgacaaagaaaatgacattggggaggggaggtgctccgcctcccagcttttctcaaaatagtcctatagatactggtaatctggaaatgaagaagtaattctgtctctgcacctacttttgcagaatgttcaaggaagtattctgtgttagtattaatgccaaaaagttgtttttaaaggttttgtactcagcacatcatacaaaccacattacttctgtcacttcagggcatcgggactggctggcgcccttgttatgtgctattttaatcagtgtaacattggtcaagttgttacccatgtatgctgtgtttatcatgtgtatatcgtctagaaagtattaaggctttaggtagatgcaactggcgaaccttggagagggaatgctgattgtcttgaccaaacccacagcctgtctcttctcttgtttagttacttacggcaataaatcatctatgagttaaaaaaaaaaaaaaaaaaaaaaaaaaaaaaaaaaaaaaa

SEQ ID NO: 22 - protein, 578 aa, Homo sapiens [sequence omitted]

SEQ ID NO: 23 - DNA, 13 bp, Artificial Sequence
  Random primer H-AP35
aagcttcagggca

SEQ ID NO: 24 - DNA, 16 bp, Artificial Sequence
  anchored oligo-dT primer
aagcttttttttttta

SEQ ID NO: 25 - DNA, 2367 bp, Homo sapiens
cggcgatcgcgggctgaggctgctcccggacaagggcaacgagcgtttcgtttggacttctcgacttgagtgcccgcctccttcgccgccgcctctgcagtcctcagcgcagttatgcccagttcttcccgctgtggggacacgaccacggaggaatccttgcttcagggactcgggaccctgctggaccccttcctcgggtttaggggatgtggggaccaggagaaagtcaggatccctaagagtcttccctgcctggatggatgagtggcttcttctccacctagattctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacattatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagttaaaaaaaaaaaaaaa

SEQ ID NO: 26 - protein, 640 aa, Homo sapiens
MESVVRRCPFLSRVPQAFLQKAGKSLLFYAQNCPKMMEVGAKPAPRALSTAAVHYQQIKETPPASEKDKTAKAKVQQTPDGSQQSPDGTQLPSGHPLPATSQGTASKCPFLAAQMNQRGSSVFCKASLELQEDVQEMNAVRKEVAETSAGPSVVSVKTDGGDPSGLLKNFQDIMQKQRPERVSHLLQDNLPKSVSTFQYDRFFEKKIDEKKNDHTYRVFKTVNRRAHIFPMADDYSDSLITKKQVSVWCSNDYLGMSRHPRVCGAVMDTLKQHGAGAGGTRNISGTSKFHVDLERELADLHGKDAALLFSSCFVANDSTLFTLAKMMPGCEIYSDSGNHASMIQGIRNSRVPKYIFRHNDVSHLRELLQRSDPSVPKIVAFETVHSMDGAVCPLEELCDVAHEFGAITFVDEVHAVGLYGARGGGIGDRDGVMPKMDIISGTLGKAFGCVGGYIASTSSLIDTVRSYAAGFIFTTSLPPMLLAGALESVRILKSAEGRVLRRQHQRNVKLMRQMLMDAGLPVVHCPSHIIPVRVADAAKNTEVCDELMSRHNIYVQAINYPTVPRGEELLRIAPTPHHTPQMMNYFLENLLVTWKQVGLELKPHSSAECNFCRRPLHFEVMSEREKSYFSGLSKLVSAQA

SEQ ID NO: 27 - DNA, 13 bp, Artificial Sequence
  Random primer H-AP31
aagcttggtgaac

SEQ ID NO: 28 - DNA, 16 bp, Artificial Sequence
  anchored oligo-dT primer
aagcttttttttttta

SEQ ID NO: 29 - DNA, 1564 bp, Homo sapiens
gcttctttcacactttagttgggagctgcgcgccgcgctcagttactggagagctggccgcgcgccgccgcctcccgcacgcttgcacgcgggcccggcttcggggttttgggttcttactccaagcggcggggaggagggggagccccggacacactgtggggaggaggaggaagaagaggaggagggaggaagaaaaaagacgaggaggacaggggcggggggcgggaggcttgccaccttcagcccccccgcgaacgcccaaggtgcacacatcttgaccaactcagcagcaaggtggattttctttgtgtttaaagaaaaaaaatgtccctgtgtctgtagagatgatttgcagttcagcccggctgaagctgaccgaatgagactatgggctgtgcctccgccaagcatgttgccactgttcaaaatgaagaggaagcccagaaagggaaaaactaccagaacggagatgtgtttggcgatgagtataggatcaaaccagtggaagaggtcaaatacatgaaaaatggggcagaagaagagcagaaaatagcagccaggaaccaagaaaacttggaaaaaagtgccagctcaaatgtaagacttaaaactaataaagaggttccgggattagttcatcaacccagagcaaacatgcacatctctgaaagccaacaagaattcttcagaatgctggatgaaaaaattgaaaagggtcgggattactgttcggaagaagaggatatcacatagcaccaattttaccactcaaaccaggagctactactgtgtaaataggttacaccccagttgaaatctttgcaaaggtcggttctattcagcgaacagcactatagcaaaagaagatcgttccatattgtacgccccattaaattacagtgtttcttaatgaacttgcaaaggaatattgctaaaaacaaacaaaaaaaactgttatcgaactttctttgttgctgctagttaaaacttgttgcaacttttcacttctcttgtgtccaggtatgcagcaaaattctgcaatttcaccttaaagatactgttggttttacagatgctctccaacctattttctataagatgaggtagtggtgaactcagataacaaacttctcttctaaactggttctgcttctaagacaagcatctcctgccctctctccttcctccccatctctcgcacgcagtctagagatggactgagccttgcttctcactggcagtgttgagctttggagatgggatggttgctatgccaagccttgtttctctgctcagaagaagtagagaagctattatcaattaaaagcatgctgtgatgtgactcctggaagtgacgtaggagtgagtggcaggttgtttgatttaataggtatcttaatcaagaattaagcttgcaacattggctttgctcagatgcagatggaagtgtgatcacaatcattttgaatccctcttcctcacttttttttctaagaaaataaacattttactgtttttatggaaaaaaaaaaaaaaaaaaaaaaaaaaaaaaaaaaaaaaa

SEQ ID NO: 30 - protein, 121 aa, Homo sapiens
MGCASAKHVATVQNEEEAQKGKNYQNGDVFGDEYRIKPVEEVKYMKNGAEEEQKIAARNQENLEKSASSNVRLKTNKEVPGLVHQPRANMHISESQQEFFRMLDEKIEKGRDYCSEEEDIT

SEQ ID NO: 31 - DNA, 13 bp, Artificial Sequence
  Random primer H-AP6
aagcttgcaccat

SEQ ID NO: 32 - DNA, 16 bp, Artificial Sequence
  anchored oligo-dT primer
aagctttttttttttg

SEQ ID NO: 33 - DNA, 1821 bp, Homo sapiens
ggctctagagggggtactatagggtaaatacgggccctatttcaaagatttttaggggaattaattctaggacgatgggcatgaaactgtggtttgctccacagatttcagagcattgaccgtagtatacccccggtcgtgtagcggtgaaagtggtttggtttagacgtccgggaattgcatctgtttttaagcctaatgtggggacagctcatgagtgcaagacgtcttgtgatgtaattattatacgaatgggggcttcaatcgggagtactactcgattgtcaacgtcaaggagtcgcaggtcgcctggttctaggaataatgggggaagtatgtaggagttgaagattagtccgccgtagtcggtgtactcgtaggttcagtaccattggtggccaattgatttgatggtaagggagggatcgttgacctcgtctgttatgtaaaggatgcgtagggatgggagggcgatgaggactaggatgatggcgggcaggatagttcagacggtttctatttcctgagcgtctgagatgttagtattagttagttttgttgtgagtgttaggaaaagggcatacaggactaggaagcagataaggaaaatgattatgagggcgtgatcatgaaaggtgataagctccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtacccaggtgttgtctttgaggtcttgggatgaatcagaaatctatccaggctatcttccagattccttaagtgccgttgttcagttctaatcacactaatcaaaaagaaacgagtatttgtatttattaaactcattagtttgggcagtatactaaggtgtggctgtcttggattcagatagaactaagggttcccgactctgaatccagagtctgagttaaatgtttccaatggttcagtctagctttcacagtttttatgaataaaaggcattaaaggctaaaaaaaaaaaaaaaaaaaaaaaaaaaaaaaaaaaaa

SEQ ID NO: 34 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 35 - DNA, 13 bp, Artificial Sequence
  Random primer H-AP6
aagcttgcaccat

SEQ ID NO: 36 - DNA, 16 bp, Artificial Sequence
  anchored oligo-dT primer
aagctttttttttttg

SEQ ID NO: 37 - DNA, 3116 bp, Homo sapiens
ccagcaccagcacccttgcccttttccatcaggggttcagcctagggtccccgctggtgggcggctcccgagtcttggagaagagcacgagaacctagaccgcccccgaagtgcggagaccccctgggcaggctgaaagatggcggcggcgtctgtctctgcggcttctggttctcacttgtcgaacagctttgctgagccatcaaggtctaatggaagcatggttcggcattcttcatctccatatgtagtatatccttcggataagcctttccttaatagtgatctacgacgctccccaagtaagcctacacttgcctatccagaaagcaacagcagagccatattttctgctcttaagaatcttcaagataagattcgacgcttggaacttgagaggattcaggcagaagaaagtgtgaaaaccttgtctagagaaacaattgaatataagaaagtactggatgaacagatacaagaaagggagaattcaaagaatgaggaatcaaagcacaatcaagaactgacatctcagttgttagctgcagaaaataaatgcaatctattagaaaaacaattggaatacatgcgaaatatgataaagcatgccgaaatggagaggacatctgtcttagagaaacaagtttccctagaaagagaacgacaacatgatcaaacacatgttcagagccaacttgaaaaattggatcttcttgaacaggagtataacaaacttaccacaatgcaggcccttgcagaaaaaaaaatgcaagagttggaagcaaaactccatgaagaagaacaggaaaggaaacgcatgcaagctaaggcagctgagttgcagactggtctagaaacaaatagacttatctttgaagataaggcaactccgtgtgttcccaatgcaagaagaattaaaaaaaagaagtcaaaaccaccagaaaagaaaagttctaggaactattttggtgcacaaccacattatagattatgcttgggtgatatgccatttgtagctgggaagtccacaagccctagccatgccgtggtagccaatgttcagcttgtcttgcatctaatgaagcaacacagtaaagctttgtgcaatgatcgagtcatcaacagtattcctttggcaaagcaagtatcttcacgaggtggtaaaagtaagaagttgtcagtaacacctccctcctccaacggtattaatgaggagttgtcagaagtcttacagactttacaggatgaatttgggcaaatgagctttgatcaccagcagcttgcaaaacttatccaggagtcgccaaccgttgaactgaaagacaagttggagtgtgaattggaggcattagtgggaaggatggaagcaaaagccaaccaaataactaaagttcgaaaataccaagcccagctggagaaacagaagttagagaagcagaagaaggaattaaaagctaccaaaaagactcttgatgaagaaagaaacagcagcagccgttctggaatcacagggaccacaaataagaaagattttatgaaactgagacctggagaaaaaaggagaaaaaatcttcagttattgaaggacatgcaaagcatacagaattcattacaaagcagtagtttgtgttgggattactgactcataaccaggtcagaaattttattcagataatctgtacctcatcaatcagatgatgacaatttacttcccaggtctcatactcacttatgttggaattaattaatagcaggtgttaaaggacccaggcttcattacacaggcttttcatgtatgcaggatgactcaatgttaaagcatttaaatggaaaccaggggagttttaaagcccgagaaaccacacataatcttttgttgagatgagtttgctgtactgacgctgcactttgtaaacagattaccagttttttacttgtgggtgtgattttttaaaatagttctttatatataattaaaattaggtttaaattttcaaaatatgagactatgctataggcagtgcttgcttgaaaagtctcatttttaaatctccctggcatgaggtgcccacttcccctttccaaagcaaccattaaacatactttgtttctactattggtggagtttttctatatttaaaaatacatatatatttcagaggatttttattttgctttttggcatttcagactttgatcagtgttaagtgcacttgtattgctttttaatctgttaattttttaaagacccaagcagtcattttgagttatatctataaaaattataaaaggatttttgaaagtataaacaaattgtcagtgaaataaatgagattttggaataaagtgagaatgggagaagggatatgttgtgagcatataccttcacagttcttaatacctgttttgtaatcatgatattcagtcaaggcattatggtttttaatcttgaaacttagagaaccctttgaatatttgcttttactggtgtacagtatgagtggaatataaactgtacacataattatcatgttgatataaatcataatttcaactagatcaagacatgttaaccttttataaatttaaagtcaataaagcacctttttaaaggaaacactgcaactttccttaacagcctgttaataaaggctttgtgacaagctctcaaaaaatgcatttctaaataggaggacatcaatgtattgatgaagagaaaaaactagtacttagttgccacactcatgcttacatagaaagagagcccaagaatattagatttcctcatgatacaagatactacagtaacaggctttaatttaggatccttaagattttggggtattatttgtgactctcctgaaattgtaaacttgtgcttctgtgtccagttttctaatgagtaggttcgtagcttgattgaattaataattgtgagcccatagacacaagggaagtgagaaacagtgctctggtgacatgataaatatatgtgtcaaccaccatttcagctattaaaaactcctgttatctccttgtttgaatttcaggtcattaaattgtataaccatcattttaaaaaaaaaaaaaaaaaaaaaaaaaaaaaaaaaaaaaaa

SEQ ID NO: 38 - protein, 500 aa, Homo sapiens
MAAASVSAASGSHLSNSFAEPSRSNGSMVRHSSSPYVVYPSDKPFLNSDLRRSPSKPTLAYPESNSRAIFSALKNLQDKIRRLELERIQAEESVKTLSRETIEYKKVLDEQIQERENSKNEESKHNQELTSQLLAAENKCNLLEKQLEYMRNMIKHAEMERTSVLEKQVSLERERQHDQTHVQSQLEKLDLLEQEYNKLTTMQALAEKKMQELEAKLHEEEQERKRMQAKAAELQTGLETNRLIFEDKATPCVPNARRIKKKKSKPPEKKSSRNYFGAQPHYRLCLGDMPFVAGKSTSPSHAVVANVQLVLHLMKQHSKALCNDRVINSIPLAKQVSSRGGKSKKLSVTPPSSNGINEELSEVLQTLQDEFGQMSFDHQQLAKLIQESPTVELKDKLECELEALVGRMEAKANQITKVRKYQAQLEKQKLEKQKKELKATKKTLDEERNSSSRSGITGTTNKKDFMKLRPGEKRRKNLQLLKDMQSIQNSLQSSSLCWDY

SEQ ID NO: 39 - DNA, 13 bp, Artificial Sequence
  H-AP36 primer
aagcttcgacgct

SEQ ID NO: 40 - DNA, 16 bp, Artificial Sequence
  anchored oligo-dT primer
aagcttttttttttta

SEQ ID NO: 41 - DNA, 1451 bp, Homo sapiens
cgttccagtgttccagcttctgcgagatgaccctcaaggcgagcgagggcgagagtgggggcagcatgcacacggcgctctccgacctctacctggagcatttgctgcagaagcgtagtcggccagaggctgtatcgcatccattgaatactgtgaccgaggacatgtacaccaacgggtctcctgccccaggtagccctgcccaggtcaagggacaggaggtgcggaaagtgcgactcatacagtttgagaaggtcacagaagagcccatgggaatcactctgaagctgaatgaaaaacagtcctgtacggtggccagaattcttcatggtggcatgatccatagacaaggctcccttcacgtgggggatgagatcctagaaatcaatggcacaaatgtgacaaatcattcagtggatcagctgcagaaggcgatgaaagaaaccaaaggaatgatctcattaaaagtaattcccaaccagcaaagccgtcttcctgcactacagatgttcatgagagcgcagtttgactatgatcccaaaaaggacaatctgatcccttgcaaggaggcgggactgaagtttgctactggggacattatccagattatcaacaaggatgacagcaattggtggcagggacgggtggaaggctcctccaaggagtcagcaggattgatcccttcccctgagctgcaggaatggcgagtggcaagtatggctcagtcagctcctagcgaagccccgagctgcagtccctttgggaagaagaagaagtacaaagacaaatatctggccaagcacagctcgatttttgatcagttggatgttgtttcctacgaggaagtcgttcggctccctgcattcaagaggaagaccctggtgctgatcggagccagtggggtgggtcgcagccacattaagaatgccctgctcagccagaatccggagaagtttgtgtaccctgtcccatatacaacacggccgccaaggaagagtgaggaagatgggaaggagtaccactttatctcaacggaggagatgacgaggaacatctctgccaatgagttcttggagtttggcagctaccaaggcaacatgtttggcaccaaatttgaaacagtgcaccagatccataagcagaacaagattgccatccttgacattgagccccagaccctgaaaattgttcggacagcagaactttcgcctttcattgtgttcattgcacctactgaccagggcactcagacagaagccctgcagcagctgcagaaggactctgaggccatccgcagccagtacgctcactactttgacctctcactggtcaataatggtgttgatgaaacccttaagaaattacaagaagccttcgaccaagcgtgcagttctccacagtgggtgcctgtctcctgggtttactaagcttgtagaatgggggaacccact

SEQ ID NO: 42 - protein, 466 aa, Homo sapiens
MTLKASEGESGGSMHTALSDLYLEHLLQKRSRPEAVSHPLNTVTEDMYTNGSPAPGSPAQVKGQEVRKVRLIQFEKVTEEPMGITLKLNEKQSCTVARILHGGMIHRQGSLHVGDEILEINGTNVTNHSVDQLQKAMKETKGMISLKVIPNQQSRLPALQMFMRAQFDYDPKKDNLIPCKEAGLKFATGDIIQIINKDDSNWWQGRVEGSSKESAGLIPSPELQEWRVASMAQSAPSEAPSCSPFGKKKKYKDKYLAKHSSIFDQLDVVSYEEVVRLPAFKRKTLVLIGASGVGRSHIKNALLSQNPEKFVYPVPYTTRPPRKSEEDGKEYHFISTEEMTRNISANEFLEFGSYQGNMFGTKFETVHQIHKQNKIAILDIEPQTLKIVRTAELSPFIVFIAPTDQGTQTEALQQLQKDSEAIRSQYAHYFDLSLVNNGVDETLKKLQEAFDQACSSPQWVPVSWVY

SEQ ID NO: 43 - DNA, 13 bp, Artificial Sequence
  H-AP21 primer
aagctttctctgg

SEQ ID NO: 44 - DNA, 16 bp, Artificial Sequence
  anchored oligo-dT primer
aagctttttttttttg

SEQ ID NO: 45 - DNA, 1079 bp, Homo sapiens
tcaccttgcaatgccaataaccatcaatgatgttcccccttgtatctctcaattacttgataatgaggagagttgggacttcaacatctttgaattggaagccattacgcataaaaggccattggtttatctgggcttaaaggtcttctctcggtttggagtatgtgagtttttaaactgttctgaaaccactcttcgggcctggttccaagtgatcgaagccaactaccactcttccaatgcctaccacaactccacccatgctgccgacgtcctgcacgccaccgctttctttcttggaaaggaaagagtaaagggaagcctcgatcagttggatgaggtggcagccctcattgctgccacagtccatgacgtggatcacccgggaaggaccaactctttcctctgcaatgcaggcagtgagcttgctgtgctctacaatgacactgctgttctggagagtcaccacaccgccctggccttccagctcacggtcaaggacaccaaatgcaacattttcaagaatattgacaggaaccattatcgaacgctgcgccaggctattattgacatggttttggcaacagagatgacaaaacactttgaacatgtgaataagtttgtgaacagcatcaacaagccaatggcagctgagattgaaggcagcgactgtgaatgcaaccctgctgggaagaacttccctgaaaaccaaatcctgatcaaacgcatgatgattaagtgtgctgacgtggccaacccatgccgccccttggacctgtgcattgaatgggctgggaggatctctgaggagtattttgcacagactgatgaagagaagagacagggactacctgtggtgatgccagtgtttgaccggaatacctgtagcatccccaagtctcagatctctttcattgactacttcataacagacatgtttgatgcttgggatgcctttgcacatctgccagccctgatgcaacatttggctgacaactacaaacactggaagacactagatgacctaaagtgcaaaagtttgaggcttccatctgacagctaaagccaagccacagagg

SEQ ID NO: 46 - protein, 350 aa, Homo sapiens
MPITINDVPPCISQLLDNEESWDFNIFELEAITHKRPLVYLGLKVFSRFGVCEFLNCSETTLRAWFQVIEANYHSSNAYHNSTHAADVLHATAFFLGKERVKGSLDQLDEVAALIAATVHDVDHPGRTNSFLCNAGSELAVLYNDTAVLESHHTALAFQLTVKDTKCNIFKNIDRNHYRTLRQAIIDMVLATEMTKHFEHVNKFVNSINKPMAAEIEGSDCECNPAGKNFPENQILIKRMMIKCADVANPCRPLDLCIEWAGRISEEYFAQTDEEKRQGLPVVMPVFDRNTCSIPKSQISFIDYFITDMFDAWDAFAHLPALMQHLADNYKHWKTLDDLKCKSLRLPSDS

SEQ ID NO: 47 - DNA, 13 bp, Artificial Sequence
  H-AP24 primer
aagcttcactagc

SEQ ID NO: 48 - DNA, 16 bp, Artificial Sequence
  anchored oligo-dT primer
aagcttttttttttta

SEQ ID NO: 49 - DNA, 327 bp, Homo sapiens
ggaggaaggtgggtctgaatctagcaccttgacggaactagagacagccatgggcatgatcatggacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggaga

SEQ ID NO: 50 - protein, 88 aa, Homo sapiens [sequence omitted]

SEQ ID NO: 51 - DNA, 13 bp, Artificial Sequence
  H-AP12 primer
aagcttgagtgct

SEQ ID NO: 52 - DNA, 16 bp, Artificial Sequence
  anchored oligo-dT primer
aagctttttttttttg

SEQ ID NO: 53 - DNA, 822 bp, Homo sapiens
tttttgatgttccccaattccccaccagccaatccaagcattttctcctgggacccgctccatgttcagggtctaagacccagaatgagtttctccagccaggtggccactttatggaaaggatgcagggtcccccagacccctcctctagcccagcagcggctggggtctggctcctttcagagacactacacttcccgtgcagactcagatgccaactcaaagtgccttagcccaggttcctgggcttctgctgccccctcaggaccccagctaacctcagaacaactaactcatgtggactcggaaggacgggcagctatggtagatgtgggcaggaagccagacacagagcgggtggctgtggcttcagccgtggtcctcctgggaccggtagccttcaagcttgtccagcagaaccagctcaagaaaggagatgccctagtggtggcccagctggctggagtccaggcagccaaggtgaccagccagctgatccctctgtgccaccacgtggccctgagccacatccaggtgcagctggagctggacagcacacgccatgccgtgaagatccaggcatcttgccgggctcggggccccaccggggtggagatggaggccctgacctctgctgcagtggccgccctcaccctgtatgacatgtgcaaggctgtcagcagggacatcgtgttggaggagatcaagctcattagcaagactggtggtcagcggggggacttccatcgggcttagcacctgcccttctcacccatggcccacccaggcctggagctgggatgcaatgtaggctgagggaaa

SEQ ID NO: 54 - protein, 249 aa, Homo sapiens
MFPNSPPANPSIFSWDPLHVQGLRPRMSFSSQVATLWKGCRVPQTPPLAQQRLGSGSFQRHYTSRADSDANSKCLSPGSWASAAPSGPQLTSEQLTHVDSEGRAAMVDVGRKPDTERVAVASAVVLLGPVAFKLVQQNQLKKGDALVVAQLAGVQAAKVTSQLIPLCHHVALSHIQVQLELDSTRHAVKIQASCRARGPTGVEMEALTSAAVAALTLYDMCKAVSRDIVLEEIKLISKTGGQRGDFHRA

SEQ ID NO: 55 - DNA, 13 bp, Artificial Sequence
  H-AP13 primer
aagcttcggcata

SEQ ID NO: 56 - DNA, 16 bp, Artificial Sequence
  anchored oligo-dT primer
aagcttttttttttta

SEQ ID NO: 57 - DNA, 2049 bp, Homo sapiens
gccgccgccgccgccacctctgagcagccggctgggagcgagagccgacagctagtctgcaagccaccgctgtcgccatggggagccgcgtctcgcgggaagacttcgagtgggtctacaccgaccagccgcacgccgaccggcgccgggagatcctggcaaagtatccagagataaagtccttgatgaaacctgatcccaatttgatatggattataattatgatggttctcacccagttgggtgcattttacatagtaaaagacttggactggaaatgggtcatatttggggcctatgcgtttggcagttgcattaaccactcaatgactctggctattcatgagattgcccacaatgctgcctttggcaactgcaaagcaatgtggaatcgctggtttggaatgtttgctaatcttcctattgggattccatattcaatttcctttaagaggtatcacatggatcatcatcggtaccttggagctgatggcgtcgatgtagatattcctaccgattttgagggctggttcttctgtaccgctttcagaaagtttatatgggttattcttcagcctctcttttatgcctttcgacctctgttcatcaaccccaaaccaattacgtatctggaagttatcaataccgtggcacaggtcacttttgacattttaatttattactttttgggaattaaatccttagtctacatgttggcagcatctttacttggcctgggtttgcacccaatttctggacattttatagctgagcattacatgttcttaaagggtcatgaaacttactcatattatgggcctctgaatttacttaccttcaatgtgggttatcataatgaacatcatgatttccccaacattcctggaaaaagtcttccactggtgaggaaaatagcagctgaatactatgacaacctccctcactacaattcctggataaaagtactgtatgattttgtgatggatgatacaataagtccctactcaagaatgaagaggcaccaaaaaggagagatggtgctggagtaaatatcattagtgccaaagggattcttctccaaaactttagatgataaaatggaatttttgcattattaaacttgagaccagtgatgctcagaagctcccctggcacaatttcagagtaagagctcggtgataccaagaagtgaatctggcttttaaacagtcagcctgactctgtactgctcagtttcactcacaggaaacttgtgacttgtgtattatcgtcattgaggatgtttcactcatgtctgtcattttataagcatatcatttaaaaagcttctaaaaagctatttcgccaggcacggtggctcatgcctataatcccagcactttgggaggccaaggtgggtggatcacctgaggtcaggagttcgagaccagcctggccaacacggtgaaaccccatctctactaaaaatgcaaaaattagccgggcgtggcggcacatgcctgtaatcccagctacatgggaggctgaggtgggagaattgcttgaacccaggaggcggaggcagaggctgcagtgacccaagattgtgccactgcactccaccctgggcaacagagcaagaccccatctcaaaaataaataaatatatataaaaaataaaaagctatttctagtttatttcactataaagttttgctttattaaaaagctaataaacagctattaatcacagtgtattagtatttgttacatttttgtatttcactatctttatactatataatatggtaacttgggtaccgggggaactttaaaatttcatctcaaaaataatttttaaaaagcctgaggtatgatatagcataaaagattgagatgaaaatatatttccctgtaagctgaattactcatttaaaaattttaacttctatatgggacccgaattagacactgctgaatcctgtacagccttactcataaataaagtacttactgaatttccaccattcaaaaaaaaaaaaaaaaaaaaaaaaaaaaaaaaaaaaaaaa

SEQ ID NO: 58 - protein, 323 aa, Homo sapiens
MGSRVSREDFEWVYTDQPHADRRREILAKYPEIKSLMKPDPNLIWIIIMMVLTQLGAFYIVKDLDWKWVIFGAYAFGSCINHSMTLAIHEIAHNAAFGNCKAMWNRWFGMFANLPIGIPYSISFKRYHMDHHRYLGADGVDVDIPTDFEGWFFCTAFRKFIWVILQPLFYAFRPLFINPKPITYLEVINTVAQVTFDILIYYFLGIKSLVYMLAASLLGLGLHPISGHFIAEHYMFLKGHETYSYYGPLNLLTFNVGYHNEHHDFPNIPGKSLPLVRKIAAEYYDNLPHYNSWIKVLYDFVMDDTISPYSRMKRHQKGEMVLE

SEQ ID NO: 59 - DNA, 13 bp, Artificial Sequence
  H-AP31 primer
aagcttggtgaac

SEQ ID NO: 60 - DNA, 16 bp, Artificial Sequence
  anchored oligo-dT primer
aagctttttttttttc

(19) United States
(12) Patent Application Publication
(10) Pub. No.: US 2008/0293134 A1
(43) Pub. Date: Nov. 27, 2008

(54) Human Cancer Suppressor Gene, Protein Encoded Therein, Expression Vector Containing Same

(76) Inventors: Hyun-Kee Kim, Seoul (KR); Jin-Woo Kim, Seoul (KR)

Correspondence Address: HAMRE, SCHUMANN, MUELLER & LARSON, P.C., P.O. BOX 2902, MINNEAPOLIS, MN, 55402-0902, US

(21) Appl. No.: 11/794,430

(22) PCT Filed: Dec. 28, 2005
(86) PCT No.: PCT/KR05/04618

§ 371 Date: Nov. 13, 2007

(30) Foreign Application Priority Data
Dec. 28, 2004 (KR) 10-2004-0114296

(51) Int. Cl. C12N 15/00; C07K 14/00; C12N 15/11

(52) U.S. Cl. 435/320.1; 530/350; 536/23.5

(57) ABSTRACT

Disclosed are a human cancer suppressor gene, a proteins encoded therein, an expression vectors containing the same, and a microorganism transformed with the vector. The genes of the present invention may be useful to diagnose and prevent the human cancers.

### TECHNICAL FIELD

The present invention relates to a human cancer suppressor gene, a protein encoded therein and an expression vector containing same.

### BACKGROUND ART

Tumor suppressor gene products function to suppress normal cells from being transformed into certain cancer cells, and therefore loss of this function of the tumor suppressor gene products allows the normal cells to become malignant transformants (Klein, G., FASEB J., 7, 821-825 (1993)). In order to allow cancer cells to grow into a cancer, the cells should lose a function to control the normal copy number of a tumor suppressor gene. It was found that modification in a coding sequence of a p53 tumor suppressor gene is one of the most general genetic changes in the human cancers (Bishop, J. M., Cell, 64, 235-248 (1991); and Weinberg, R. A., Science, 254, 1138-1146 (1991)). However, it was estimated that only some of the cervical cancer tissues exhibited a p53 mutation because the reported p53 mutation was only in a range of 2 to 11% in the cervical cancer (Crook, T. et al., Lancet, 339, 1070-1073 (1992); and Busby-Earle, R. M. C. et al., Br. j. Cancer, 69, 732-737 (1994)).

Meanwhile, it was reported that the mutation frequency of a p53 tumor suppressor gene in the non-small-cell lung cancer and the small-cell lung cancer amounted to approximately 50% and 70% of the lungs cancer, respectively (Takahashi, T. et al., Science, 246, 491-494 1989; Bodner, S. M. et al., Oncogene, 7, 743-749 (1992); Mao, L. Lung Cancer, 34, S27-S34 (2001)). Smoking is one of the most critical factors in development and progress of lung cancer, and other tumor suppressor genes and cancer genes are associated with these mutations together with the p53 (Osada, H. & Takahashi, T. Oncogene, 21, 7421-7434 (2002)).

Also, it was estimated that only some of breast cancer tissues exhibited a p53 mutation because the reported p53 mutation was in a range of 30% in the breast cancer (Keen, J. C. & Davidson, N. E., Cancer, 97, 825-833 (2003)) and Borresen-Dale, A-L., Human Mutation, 21, 292-300 (2003)).

Accordingly, the present inventors have ardently attempted to separate a novel tumor suppressor gene from normal tissues such as lungs, cervix and breast using an mRNA differential display (DD) method for effectively displaying genes differentially expressed between the normal tissues such as lungs, cervix and breast and the cancer tissues such as lung cancer, cervical cancer and breast cancer (Liang, P. and Pardee, A. B., Science, 257, 967-971 (1992); and Liang, P. et al., Cancer Res., 52, 6966-6968 (1993)).

### DISCLOSURE OF INVENTION

Accordingly, the present invention is designed to solve the problems of the prior art, and therefore it is an object of the present invention to provide a novel human cancer suppressor gene.

It is another object of the present invention to provide a cancer suppressor protein encoded by the cancer suppressor gene.

It is still another object of the present invention to provide an expression vector containing the cancer suppressor gene.

It is yet another object of the present invention to provide a cell transformed with the expression vector.

In order to accomplish the above object, the present invention provides a human cancer suppressor gene (so-called a growth-inhibiting gene 1; also referred to as GIG1) having a DNA sequence of SEQ ID NO: 1. The present invention provides a human cancer suppressor protein having an amino acid sequence of SEQ ID NO: 2.

Also, the present invention provides a human cancer suppressor gene (so-called a growth-inhibiting gene 3; also referred to as GIG3) having a DNA sequence of SEQ ID NO: 5. The present invention provides a human cancer suppressor protein having an amino acid sequence of SEQ ID NO: 6.

Also, the present invention provides a human cancer suppressor gene (so-called a growth-inhibiting gene 4; also referred to as GIG4) having a DNA sequence of SEQ ID NO: 9. The present invention provides a human cancer suppressor protein having an amino acid sequence of SEQ ID NO: 10.

Also, the present invention provides a human cancer suppressor gene (so-called a growth-inhibiting gene 5; also referred to as GIG5) having a DNA sequence of SEQ ID NO: 13. The present invention provides a human cancer suppressor protein having an amino acid sequence of SEQ ID NO: 14.

Also, the present invention provides a human cancer suppressor gene (so-called a growth-inhibiting gene 11; also referred to as GIG11) having a DNA sequence of SEQ ID NO: 17. The present invention provides a human cancer suppressor protein having an amino acid sequence of SEQ ID NO: 18.

Also, the present invention provides a human cancer suppressor gene (so-called a human migration-inducing gene 2; also referred to as MIG2) having a DNA sequence of SEQ ID NO: 21. The present invention provides a human cancer suppressor protein having an amino acid sequence of SEQ ID NO: 22.

Also, the present invention provides a human cancer suppressor gene (so-called a migration-inducing gene 4; also referred to as MIG4) having a DNA sequence of SEQ ID NO: 25. The present invention provides a human cancer suppressor protein having an amino acid sequence of SEQ ID NO: 26.

Also, the present invention provides a human cancer suppressor gene (so-called a proliferation-inducing gene 13; also referred to as PIG13) having a DNA sequence of SEQ ID NO: 29. The present invention provides a human cancer suppressor protein having an amino acid sequence of SEQ ID NO: 30.

Also, the present invention provides a human cancer suppressor gene (so-called a proliferation-inducing gene 15; also referred to as PIG15) having a DNA sequence of SEQ ID NO: 33. The present invention provides a human cancer suppressor protein having an amino acid sequence of SEQ ID NO: 34.

Also, the present invention provides a human cancer suppressor gene (so-called a proliferation-inducing gene 8; also referred to as PIG8) having a DNA sequence of SEQ ID NO: 37. The present invention provides a human cancer suppressor protein having an amino acid sequence of SEQ ID NO: 38.

Also, the present invention provides a human cancer suppressor gene (so-called a migration-related gene 1; also referred to as MRG1) having a DNA sequence of SEQ ID NO: 41. The present invention provides a human cancer suppressor protein having an amino acid sequence of SEQ ID NO: 42.

Also, the present invention provides a human cancer suppressor gene (so-called a proliferation-inducing gene 22; also referred to as PIG22) having a DNA sequence of SEQ ID NO: 45. The present invention provides a human cancer suppressor protein having an amino acid sequence of SEQ ID NO: 46.

Also, the present invention provides a human cancer suppressor gene (so-called a migration-inducing gene 9; also referred to as MIG9) having a DNA sequence of SEQ ID NO: 49. The present invention provides a human cancer suppressor protein having an amino acid sequence of SEQ ID NO: 50.

Also, the present invention provides a human cancer suppressor gene (so-called a migration-inducing gene 11; also referred to as MIG11) having a DNA sequence of SEQ ID NO: 53. The present invention provides a human cancer suppressor protein having an amino acid sequence of SEQ ID NO: 54.

Also, the present invention provides a human cancer suppressor gene (so-called a migration-inducing gene 15; also referred to as MIG15) having a DNA sequence of SEQ ID NO: 57. The present invention provides a human cancer suppressor protein having an amino acid sequence of SEQ ID NO: 58.

In order to accomplish the other object, the present invention provides an expression vector containing each of the cancer suppressor genes.

### BRIEF DESCRIPTION OF THE DRAWINGS

These and other features, aspects, and advantages of preferred embodiments of the present invention will be more fully described in the following detailed description, taken accompanying drawings. In the drawings:

FIG. 1 is a gel diagram showing a PCR result using a random 5′-13-mer primer H-AP38 of SEQ ID NO: 3 and an anchored oligo-dT primer of SEQ ID NO: 4;

FIG. 2 is a gel diagram showing a PCR result using a random 5′-13-mer primer H-AP32 of SEQ ID NO: 7 and an anchored oligo-dT primer of SEQ ID NO: 8;

FIG. 3 is a gel diagram showing a PCR result using a random 5′-13-mer primer H-AP35 of SEQ ID NO: 1 and an anchored oligo-dT primer of SEQ ID NO: 12;

FIG. 4 is a gel diagram showing a PCR result using a random 5′-13-mer primer H-AP34 of SEQ ID NO: 15 and an anchored oligo-dT primer of SEQ ID NO: 16;

FIG. 5 is a gel diagram showing a PCR result using a random 5′-13-mer primer H-AP36 of SEQ ID NO: 19 and an anchored oligo-dT primer of SEQ ID NO: 20;

FIG. 6 is a gel diagram showing a PCR result using a random 5′-13-mer primer H-AP35 of SEQ ID NO: 23 and an anchored oligo-dT primer of SEQ ID NO: 24;

FIG. 7 is a gel diagram showing a PCR result using a random 5′-13-mer primer H-AP41 of SEQ ID NO: 27 and an anchored oligo-dT primer of SEQ ID NO: 28;

FIG. 8 is a gel diagram showing a PCR result using a random 5′-13-mer primer H-AP6 of SEQ ID NO: 31 and an anchored oligo-dT primer of SEQ ID NO: 32;

FIG. 9 is a gel diagram showing a PCR result using a random 5′-13-mer primer H-AP6 of SEQ ID NO: 35 and an anchored oligo-dT primer of SEQ ID NO: 36;

FIG. 10 is a gel diagram showing a PCR result using a random 5′-13-mer primer H-AP36 of SEQ ID NO: 39 and an anchored oligo-dT primer of SEQ ID NO: 40;

FIG. 11 is a gel diagram showing a PCR result using a random 5′-13-mer primer H-AP21 of SEQ ID NO: 43 and an anchored oligo-dT primer of SEQ ID NO: 44;

FIG. 12 is a gel diagram showing a PCR result using a random 5′-13-mer primer H-AP24 of SEQ ID NO: 47 and an anchored oligo-dT primer of SEQ ID NO: 48;

FIG. 13 is a gel diagram showing a PCR result using a random 5′-13-mer primer H-AP12 of SEQ ID NO: 51 and an anchored oligo-dT primer of SEQ ID NO: 52;

FIG. 14 is a gel diagram showing a PCR result using a random 5′-13-mer primer H-AP13 of SEQ ID NO: 55 and an anchored oligo-dT primer of SEQ ID NO: 56;

FIG. 15 is a gel diagram showing a PCR result using a random 5′-13-mer primer H-AP31 of SEQ ID NO: 59 and an anchored oligo-dT primer of SEQ ID NO: 60;

FIGS. 16 to 30 are diagrams showing results that gene products of GIG1, GIG3, GIG4, GIG5, GIG11, MIG2, MIG4, PIG13, PIG15, PIG8, MRG1, PIG22, MIG9, MIG11 and MIG15 are analyzed on SDS-PAGE, respectively;

FIG. 31(a) is a diagram showing a northern blotting result that the GIG1 gene is differentially expressed in a normal exocervical tissue, a primary uterine cancer tissue and an uterine cancer cell line, and FIG. 31(b) is a diagram showing a northern blotting result obtained by hybridizing the same blot with β-actin probe;

FIG. 32(a) is a diagram showing a northern blotting result that the GIG3 gene is differentially expressed in a normal lung tissue, a primary lung cancer tissue and a lung cancer cell line, and FIG. 32(b) is a diagram showing a northern blotting result obtained by hybridizing the same blot with β-actin probe;

FIG. 33(a) is a diagram showing a northern blotting result that the GIG4 gene is differentially expressed in a normal lung tissue, a primary lung cancer tissue and a lung cancer cell line, and FIG. 33(b) is a diagram showing a northern blotting result obtained by hybridizing the same blot with β-actin probe;

FIG. 34(a) is a diagram showing a northern blotting result that the GIG5 gene is differentially expressed in a normal lung tissue, a primary lung cancer tissue and a lung cancer cell line, and FIG. 34(b) is a diagram showing a northern blotting result obtained by hybridizing the same blot with β-actin probe;

FIG. 35(a) is a diagram showing a northern blotting result that the GIG11 gene is differentially expressed in a normal breast tissue, a primary breast cancer tissue and a breast cancer cell line, and FIG. 35(b) is a diagram showing a northern blotting result obtained by hybridizing the same blot with β-actin probe;

FIG. 36(a) is a diagram showing a northern blotting result that the MIG2 gene is differentially expressed in a normal exocervical tissue, a primary uterine cancer tissue and an uterine cancer cell line, and FIG. 36(b) is a diagram showing a northern blotting result obtained by hybridizing the same blot with β-actin probe;

FIG. 37(a) is a diagram showing a northern blotting result that the MIG4 gene is differentially expressed in a normal exocervical tissue, a primary uterine cancer tissue and an uterine cancer cell line, and FIG. 37(b) is a diagram showing a northern blotting result obtained by hybridizing the same blot with β-actin probe;

FIG. 38(a) is a diagram showing a northern blotting result that the PIG13 gene is differentially expressed in a normal lung tissue, a primary lung cancer tissue and a lung cancer cell line, and FIG. 38(b) is a diagram showing a northern blotting result obtained by hybridizing the same blot with β-actin probe;

FIG. 39(a) is a diagram showing a northern blotting result that the PIG15 gene is differentially expressed in a normal lung tissue, a primary lung cancer tissue and a lung cancer cell line, and FIG. 39(b) is a diagram showing a northern blotting result obtained by hybridizing the same blot with β-actin probe;

FIG. 40(a) is a diagram showing a northern blotting result that the PIG8 gene is differentially expressed in a normal exocervical tissue, a primary uterine cancer tissue and an uterine cancer cell line, and FIG. 40(b) is a diagram showing a northern blotting result obtained by hybridizing the same blot with β-actin probe;

FIG. 41(a) is a diagram showing a northern blotting result that the MRG1 gene is differentially expressed in a normal exocervical tissue, a primary uterine cancer tissue and an uterine cancer cell line, and FIG. 41(b) is a diagram showing a northern blotting result obtained by hybridizing the same blot with β-actin probe;

FIG. 42(a) is a diagram showing a northern blotting result that the PIG22 gene is differentially expressed in a normal lung tissue, a primary lung cancer tissue and a lung cancer cell line, and FIG. 42(b) is a diagram showing a northern blotting result obtained by hybridizing the same blot with β-actin probe;

FIG. 43(a) is a diagram showing a northern blotting result that the MIG9 gene is differentially expressed in a normal lung tissue, a primary lung cancer tissue and a lung cancer cell line, and FIG. 43(b) is a diagram showing a northern blotting result obtained by hybridizing the same blot with β-actin probe;

FIG. 44(a) is a diagram showing a northern blotting result that the MIG11 gene is differentially expressed in a normal lung tissue, a primary lung cancer tissue and a lung cancer cell line, and FIG. 44(b) is a diagram showing a northern blotting result obtained by hybridizing the same blot with β-actin probe;

FIG. 45(a) is a diagram showing a northern blotting result that the MIG15 gene is differentially expressed in a normal exocervical tissue, a primary uterine cancer tissue and an uterine cancer cell line, and FIG. 45(b) is a diagram showing a northern blotting result obtained by hybridizing the same blot with β-actin probe;

FIG. 46(a) is a diagram showing a northern blotting result that the GIG1 gene is differentially expressed in various normal tissues, and FIG. 46(b) is a diagram showing a northern blotting result obtained by hybridizing the same blot with β-actin probe;

FIG. 47(a) is a diagram showing a northern blotting result that the GIG3 gene is differentially expressed in various normal tissues, and FIG. 47(b) is a diagram showing a northern blotting result obtained by hybridizing the same blot with β-actin probe;

FIG. 48(a) is a diagram showing a northern blotting result that the GIG4 gene is differentially expressed in various normal tissues, and FIG. 48(b) is a diagram showing a northern blotting result obtained by hybridizing the same blot with β-actin probe;

FIG. 49(a) is a diagram showing a northern blotting result that the GIG5 gene is differentially expressed in various normal tissues, and FIG. 49(b) is a diagram showing a northern blotting result obtained by hybridizing the same blot with β-actin probe;

FIG. 50(a) is a diagram showing a northern blotting result that the GIG11 gene is differentially expressed in various normal tissues, and FIG. 50(b) is a diagram showing a northern blotting result obtained by hybridizing the same blot with β-actin probe;

FIG. 51(a) is a diagram showing a northern blotting result that the MIG2 gene is differentially expressed in various normal tissues, and FIG. 51(b) is a diagram showing a northern blotting result obtained by hybridizing the same blot with β-actin probe;

FIG. 52(a) is a diagram showing a northern blotting result that the MIG4 gene is differentially expressed in various normal tissues, and FIG. 52(b) is a diagram showing a northern blotting result obtained by hybridizing the same blot with β-actin probe;

FIG. 53(a) is a diagram showing a northern blotting result that the PIG13 gene is differentially expressed in various normal tissues, and FIG. 53(b) is a diagram showing a northern blotting result obtained by hybridizing the same blot with β-actin probe;

FIG. 54(a) is a diagram showing a northern blotting result that the PIG15 gene is differentially expressed in various normal tissues, and FIG. 54(b) is a diagram showing a northern blotting result obtained by hybridizing the same blot with β-actin probe;

FIG. 55(a) is a diagram showing a northern blotting result that the PIG8 gene is differentially expressed in various normal tissues, and FIG. 55(b) is a diagram showing a northern blotting result obtained by hybridizing the same blot with β-actin probe;

FIG. 56(a) is a diagram showing a northern blotting result that the MRG1 gene is differentially expressed in various normal tissues, and FIG. 56(b) is a diagram showing a northern blotting result obtained by hybridizing the same blot with β-actin probe;

FIG. 57(a) is a diagram showing a northern blotting result that the PIG22 gene is differentially expressed in various normal tissues, and FIG. 57(b) is a diagram showing a northern blotting result obtained by hybridizing the same blot with β-actin probe;

FIG. 58(a) is a diagram showing a northern blotting result that the MIG9 gene is differentially expressed in various normal tissues, and FIG. 58(b) is a diagram showing a northern blotting result obtained by hybridizing the same blot with β-actin probe;

FIG. 59(a) is a diagram showing a northern blotting result that the MIG11 gene is differentially expressed in various normal tissues, and FIG. 59(b) is a diagram showing a northern blotting result obtained by hybridizing the same blot with β-actin probe;

FIG. 60(a) is a diagram showing a northern blotting result that the MIG15 gene is differentially expressed in various normal tissues, and FIG. 60(b) is a diagram showing a northern blotting result obtained by hybridizing the same blot with β-actin probe;

FIG. 61(a) is a diagram showing a northern blotting result that the GIG1 gene is differentially expressed in various cancer cell lines, and FIG. 61(b) is a diagram showing a northern blotting result obtained by hybridizing the same blot with β-actin probe;

FIG. 62(a) is a diagram showing a northern blotting result that the GIG3 gene is differentially expressed in various cancer cell lines, and FIG. 62(b) is a diagram showing a northern blotting result obtained by hybridizing the same blot with β-actin probe;

FIG. 63(a) is a diagram showing a northern blotting result that the GIG4 gene is differentially expressed in various cancer cell lines, and FIG. 63(b) is a diagram showing a northern blotting result obtained by hybridizing the same blot with β-actin probe;

FIG. 64(a) is a diagram showing a northern blotting result that the GIG5 gene is differentially expressed in various cancer cell lines, and FIG. 64(b) is a diagram showing a northern blotting result obtained by hybridizing the same blot with β-actin probe;

FIG. 65(a) is a diagram showing a northern blotting result that the GIG11 gene is differentially expressed in various cancer cell lines, and FIG. 65(b) is a diagram showing a northern blotting result obtained by hybridizing the same blot with β-actin probe;

FIG. 66(a) is a diagram showing a northern blotting result that the MIG2 gene is differentially expressed in various cancer cell lines, and FIG. 66(b) is a diagram showing a northern blotting result obtained by hybridizing the same blot with β-actin probe;

FIG. 67(a) is a diagram showing a northern blotting result that the MIG4 gene is differentially expressed in various cancer cell lines, and FIG. 67(b) is a diagram showing a northern blotting result obtained by hybridizing the same blot with β-actin probe;

FIG. 68(a) is a diagram showing a northern blotting result that the PIG13 gene is differentially expressed in various cancer cell lines, and FIG. 68(b) is a diagram showing a northern blotting result obtained by hybridizing the same blot with β-actin probe;

FIG. 69(a) is a diagram showing a northern blotting result that the PIG15 gene is differentially expressed in various cancer cell lines, and FIG. 69(b) is a diagram showing a northern blotting result obtained by hybridizing the same blot with β-actin probe;

FIG. 70(a) is a diagram showing a northern blotting result that the PIG8 gene is differentially expressed in various cancer cell lines, and FIG. 70(b) is a diagram showing a northern blotting result obtained by hybridizing the same blot with β-actin probe;

FIG. 71(a) is a diagram showing a northern blotting result that the MRG1 gene is differentially expressed in various cancer cell lines, and FIG. 71(b) is a diagram showing a northern blotting result obtained by hybridizing the same blot with β-actin probe;

FIG. 72(a) is a diagram showing a northern blotting result that the PIG22 gene is differentially expressed in various cancer cell lines, and FIG. 72(b) is a diagram showing a northern blotting result obtained by hybridizing the same blot with β-actin probe;

FIG. 73(a) is a diagram showing a northern blotting result that the MIG9 gene is differentially expressed in various cancer cell lines, and FIG. 73(b) is a diagram showing a northern blotting result obtained by hybridizing the same blot with β-actin probe;

FIG. 74(a) is a diagram showing a northern blotting result that the MIG11 gene is differentially expressed in various cancer cell lines, and FIG. 74(b) is a diagram showing a northern blotting result obtained by hybridizing the same blot with β-actin probe;

FIG. 75(a) is a diagram showing a northern blotting result that the MIG15 gene is differentially expressed in various cancer cell lines, and FIG. 75(b) is a diagram showing a northern blotting result obtained by hybridizing the same blot with β-actin probe;

FIG. 76 is a diagram showing growth curves of a wild-type HeLa cell, a HeLa uterine cancer cell transfected with the GIG1 gene, and a HeLa cell transfected with the expression vector pcDNA3.1;

FIG. 77 is a diagram showing growth curves of a wild-type A549 lung cancer cell line, an A549 lung cancer cell transfected with the GIG3 gene, and an A549 cell transfected with the expression vector pcDNA3.1;

FIG. 78 is a diagram showing growth curves of a wild-type A549 lung cancer cell line, an A549 lung cancer cell transfected with the GIG4 gene, and an A549 cell transfected with the expression vector pcDNA3.1;

FIG. 79 is a diagram showing growth curves of an A549 lung cancer cell line, an A549 lung cancer cell transfected with the GIG5 gene, and an A549 cell transfected with the expression vector pcDNA3.1;

FIG. 80 is a diagram showing growth curves of a wild-type MCF-7 cell, an MCF-7 breast cancer cell transfected with the GIG11 gene, and an MCF-7 cell transfected with the expression vector pcDNA3.1;

FIG. 81 is a diagram showing growth curves of a wild-type HeLa cell, a HeLa uterine cancer cell transfected with the MIG2 gene, and a HeLa cell transfected with the expression vector pcDNA3.1;

FIG. 82 is a diagram showing growth curves of a wild-type HeLa cell, a HeLa uterine cancer cell transfected with the MIG4 gene, and a HeLa cell transfected with the expression vector pcDNA3.1;

FIG. 83 is a diagram showing growth curves of a wild-type A549 lung cancer cell line, an A549 lung cancer cell transfected with the PIG13 gene, and an A549 cell transfected with the expression vector pcDNA3.1;

FIG. 84 is a diagram showing growth curves of a wild-type A549 lung cancer cell line, an A549 lung cancer cell transfected with the PIG15 gene, and an A549 cell transfected with the expression vector pcDNA3.1;

FIG. 85 is a diagram showing growth curves of a wild-type HeLa cell, a HeLa uterine cancer cell transfected with the PIG8 gene, and a HeLa cell transfected with the expression vector pcDNA3.1;

FIG. 86 is a diagram showing growth curves of a wild-type HeLa cell, a HeLa uterine cancer cell transfected with the MRG1 gene, and a HeLa cell transfected with the expression vector pcDNA3.1;

FIG. 87 is a diagram showing growth curves of a wild-type A549 lung cancer cell line, an A549 lung cancer cell transfected with the PIG22 gene, and an A549 cell transfected with the expression vector pcDNA3.1;

FIG. 88 is a diagram showing growth curves of a wild-type A549 lung cancer cell line, an A549 lung cancer cell transfected with the MIG9 gene, and an A549 cell transfected with the expression vector pcDNA3.1;

FIG. 89 is a diagram showing growth curves of a wild-type A549 lung cancer cell line, an A549 lung cancer cell transfected with the MIG11 gene, and an A549 cell transfected with the expression vector pcDNA3.1; and

FIG. 90 is a diagram showing growth curves of a wild-type HeLa cell, a HeLa uterine cancer cell transfected with the MIG15 gene, and a HeLa cell transfected with the expression vector pcDNA3.1.

### BEST MODES FOR CARRYING OUT THE INVENTION

Hereinafter, the present invention will be described in detail with reference to the accompanying drawings.

1. GIG1

The gene of the present invention is a human cancer suppressor gene 1 (GIG1) having a DNA sequence of SEQ ID NO: 1, which was deposited with Accession No. AY268890 into the GenBank database of U.S. National Institutes of Health (NIH) (Publication Date: Dec. 31, 2004), and some DNA sequence of the deposited gene is identical with that of the Homo sapiens ceroid-lipofuscinosis, neuronal 2, late infantile (Jansky-Bielschowsky disease) (CLN2) deposited with Accession No. NM_{—}000391 into the database.

The DNA sequence of SEQ ID NO: 1 has one open reading frame (ORF) corresponding to base positions from 800 to 1762 of the DNA sequence (base positions from 1760 to 1762 represent a stop codon).

The protein expressed from the gene of the present invention consists of 320 amino acid residues, and has an amino acid sequence of SEQ ID NO: 2 and a molecular weight of approximately 34 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 1. As another example, a 382-bp cDNA fragment (corresponding to base positions from 3109 to 3490), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP38 of SEQ ID NO: 3 (5′-AAGCTTCCAGTGC-3′) and an anchored oligo-dT primer of SEQ ID NO: 4 (5′-AAGCTTTTTTTTTTTC-3′), and the resultant fragment, which is used as the probe, may be plaque-hybridized with a cDNA library to obtain a full-length cDNA clone.

It is regarded that the gene of the present invention is overexpressed in the normal tissues, preferably uterus, brain, skeletal muscles, spleen, kidney, liver, placenta, lungs, and peripheral blood leukocyte, to suppress carcinogenesis. The gene of the present invention is mainly overexpressed in these tissues as an mRNA transcript having a size of approximately 4.0 kb, and an transcript having a size of approximately 3.0 kb is also expressed in addition to the 4.0-kb mRNA transcript. Especially, the gene of the present invention is differentially expressed only in the normal tissues. For example, the gene of the present invention is slightly expressed or not detected in the cancer tissues and the cancer cells such as the uterine cancer tissue and the uterine cancer cell line, but differentially expressed only in the normal tissues.

The uterine cancer cell line into which the genes of the present invention are introduced showed a high mortality, and therefore the gene of the present invention may be effectively used for treatment and prevention of the cancer.

2. GIG3

The gene of the present invention is a human cancer suppressor gene 3 (GIG3) having a DNA sequence of SEQ ID NO: 5, which was deposited with Accession No. AY423721 into the GenBank database of U.S. National Institutes of Health (NIH) (Publication Date: Dec. 31, 2004), and some DNA sequence of the deposited gene is identical with that of the Homo sapiens Fas (TNFRSF6)-associated via death domain (FADD) deposited with Accession No. NM_{—}003824 into the database.

The DNA sequence of SEQ ID NO: 5 has one open reading frame (ORF) corresponding to base positions from 1 to 627 of the DNA sequence (base positions from 625 to 627 represent a stop codon).

The protein expressed from the gene of the present invention consists of 208 amino acid residues, and has an amino acid sequence of SEQ ID NO: 6 and a molecular weight of approximately 23 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 5. As another example, a 190-bp cDNA fragment, which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP32 of SEQ ID NO: 7 (5′-AAGCTTCTTGCAA-3′) and an anchored oligo-dT primer of SEQ ID NO: 8 (5′-AAGCTTTTTTTTTTTC-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, kidney, and liver, to suppress carcinogenesis. The gene of the present invention is mainly overexpressed in these tissues as an mRNA transcript having a size of approximately 1.3 kb, and an transcript having a size of approximately 0.7 kb is also expressed in addition to the 4.0-kb mRNA transcript. Especially, the gene of the present invention is differentially expressed only in the normal tissues. For example, the gene of the present invention is slightly expressed or not detected in the cancer tissues and the cancer cells such as the lung cancer tissue, the metastatic lung cancer tissue and the lung cancer cell lines (A549 and NCI-H358), but differentially increasingly expressed only in the normal lung tissues. The cancer cell line into which the genes of the present invention are introduced showed a high mortality, and therefore the gene of the present invention may be effectively used for treatment and prevention of the cancer.

3. GIG4

The gene of the present invention is a human cancer suppressor gene 4 (GIG4) having a DNA sequence of SEQ ID NO: 9, which was deposited with Accession No. AY423722 into the GenBank database of U.S. National Institutes of Health (NIH) (Publication Date: Dec. 31, 2004), and the DNA sequence of the deposited gene is identical with that of the Homo sapiens RAB13, member RAS oncogene family (RAB13) deposited with Accession No. NM_{—}002870 into the database.

Contrary to the functions of the RAB13 as reported previously, it was however found from this study result that a GIG4 tumor suppressor gene was slightly expressed in various human tumors including the lung cancer, while its expression was significantly increased in various normal tissues.

The DNA sequence of SEQ ID NO: 9 has one open reading frame (ORF) corresponding to base positions from 2 to 613 of the DNA sequence (base positions from 611 to 613 represent a stop codon).

The protein expressed from the gene of the present invention consists of 203 amino acid residues, and has an amino acid sequence of SEQ ID NO: 10 and a molecular weight of approximately 23 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 9. As another example, a 187-bp cDNA fragment, which is very slightly expressed in the cancer tissue or the cancer cell line but differentially increasingly expressed in the normal tissue, may be obtained by carrying out a reverse transcription-polymerase chain reaction (RT-PCR) on the total RNA extracted from a normal tissue, and a cancer tissue or a cancer cell line using a random primer H-AP35 of SEQ ID NO: 11 (5′-AAGCTTCAGGGCA-3′) and an anchored oligo-dT primer of SEQ ID NO: 12 (5′-AAGCTTTTTTTTTTTC-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, kidney, and liver, to suppress carcinogenesis. The gene of the present invention is mainly overexpressed in these tissues as an mRNA transcript having a size of approximately 1.3 kb. Especially, the gene of the present invention is differentially expressed only in the normal tissues. For example, the gene of the present invention is slightly expressed in the cancer tissues and the cancer cells such as the lung cancer tissue, the metastatic lung cancer tissue and the lung cancer cell lines (A549 and NCI-H358), but differentially increasingly expressed only in the normal lung tissues. The cancer cell line into which the genes of the present invention are introduced showed a high mortality, and therefore the gene of the present invention may be effectively used for treatment and prevention of the cancer.

4. GIG 5

The gene of the present invention is a human cancer suppressor gene 3 (GIG3) having a DNA sequence of SEQ ID NO: 13, which was deposited with Accession No. AY423723 into the GenBank database of U.S. National Institutes of Health (NIH) (Publication Date: Dec. 31, 2004), and the DNA sequence of the deposited gene is identical with that of the Homo sapiens calcyclin binding protein (CACYBP), transcript variant 1 deposited with Accession No. ACCESSION NM_{—}014412 into the database.

Contrary to the functions of the SIP as reported previously, it was however found from this study result that a GIG5 tumor suppressor gene was slightly expressed in various human tumors including the lung cancer, while its expression was significantly increased in various normal tissues.

The DNA sequence of SEQ ID NO: 13 has one open reading frame (ORF) corresponding to base positions from 2 to 688 of the DNA sequence (base positions from 686 to 688 represent a stop codon).

The protein expressed from the gene of the present invention consists of 228 amino acid residues, and has an amino acid sequence of SEQ ID NO: 14 and a molecular weight of approximately 26 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 13. As another example, a 212-bp cDNA fragment, which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP34 of SEQ ID NO: 15 (5′-AAGCTTCAGCAGC-3′) and an anchored oligo-dT primer of SEQ ID NO: 16 (5′-AAGCTTTTTTTTTTTC-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, kidney, and liver, to suppress carcinogenesis. The gene of the present invention is mainly overexpressed in these tissues as an mRNA transcript having a size of approximately 1.2 kb, and an transcript having a size of approximately 2.0 kb is also expressed in addition to the 1.2-kb mRNA transcript. Especially, the gene of the present invention is differentially expressed only in the normal tissues. For example, the gene of the present invention is slightly expressed in the cancer tissues and the cancer cells such as the lung cancer tissue, the metastatic lung cancer tissue and the lung cancer cell lines (A549 and NCI-H358), but differentially increasingly expressed only in the normal lung tissues. The cancer cell line into which the genes of the present invention are introduced showed a high mortality, and therefore the gene of the present invention may be effectively used for treatment and prevention of the cancer.

5. GIG 11

The gene of the present invention is a human cancer suppressor gene 11 (GIG11) having a DNA sequence of SEQ ID NO: 17, which was deposited with Accession No. AY451236 into the GenBank database of U.S. National Institutes of Health (NIH) (Publication Date: Dec. 31, 2004), and some DNA sequence of the deposited gene is identical with those of the full-length cDNA clone CS0DD008YG11 of Neuroblastoma Cot 50-normalized of Homo sapiens (human) gene, the Homo sapiens thioredoxin-related transmembrane protein 2 gene and the Homo sapiens CGI-31 protein mRNA gene, all deposited with Accession No. CR614679, BC000666 and AF132965 into the database, respectively.

The DNA sequence of SEQ ID NO: 17 has one open reading frame (ORF) corresponding to base positions from 16 to 768 of the DNA sequence (base positions from 766 to 768 represent a stop codon).

The protein expressed from the gene of the present invention consists of 250 amino acid residues, and has an amino acid sequence of SEQ ID NO: 18 and a molecular weight of approximately 29 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 17. As another example, a 298-bp cDNA fragment, which is not expressed in the cancer tissue or the cancer cell line but differentially increasingly expressed in the normal tissue, may be obtained by carrying out a reverse transcription-polymerase chain reaction (RT-PCR) on the total RNA extracted from a normal tissue, and a cancer tissue or a cancer cell line using a random primer H-AP36 of SEQ ID NO: 19 (5′-AAGCTTCGACGCT-3′) and an anchored oligo-dT primer of SEQ ID NO: 20 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably breast, brain, heart, muscles, thymus, spleen, kidney, liver, small intestines, placenta and lungs, to suppress carcinogenesis. The gene of the present invention is mainly overexpressed in these tissues as an mRNA transcript having a size of approximately 1.5 kb. Especially, the gene of the present invention is differentially expressed only in the normal tissues. For example, the gene of the present invention is slightly expressed in the cancer tissues and the cancer cells such as the breast cancer tissue, the breast cancer cell line MCF-7, etc., but differentially increasingly expressed only in the normal breast tissues.

The cancer cell line into which the genes of the present invention are introduced showed a high mortality, and therefore the gene of the present invention may be effectively used for treatment and prevention of the cancer.

6. MIG2

A DNA sequence of SEQ ID NO: 21 was deposited with Accession No. AY237654 into the GenBank database of U.S. National Institutes of Health (NIH) (Publication Date: Dec. 31, 2004), and the DNA sequencing analysis showed that the DNA sequence of the deposited gene is similar to those of the Homo sapiens KIAA0084 mRNA and Homo sapiens raft-linking protein (RAFTLIN), both deposited with Accession No. D42043 and NM_{—}015150 XM_{—}042841 into the database and its expressed amino acid sequence is also similar to those of the Homo sapiens KIAA0084 mRNA and Homo sapiens raft-linking protein (RAFTLIN).

The DNA sequence of SEQ ID NO: 21 has one open reading frame (ORF) corresponding to base positions from 274 to 2010 of the DNA sequence (base positions from 2008 to 2010 represent a stop codon).

The protein expressed from the gene of the present invention consists of 578 amino acid residues, and has an amino acid sequence of SEQ ID NO: 22 and a molecular weight of approximately 63 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 21. As another example, a 311-bp cDNA fragment (corresponding to base positions from 2671 to 2981), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP35 of SEQ ID NO: 23 (5′-AAGCTTCAGGGCA-3′) and an anchored oligo-dT primer of SEQ ID NO: 24 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably uterus, heart, skeletal muscles, kidney and liver, to suppress carcinogenesis. The gene of the present invention is mainly overexpressed in these tissues as an mRNA transcript having a size of approximately 5.0 kb, and an transcript having a size of approximately 2.0 kb is also expressed in addition to the 5.0-kb mRNA transcript. Especially, the gene of the present invention is differentially expressed only in the normal tissues. For example, the gene of the present invention is not expressed or slightly expressed in the cancer tissues and the cancer cells such as the uterine cancer tissue and the uterine cancer cell line, but differentially highly expressed only in the normal uterine tissues.

The cancer cell line into which the genes of the present invention are introduced showed a high mortality, and therefore the gene of the present invention may be effectively used for treatment and prevention of the cancer.

7. MIG 4

The gene of the present invention is a human cancer suppressor gene (MIG4) having a DNA sequence of SEQ ID NO: 25, which was deposited with Accession No. AY260745 into the GenBank database of U.S. National Institutes of Health (NIH) (Publication Date: Dec. 31, 2004), and it was revealed that the DNA sequence of the deposited gene is similar to that of the Homo sapiens aminolevulinate, delta-, synthase 1 (ALAS1), transcript variant 1 gene deposited with Accession No. NM_{—}000688 into the database.

The DNA sequence of SEQ ID NO: 25 has one open reading frame (ORF) corresponding to base positions from 322 to 2244 of the DNA sequence (base positions from 320 to 322 represent a stop codon).

The protein expressed from the gene of the present invention consists of 640 amino acid residues, and has an amino acid sequence of SEQ ID NO: 26 and a molecular weight of approximately 70 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 25. As another example, a 322-bp cDNA fragment (corresponding to base positions from 1908 to 2229), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP31 of SEQ ID NO: 27 (5′-AAGCTTGGTGAAC-3′) and an anchored oligo-dT primer of SEQ ID NO: 28 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably uterus, brain, heart, skeletal muscles, large intestines, spleen, kidney, liver, placenta, lungs and peripheral blood leukocyte, to suppress carcinogenesis. The gene of the present invention is mainly overexpressed in these tissues as an mRNA transcript having a size of approximately 0.5 kb, and an transcript having a size of approximately 2.0 kb is also expressed in addition to the 0.5-kb mRNA transcript. Especially, the gene of the present invention is differentially expressed only in the normal tissues. For example, the gene of the present invention is not expressed or slightly expressed in the cancer tissues and the cancer cells such as the uterine cancer tissue and the uterine cancer cell line, but differentially highly expressed only in the normal uterine tissues.

The cancer cell line into which the genes of the present invention are introduced showed a high mortality, and therefore the gene of the present invention may be effectively used for treatment and prevention of the cancer.

8. PIG13

The gene of the present invention is a human cancer suppressor gene (PIG13) having a DNA sequence of SEQ ID NO: 29, which was deposited with Accession No. AY258286 into the GenBank database of U.S. National Institutes of Health (NIH) (Publication Date: Dec. 31, 2004), and it was revealed that the DNA sequence of the deposited gene is similar to those of the Homo sapiens cDNA FLJ31925 fis, clone NT2RP7005493 gene, the Homo sapiens chromosome 1 open reading frame 21, mRNA (cDNA clone MGC:16172 IMAGE:3635521) gene and the Homo sapiens Clorf21 mRNA gene, all deposited with Accession No. AK056487, BC028567 and AF312864 into the database, respectively.

The DNA sequence of SEQ ID NO: 29 has one open reading frame (ORF) corresponding to base positions from 391 to 756 of the DNA sequence (base positions from 754 to 756 represent a stop codon).

The protein expressed from the gene of the present invention consists of 121 amino acid residues, and has an amino acid sequence of SEQ ID NO: 30 and a molecular weight of approximately 14 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 1. As another example, a 296-bp cDNA fragment, which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP6 of SEQ ID NO: 31 (5′-AAGCTTGCACCAT-3′) and an anchored oligo-dT primer of SEQ ID NO: 32 (5′-AAGCTTTTTTTTTTTG-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, spleen, kidney and liver, to suppress carcinogenesis. The gene of the present invention is mainly overexpressed in these tissues as an mRNA transcript having a size of approximately 1.0 kb, and an transcript having a size of approximately 4.5 kb is also expressed in addition to the 1.0-kb mRNA transcript. Especially, the gene of the present invention is differentially expressed in the normal tissues. For example, the gene of the present invention is slightly expressed in the cancer tissues and the cancer cells such as the lung cancer tissue, the metastatic lung cancer tissue, the lung cancer cell line (A549 and NCI-H358), etc., but differentially highly expressed only in the normal lung tissues. The cancer cell line into which the genes of the present invention are introduced showed a high mortality, and therefore the gene of the present invention may be effectively used for treatment and prevention of the cancer.

9. PIG15

The gene of the present invention is a human cancer suppressor gene (PIG15) having a DNA sequence of SEQ ID NO: 33, which was deposited with Accession No. AY258285 into the GenBank database of U.S. National Institutes of Health (NIH) (Publication Date: Dec. 31, 2004), and it was revealed that the DNA sequence of the deposited gene is similar to those of the Human ferritin heavy chain mRNA gene and the Human ferritin heavy chain mRNA gene, both deposited with Accession No. L20941 and M97164 into the database, respectively.

The DNA sequence of SEQ ID NO: 33 has one open reading frame (ORF) corresponding to base positions from 794 to 1345 of the DNA sequence (base positions from 1343 to 1345 represent a stop codon).

The protein expressed from the gene of the present invention consists of 183 amino acid residues, and has an amino acid sequence of SEQ ID NO: 34 and a molecular weight of approximately 21 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 33. As another example, a 327-bp cDNA fragment, which is not expressed in the cancer tissue or the cancer cell line but differentially expressed only in the normal tissue, may be obtained by carrying out a reverse transcription-polymerase chain reaction (RT-PCR) on the total RNA extracted from a normal tissue, and a cancer tissue or a cancer cell line using a random primer H-AP6 of SEQ ID NO: 35 (5′-AAGCTTGCACCAT-3′) and an anchored oligo-dT primer of SEQ ID NO: 36 (5′-AAGCTTTTTTTTTTTG-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, spleen, kidney and liver, to suppress carcinogenesis. The gene of the present invention is mainly overexpressed in these tissues as an mRNA transcript having a size of approximately 1.0 kb. Especially, the gene of the present invention is differentially expressed in the normal tissues. For example, the gene of the present invention is slightly expressed in the cancer tissues and the cancer cells such as the lung cancer tissue, the metastatic lung cancer tissue, the lung cancer cell line (A549 and NCI-H358), etc., but differentially highly expressed only in the normal lung tissues. The cancer cell line into which the genes of the present invention are introduced showed a high mortality, and therefore the gene of the present invention may be effectively used for treatment and prevention of the cancer.

10. PIG8

The gene of the present invention is a human cancer suppressor gene (PIG8) having a DNA sequence of SEQ ID NO: 37, which was deposited with Accession No. AY239292 into the GenBank database of U.S. National Institutes of Health (NIH) (Publication Date: Dec. 31, 2004), and it was revealed that some DNA sequence of the deposited gene is similar to those of the Homo sapiens KIAA0092 mRNA, the Homo sapiens genomic DNA, chromosome 11 clone:CTD-2564P9 and the Homo sapiens translokin (KIAA0092) gene, all deposited with Accession No. D42054, AP001877 and NM_{—}014679 XM_{—}374925 into the database, respectively.

The DNA sequence of SEQ ID NO: 37 has one open reading frame (ORF) corresponding to base positions from 140 to 1642 of the DNA sequence (base positions from 1640 to 1642 represent a stop codon).

The protein expressed from the gene of the present invention consists of 500 amino acid residues, and has an amino acid sequence of SEQ ID NO: 38 and a molecular weight of approximately 57 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 37. As another example, a 362-bp cDNA fragment (corresponding to base positions from 2586 to 2947), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP36 of SEQ ID NO: 39 (5′-AAGCTTGGTGAAC-3′) and an anchored oligo-dT primer of SEQ ID NO: 40 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably uterus, brain, heart, skeletal muscles, liver, placenta and peripheral blood leukocyte, to suppress carcinogenesis. The gene of the present invention is mainly overexpressed in these tissues as an mRNA transcript having a size of approximately 4.5 kb, and an transcript having a size of approximately 2.2 kb is also expressed additionally in the normal liver tissue. Especially, the gene of the present invention is differentially expressed only in the normal tissues. For example, the gene of the present invention is not expressed or slightly expressed in the cancer tissues and the cancer cells such as the uterine cancer tissue and the uterine cancer cell line, but differentially highly expressed only in the normal uterine tissues. The uterine cancer cell line into which the genes of the present invention are introduced showed a high mortality, and therefore the gene of the present invention may be effectively used for treatment and prevention of the cancer.

The gene of the present invention is a human cancer suppressor gene (MRG1) having a DNA sequence of SEQ ID NO: 41, which was deposited with Accession No. AY423731 into the GenBank database of U.S. National Institutes of Health (NIH) (Publication Date: Dec. 31, 2004), and some DNA sequence of the deposited gene is identical with those of the Homo sapiens membrane protein, palmitoylated 1, 55 kDa (MPP1) gene and the Homo sapiens membrane protein, palmitoylated 1, 55 kDa gene, both deposited with Accession No. NM_{—}002436 and BC002392 into the database, respectively.

The DNA sequence of SEQ ID NO: 41 has one open reading frame (ORF) corresponding to base positions from 27 to 1427 of the DNA sequence (base positions from 1425 to 1427 represent a stop codon).

The protein expressed from the gene of the present invention consists of 466 amino acid residues, and has an amino acid sequence of SEQ ID NO: 42 and a molecular weight of approximately 52 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 41. As another example, a 277-bp cDNA fragment (corresponding to base positions from 1123 to 1399), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP21 of SEQ ID NO: 43 (5′-AAGCTTTCTCTGG-3′) and an anchored oligo-dT primer of SEQ ID NO: 44 (5′-AAGCTTTTTTTTTTTG-3′), and the resultant fragment, which is used as the probe, may be plaque-hybridized with a cDNA library to obtain a full-length cDNA clone.

It is regarded that the gene of the present invention is overexpressed in the normal tissues, preferably uterus, brain, skeletal muscles, spleen, kidney, liver, placenta, lungs and peripheral blood leukocyte, to suppress carcinogenesis. The gene of the present invention is mainly overexpressed in these tissues as an mRNA transcript having a size of approximately 5.0 kb, and an transcript having a size of approximately 2.0 kb is also expressed in addition to the 5.0-kb mRNA transcript. Especially, the gene of the present invention is differentially expressed only in the normal tissues. For example, the gene of the present invention is not expressed or slightly expressed in the cancer tissues and the cancer cells such as the uterine cancer tissue and the uterine cancer cell line, but differentially highly expressed only in the normal uterine tissues. The uterine cancer cell line into which the genes of the present invention are introduced showed a high mortality, and therefore the gene of the present invention may be effectively used for treatment and prevention of the cancer.

12. PIG22

The gene of the present invention is a human cancer suppressor gene (PIG22) having a DNA sequence of SEQ ID NO: 45, which was deposited with Accession No. AY423729 into the GenBank database of U.S. National Institutes of Health (NIH) (Publication Date: Dec. 31, 2004), and it was revealed that the DNA sequence of the deposited gene is identical with those of the Homo sapiens cDNA clone IMAGE:5295100 gene, the Homo sapiens cDNA FLJ13851 fis, clone THYRO1000926, highly similar to Homo sapiens cAMP-specific phosphodiesterase 8B (PDE8B) gene, and the Homo sapiens HSPDE8B4 mRNA for phosphodiesterase 8B4 gene, all deposited with Accession No. BC043209, AK023913 and AB085827 into the database, respectively. However, it was however found from this study result that the PIG22 tumor suppressor gene was slightly expressed in various human tumors including the lung cancer, while its expression was significantly increased in various normal tissues.

The DNA sequence of SEQ ID NO: 45 has one open reading frame (ORF) corresponding to base positions from 11 to 1063 of the DNA sequence (base positions from 1061 to 1063 represent a stop codon).

The protein expressed from the gene of the present invention consists of 350 amino acid residues, and has an amino acid sequence of SEQ ID NO: 46 and a molecular weight of approximately 40 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 45. As another example, a 242-bp cDNA fragment, which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP24 of SEQ ID NO: 47 (5′-AAGCTTCACTAGC-3′) and an anchored oligo-dT primer of SEQ ID NO: 48 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, liver and placenta, to suppress carcinogenesis. The gene of the present invention is mainly overexpressed in these tissues as an mRNA transcript having a size of approximately 5 kb, and an transcript having a size of approximately 2 kb is also expressed in addition to the 5-kb mRNA transcript. Especially, the gene of the present invention is differentially expressed in the normal tissues. For example, the gene of the present invention is slightly expressed in the cancer tissues and the cancer cells such as the lung cancer tissue, the metastatic lung cancer tissue, the lung cancer cell line (A549 and NCI-H358), etc., but differentially highly expressed only in the normal lung tissues. The cancer cell line into which the genes of the present invention are introduced showed a high mortality, and therefore the gene of the present invention may be effectively used for treatment and prevention of the cancer.

13. MIG 9

The gene of the present invention is a human cancer suppressor gene (MIG9) having a DNA sequence of SEQ ID NO: 49, which was deposited with Accession No. AY423724 into the GenBank database of U.S. National Institutes of Health (NIH) (Publication Date: Dec. 31, 2004), and it was revealed that the DNA sequence of the deposited gene is identical with those of the Homo sapiens S100 calcium binding protein P (S100P) gene, the Homo sapiens calcium-binding S100 protein mRNA gene and the Homo sapiens S100 calcium binding protein P gene, all deposited with Accession No. NM_{—}005980, AF539739 and BC006819 into the database, respectively. However, it was however found from this study result that the MIG9 tumor suppressor gene was slightly expressed in various human tumors including the lung cancer, while its expression was significantly increased in various normal tissues.

The DNA sequence of SEQ ID NO: 49 has one open reading frame (ORF) corresponding to base positions from 50 to 316 of the DNA sequence (base positions from 314 to 316 represent a stop codon).

The protein expressed from the gene of the present invention consists of 88 amino acid residues, and has an amino acid sequence of SEQ ID NO: 50 and a molecular weight of approximately 10 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 49. As another example, a 178-bp cDNA fragment, which is very slightly expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP12 of SEQ ID NO: 51 (5′-AAGCTTGAGTGCT-3′) and an anchored oligo-dT primer of SEQ ID NO: 52 (5′-AAGCTTTTTTTTTTTG-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, kidney, liver, placenta and peripheral bloods, to suppress carcinogenesis. The gene of the present invention is mainly overexpressed in these tissues as an mRNA transcript having a size of approximately 5 kb, and an transcript having a size of approximately 2 kb is also expressed in addition to the 5-kb mRNA transcript. Especially, the gene of the present invention is differentially expressed in the normal tissues. For example, the gene of the present invention is slightly expressed in the cancer tissues and the cancer cells such as the lung cancer tissue, the metastatic lung cancer tissue, the lung cancer cell line (A549 and NCI-H358), etc., but differentially highly expressed only in the normal lung tissues. The cancer cell line into which the genes of the present invention are introduced showed a high mortality, and therefore the gene of the present invention may be effectively used for treatment and prevention of the cancer.

14. MIG11

The gene of the present invention is a human cancer suppressor gene (MIG11) having a DNA sequence of SEQ ID NO: 53, which was deposited with Accession No. AY423726 into the GenBank database of U.S. National Institutes of Health (NIH) (Publication Date: Dec. 31, 2004), and it was revealed that the DNA sequence of the deposited gene is similar to that of the NM_{—}005943 Homo sapiens molybdenum cofactor synthesis 1 (MOCS1), transcript variant 1 gene deposited with Accession No. NM_{—}005943 into the database. However, it was however found from this study result that the MIG11 tumor suppressor gene was slightly expressed in various human tumors including the lung cancer, while its expression was significantly increased in various normal tissues.

The DNA sequence of SEQ ID NO: 53 has one open reading frame (ORF) corresponding to base positions from 7 to 756 of the DNA sequence (base positions from 754 to 756 represent a stop codon).

The protein expressed from the gene of the present invention consists of 249 amino acid residues, and has an amino acid sequence of SEQ ID NO: 54 and a molecular weight of approximately 27 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 53. As another example, a 212-bp cDNA fragment, which is very slightly expressed in the cancer tissue or the cancer cell line but differentially expressed only in the normal tissue, may be obtained by carrying out a reverse transcription-polymerase chain reaction (RT-PCR) on the total RNA extracted from a normal tissue, and a cancer tissue or a cancer cell line using a random primer H-AP13 of SEQ ID NO: 55 (5′-AAGCTTCGGCATA-3′) and an anchored oligo-dT primer of SEQ ID NO: 56 (5′-AAGCTTTTTTTTTTTA-3′), and the resultant fragment, which is used as the probe, may be plaque-hybridized with a cDNA library to obtain a full-length cDNA clone.

It is regarded that the gene of the present invention is overexpressed in the normal tissues, preferably lungs, heart, muscles, spleen, kidney, liver, placenta and peripheral blood, to suppress carcinogenesis. The gene of the present invention is mainly overexpressed in these tissues as an mRNA transcript having a size of approximately 5 kb, and an transcript having a size of approximately 2 kb is also expressed in addition to the 5-kb mRNA transcript. Especially, the gene of the present invention is differentially expressed in the normal tissues. For example, the gene of the present invention is slightly expressed in the cancer tissues and the cancer cells such as the lung cancer tissue, the metastatic lung cancer tissue, the lung cancer cell line (A549 and NCI-H358), etc., but differentially highly expressed only in the normal lung tissues. The cancer cell line into which the genes of the present invention are introduced showed a high mortality, and therefore the gene of the present invention may be effectively used for treatment and prevention of the cancer.

15. MIG15

A DNA sequence of SEQ ID NO: 57, which was deposited with Accession No. AY423730 into the GenBank database of U.S. National Institutes of Health (NIH) (Publication Date: Dec. 31, 2004), and the DNA sequencing analysis showed that the DNA sequence of the deposited gene is similar to those of the Homo sapiens degenerative spermatocyte homolog 1, lipid desaturase (Drosophila), transcript variant 1, mRNA (cDNA clone MGC:5079 IMAGE:3450936) gene; the Homo sapiens degenerative spermatocyte homolog 1, lipid desaturase (Drosophila) (DEGS1), transcript variant 1 gene; and the Homo sapiens sphingolipid delta 4 desaturase protein DES1 mRNA gene, all deposited with Accession No. BC000961, NM_{—}003676 and AF466375 into the database, respectively.

The DNA sequence of SEQ ID NO: 57 has one open reading frame (ORF) corresponding to base positions from 78 to 1049 of the DNA sequence (base positions from 1047 to 1049 represent a stop codon).

The protein expressed from the gene of the present invention consists of 323 amino acid residues, and has an amino acid sequence of SEQ ID NO: 58 and a molecular weight of approximately 38 kDa.

The gene and the protein of the present invention may be separated from human tissues, or also be synthesized according to the known methods for synthesizing DNA or peptide. For example, the gene of the present invention may be screened and cloned according to the conventional methods on the basis of the information on the DNA sequence set forth in SEQ ID NO: 57. As another example, a 327-bp cDNA fragment (corresponding to base positions from 1673 to 1999), which is not expressed in the cancer tissue or the cancer cell line but differentially expressed in the normal tissue, may be obtained by carrying out a reverse transcription-polymerase chain reaction (RT-PCR) on the total RNA extracted from a normal tissue, and a cancer tissue or a cancer cell line using a random primer H-AP31 of SEQ ID NO: 59 (5′-AAGCTTGGTGAAC-3′) and an anchored oligo-dT primer of SEQ ID NO: 60 (5′-AAGCTTTTTTTTTTTC-3′), and the resultant fragment, which is used as the probe, may be plaque-hybridized with a cDNA library to obtain a full-length cDNA clone.

Meanwhile, because of degeneracy of codons, or considering preference of codons for living organisms to express the genes, the genes of the present invention may be variously modified in coding regions without changing amino acid sequences of the proteins expressed from the coding region, and also be variously modified or changed in a region except the coding region within a range that does not affect the gene expression. Such a modified gene is also included in the scope of the present invention. Accordingly, the present invention also includes a polynucleotide having substantially the same DNA sequence as the gene; and fragments of the gene. The term “substantially the same polynucleotide” means a polynucleotide having DNA sequence homology of at least 80%, preferably at least 90%, and the most preferably at least 95%.

Also, one or more amino acids may be also substituted, added or deleted in the amino acid sequence of the protein within a range that does not affect functions of the proteins, and only some portions of the proteins may be used depending on their usage. Such a modified amino acid sequence is also included in the scope of the present invention. Accordingly, the present invention also includes a polypeptide having substantially the same amino acid sequence as the protein; and fragments of the protein. The term “substantially the same polypeptide” means a polypeptide having sequence homology of at least 80%, preferably at least 90%, and the most preferably at least 95%.

The genes prepared thus may be inserted into each vector for expression in microorganisms or animal cells, already known in the art, to obtain expression vectors, and then DNA of the genes may be replicated in a large quantity or its protein may be produced in a commercial quantity by introducing each of the expression vectors into suitable host cells, for example Escherichia coli, a Hela cell line, etc. Upon constructing the expression vector, DNA regulatory sequences such as a promoter and a terminator, autonomously replicating sequences, secretion signals, etc. may be suitably selected and combined depending on kinds of the host cells that produce the gene or the protein.

It is regarded that the gene of the present invention is overexpressed in the normal tissues, preferably uterus, heart, skeletal muscles, thymus, spleen, kidney, liver, small intestines, placenta and peripheral blood leukocyte, to suppress carcinogenesis. The gene of the present invention is mainly overexpressed in these tissues as an mRNA transcript having a size of approximately 9.5 kb. Especially, the gene of the present invention is differentially expressed only in the normal tissues. For example, the gene of the present invention is not expressed or slightly expressed in the cancer tissues and the cancer cells such as the uterine cancer tissue and the uterine cancer cell line, but differentially highly expressed only in the normal uterine tissues. The cancer cell line into which the genes of the present invention are introduced showed a high mortality, and therefore the gene of the present invention may be effectively used for treatment and prevention of the cancer.

Hereinafter, the present invention will be described in detail referring to preferred examples. Therefore, the description proposed herein is just a preferable example for the purpose of illustrations only, not intended to limit the scope of the invention.

### REFERENCE EXAMPLE

### Separation of Total RNA

The total RNA samples were separated from fresh tissues or cultured cells using the RNeasy total RNA kit (Qiagen Inc., Germany), and then the contaminated DNA was removed from the RNA samples using the message clean kit (GenHunter Corp., MA, U.S.).

### Example 1

### Separation of Total RNA and Differential Display of mRNA

1-1. GIG1

A differential expression pattern of the gene of interest was measured in a normal exocervical tissue, a primary cervical cancer tissue and an cervical cancer cell line, as follows. A normal exocervical tissue sample was obtained from a patient suffering from an uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 4 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP38 (RNAimage primer set 5, GenHunter Corporation, U.S.) of SEQ ID NO: 3. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

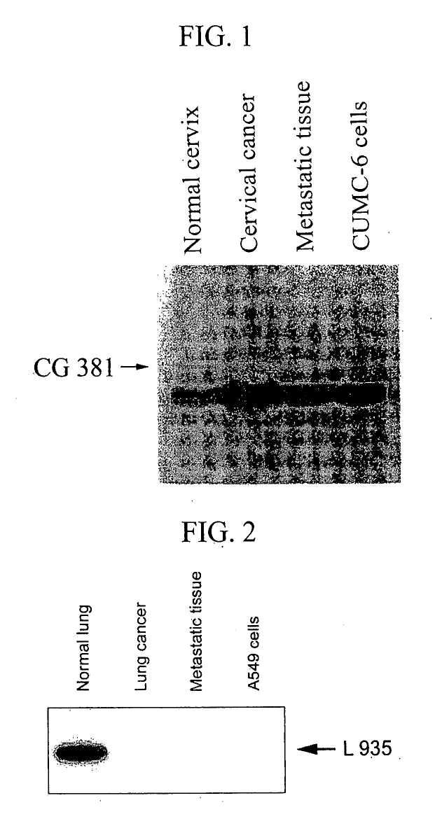

FIG. 1 shows a PCR result using a random 5′13-mer primer H-AP38 of SEQ ID NO: 3 and an anchored oligo-dT primer of SEQ ID NO: 4. In FIG. 1, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 1, it was confirmed that a 382-bp cDNA fragment was not expressed in the cervical cancer tissue, the metastatic iliac lymph node tissue and the cervical cancer cell line CUMC-6, but differentially expressed only in the normal exocervical tissue. The cDNA fragment was named CG381.

A 382-bp band, CG381 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment CG381 was cloned into an expression vector pGEM-T Easy using the TA cloning system (Promega), and then its DNA sequence was determined using the Sequenase Version 2.0 DNA Sequencing System (United States Biochemical Co.).

1-2. GIG3

A differential expression pattern of the gene of interest was measured in a normal lung tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 8 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP32 (RNAimage primer set 1, GenHunter Corporation, U.S.) of SEQ ID NO: 7. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 2 shows a PCR result using a random 5′13-mer primer H-AP32 of SEQ ID NO: 7 and an anchored oligo-dT primer of SEQ ID NO: 8. In FIG. 2, Lane 1 represents the normal lung tissue; Lane 2 represents the lung cancer tissue; Lane 3 represents the metastatic lung cancer tissue; Lane 4 represents the lung cancer cell line A549. As shown in FIG. 2, it was confirmed that a 190-bp cDNA fragment was not expressed in the lung cancer tissue, the metastatic lung cancer tissue and the lung cancer cell line, but differentially expressed only in the normal lung tissue (Base positions from 492 to 681 of the full-length GIG3 gene sequence). The cDNA fragment was named L935.

A 190-bp band, L935 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L935 was cloned into an expression vector pGEM-T Easy using the TA cloning system (Promega), and then its DNA sequence was determined using the Sequenase Version 2.0 DNA Sequencing System (United States Biochemical Co.).

1-3 GIG4

A normal lung tissue sample, a lung cancer tissue sample and a metastatic lung cancer tissue sample were obtained from a lung cancer patient during surgical operation in the same manner as described in Example 1-2. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 12 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP35 (RNAimage primer set 1, GenHunter Corporation, U.S.) of SEQ ID NO: 11. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

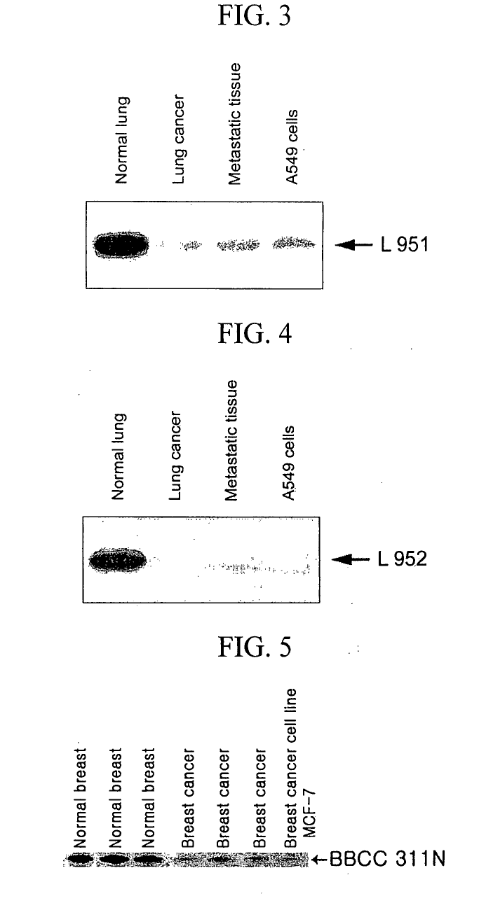

FIG. 3 shows a PCR result using a random 5′13-mer primer H-AP35 of SEQ ID NO: 11 and an anchored oligo-dT primer of SEQ ID NO: 12. In FIG. 3, Lane 1 represents the normal lung tissue; Lane 2 represents the lung cancer tissue; Lane 3 represents the metastatic lung cancer tissue; Lane 4 represents the lung cancer cell line A549. As shown in FIG. 3, it was confirmed that a 187-bp cDNA fragment was slightly expressed in the lung cancer tissue, the metastatic lung cancer tissue and the lung cancer cell line, but differentially expressed only in the normal lung tissue (Base positions from 435 to 621 of the full-length GIG4 gene sequence). The cDNA fragment was named L951.

A 187-bp band, L951 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L951 was cloned into an expression vector pGEM-T Easy using the TA cloning system (Promega), and then its DNA sequence was determined using the Sequenase Version 2.0 DNA Sequencing System (United States Biochemical Co.).

1-4 GIG5

A differential expression pattern of the gene of interest was measured in a normal lungs tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 16 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP32 (RNAimage primer set 1, GenHunter Corporation, U.S.) of SEQ ID NO: 15. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 4 shows a PCR result using a random 5′13-mer primer H-AP34 of SEQ ID NO: 15 and an anchored oligo-dT primer of SEQ ID NO: 16. In FIG. 4, Lane 1 represents the normal lung tissue; Lane 2 represents the lung cancer tissue; Lane 3 represents the metastatic lung cancer tissue; Lane 4 represents the lung cancer cell line A549. As shown in FIG. 4, it was confirmed that a 212-bp cDNA fragment was not expressed in the lung cancer tissue, the metastatic lung cancer tissue and the lung cancer cell line, but differentially expressed only in the normal lung tissue (Base positions from 497 to 708 of the full-length GIG5 gene sequence). The cDNA fragment was named L952.

A 212-bp band, L952 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L952 was cloned into an expression vector pGEM-T Easy using the TA cloning system (Promega), and then its DNA sequence was determined using the Sequenase Version 2.0 DNA Sequencing System (United States Biochemical Co.).

1-5: GIG11

A differential expression pattern of the gene of interest was measured in a normal breast tissue, a primary breast cancer tissue and a breast cancer cell line, as follows.

A normal breast tissue sample was obtained from a breast cancer patient during mastectomy, and a primary breast cancer tissue was obtained during radical mastectomy from patient suffering from the uterine cancer who has not been subject to the radiation and/or anticancer therapies before surgical operation. MCF-7 (American Type Culture Collection; ATCC Number HTB-22) was used as the human breast cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 20 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP36 (RNAimage primer set 5, GenHunter Corporation, U.S.) of SEQ ID NO: 19. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 5 shows a PCR result using a random 5′13-mer primer H-AP36 of SEQ ID NO: 3 and an anchored oligo-dT primer of SEQ ID NO: 4. In FIG. 5, Lanes 1, 2 and 3 represent the normal breast tissue; Lanes 4, 5 and 6 represent the breast cancer tissue; and Lane 7 represents the breast cancer cell line MCF-7. As shown in FIG. 5, it was confirmed that a 298-bp cDNA fragment was very slightly expressed in the breast cancer tissue and the breast cancer cell line, but differentially expressed only in the normal breast tissue (Base positions from 741 to 1038 of the full-length GIG11 gene sequence). The cDNA fragment was named BBCC311N.

A 298-bp band, BBCC311N fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment BBCC311N was cloned into an expression vector pGEM-T Easy using the TA cloning system (Promega), and then its DNA sequence was determined using the Sequenase Version 2.0 DNA Sequencing System (United States Biochemical Co.).

1-6: MIG2

A differential expression pattern of the gene of interest was measured in a normal exocervical tissue, a primary cervical cancer tissue and an cervical cancer cell line, as follows. A normal exocervical tissue sample was obtained from a patient suffering from the uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 24 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP35 (RNAimage primer set 5, GenHunter Corporation, U.S.) of SEQ ID NO: 23. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

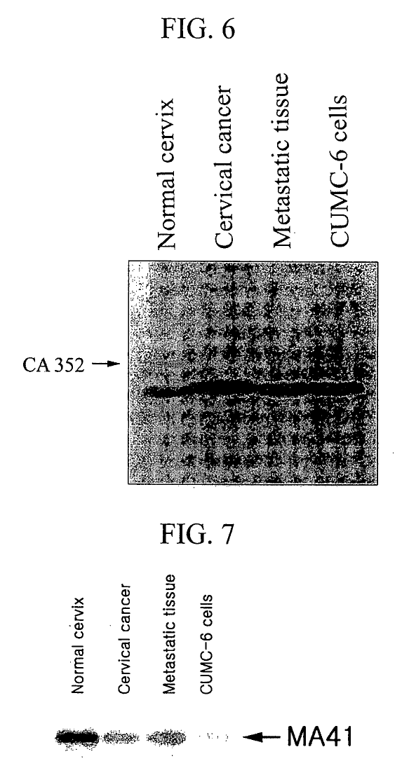

FIG. 6 shows a PCR result using a random 5′13-mer primer H-AP35 of SEQ ID NO: 23 and an anchored oligo-dT primer of SEQ ID NO: 24. In FIG. 6, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 6, it was confirmed that a 311-bp cDNA fragment was not expressed in the cervical cancer tissue, the metastatic iliac lymph node tissue and the cervical cancer cell line CUMC-6, but differentially expressed only in the normal exocervical tissue. The cDNA fragment was named CA352.

A 311-bp band, CA352 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment CA352 was cloned into an expression vector pGEM-T Easy using the TA cloning system (Promega), and then its DNA sequence was determined using the Sequenase Version 2.0 DNA Sequencing System (United States Biochemical Co.).

1-7: MIG4

A differential expression pattern of the gene of interest was measured in a normal exocervical tissue, a primary cervical cancer tissue and an cervical cancer cell line, as follows. A normal exocervical tissue sample was obtained from a patient suffering from the uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 28 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP31 (RNAimage primer set 5, GenHunter Corporation, U.S.) of SEQ ID NO: 27. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 7 shows a PCR result using a random 5′13-mer primer H-AP31 of SEQ ID NO: 27 and an anchored oligo-dT primer of SEQ ID NO: 28. In FIG. 7, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 7, it was confirmed that a 322-bp cDNA fragment was not expressed in the cervical cancer tissue, the metastatic iliac lymph node tissue and the cervical cancer cell line CUMC-6, but differentially expressed only in the normal exocervical tissue. The cDNA fragment was named MA41.

A 322-bp band, MA41 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment MA41 was cloned into an expression vector pGEM-T Easy using the TA cloning system (Promega), and then its DNA sequence was determined using the Sequenase Version 2.0 DNA Sequencing System (United States Biochemical Co.).

1-8: PIG13

A differential expression pattern of the gene of interest was measured in a normal lungs tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 32 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP6 (RNAimage primer set 1, GenHunter Corporation, U.S.) of SEQ ID NO: 31. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

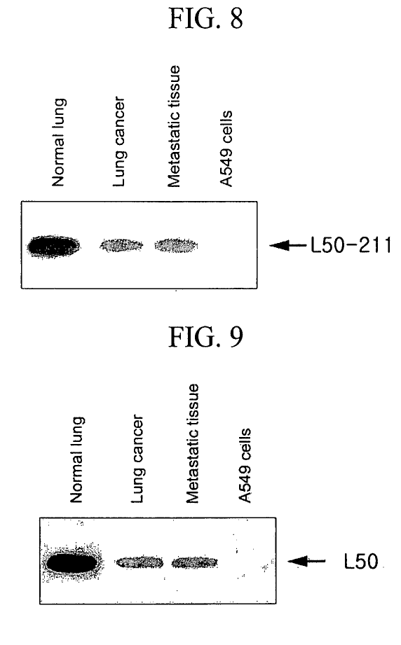

FIG. 8 shows a PCR result using a random 5′13-mer primer H-AP6 of SEQ ID NO: 31 and an anchored oligo-dT primer of SEQ ID NO: 32. In FIG. 8, Lanes 1 represents the normal lung tissue; Lanes 2 represents the lung cancer tissue; Lane 3 represents the metastatic lung cancer tissue; Lane 4 represents the lung cancer cell line A549. As shown in FIG. 8, it was confirmed that a 296-bp cDNA fragment was not expressed or slightly expressed in the lung cancer tissue, the metastatic lung cancer tissue and the lung cancer cell line, but differentially expressed only in the normal lung tissue (Base positions from 643 to 938 of the full-length PIG13 gene sequence). The cDNA fragment was named L50-211.

A 296-bp band, L50-211 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L50-211 was cloned into an expression vector pGEM-T Easy using the TA cloning system (Promega), and then its DNA sequence was determined using the Sequenase Version 2.0 DNA Sequencing System (United States Biochemical Co.).

1-9: PIG15

A differential expression pattern of the gene of interest was measured in a normal lung tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 36 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP6 (RNAimage primer set 1, GenHunter Corporation, U.S.) of SEQ ID NO: 35. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

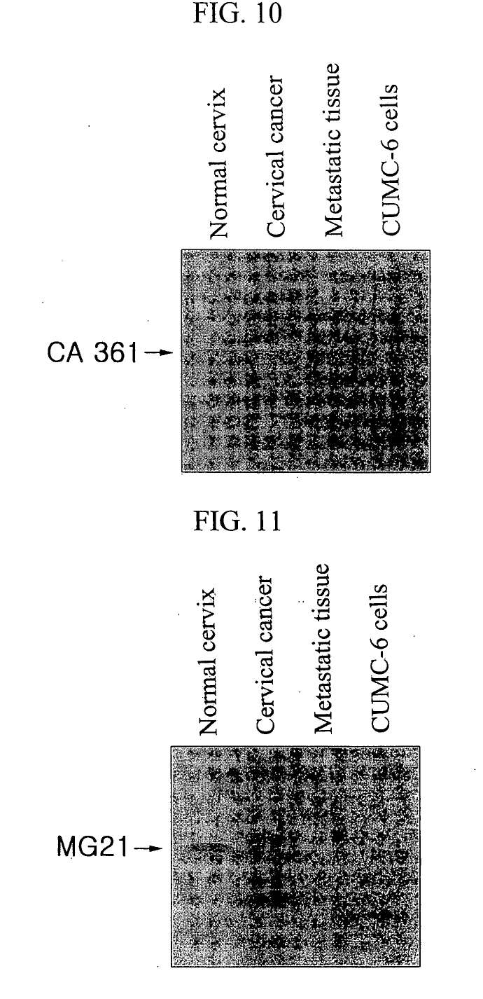

FIG. 9 shows a PCR result using a random 5′13-mer primer H-AP6 of SEQ ID NO: 35 and an anchored oligo-dT primer of SEQ ID NO: 36. In FIG. 9, Lanes 1 represents the normal lung tissue; Lanes 2 represents the lung cancer tissue; Lane 3 represents the metastatic lung cancer tissue; Lane 4 represents the lung cancer cell line A549. As shown in FIG. 9, it was confirmed that a 327-bp cDNA fragment was not expressed or slightly expressed in the lung cancer tissue, the metastatic lung cancer tissue and the lung cancer cell line, but differentially expressed only in the normal lung tissue (Base positions from 1373 to 1699 of the full-length PIG15 gene sequence). The cDNA fragment was named L50.

A 327-bp band, L50 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L50 was cloned into an expression vector pGEM-T Easy using the TA cloning system (Promega), and then its DNA sequence was determined using the Sequenase Version 2.0 DNA Sequencing System (United States Biochemical Co.).

1-10: PIG8

A differential expression pattern of the gene of interest was measured in a normal exocervical tissue, a primary cervical cancer tissue and an cervical cancer cell line, as follows. A normal exocervical tissue sample was obtained from a patient suffering from the uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 40 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP36 (RNAimage primer set 5, GenHunter Corporation, U.S.) of SEQ ID NO: 39. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 10 shows a PCR result using a random 5′13-mer primer H-AP36 of SEQ ID NO: 39 and an anchored oligo-dT primer of SEQ ID NO: 40. In FIG. 10, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 10, it was confirmed that a 362-bp cDNA fragment was not expressed in the cervical cancer tissue, the metastatic iliac lymph node tissue and the cervical cancer cell line CUMC-6, but differentially expressed only in the normal exocervical tissue. The cDNA fragment was named CA361.

A 362-bp band, CG361 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment CG361 was cloned into an expression vector pGEM-T Easy using the TA cloning system (Promega), and then its DNA sequence was determined using the Sequenase Version 2.0 DNA Sequencing System (United States Biochemical Co.).

1-11: MRG1

A differential expression pattern of the gene of interest was measured in a normal exocervical tissue, a primary cervical cancer tissue and an cervical cancer cell line, as follows. A normal exocervical tissue sample was obtained from a patient suffering from the uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 44 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP21 (RNAimage primer set 5, GenHunter Corporation, U.S.) of SEQ ID NO: 43. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 11 shows a PCR result using a random 5′13-mer primer H-AP21 of SEQ ID NO: 43 and an anchored oligo-dT primer of SEQ ID NO: 44. In FIG. 11, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 11, it was confirmed that a 277-bp cDNA fragment was not expressed in the cervical cancer tissue, the metastatic iliac lymph node tissue and the cervical cancer cell line CUMC-6, but differentially expressed only in the normal exocervical tissue. The cDNA fragment was named MG21.

A 277-bp band, MG21 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment MG21 was cloned into an expression vector pGEM-T Easy using the TA cloning system (Promega), and then its DNA sequence was determined using the Sequenase Version 2.0 DNA Sequencing System (United States Biochemical Co.).

1-12: PIG22

A differential expression pattern of the gene of interest was measured in a normal lung tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 48 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP24 (RNAimage primer set 1, GenHunter Corporation, U.S.) of SEQ ID NO: 47. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

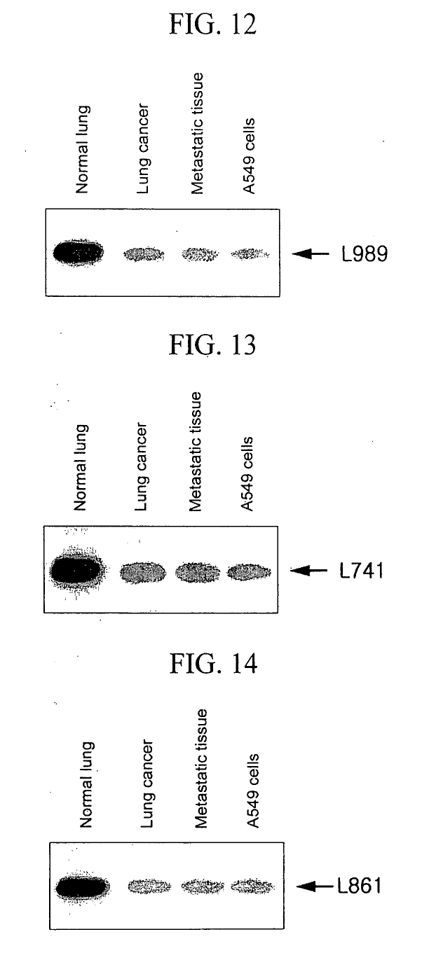

FIG. 12 shows a PCR result using a random 5′13-mer primer H-AP24 of SEQ ID NO: 47 and an anchored oligo-dT primer of SEQ ID NO: 48. In FIG. 12, Lanes 1 represents the normal lung tissue; Lanes 2 represents the lung cancer tissue; Lane 3 represents the metastatic lung cancer tissue; Lane 4 represents the lung cancer cell line A549. As shown in FIG. 12, it was confirmed that a 242-bp cDNA fragment was slightly expressed in the lung cancer tissue, the metastatic lung cancer tissue and the lung cancer cell line, but differentially expressed only in the normal lung tissue (Base positions from 738 to 979 of the full-length PIG22 gene sequence). The cDNA fragment was named L989.

A 242-bp band, L989 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L989 was cloned into an expression vector pGEM-T Easy using the TA cloning system (Promega), and then its DNA sequence was determined using the Sequenase Version 2.0 DNA Sequencing System (United States Biochemical Co.).

1-13: MIG9

A differential expression pattern of the gene of interest was measured in a normal lung tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 52 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP12 (RNAimage primer set 1, GenHunter Corporation, U.S.) of SEQ ID NO: 51. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 13 shows a PCR result using a random 5′13-mer primer H-AP12 of SEQ ID NO: 51 and an anchored oligo-dT primer of SEQ ID NO: 52. In FIG. 13, Lanes 1 represents the normal lung tissue; Lanes 2 represents the lung cancer tissue; Lane 3 represents the metastatic lung cancer tissue; Lane 4 represents the lung cancer cell line A549. As shown in FIG. 13, it was confirmed that a 178-bp cDNA fragment was slightly expressed in the lung cancer tissue, the metastatic lung cancer tissue and the lung cancer cell line, but differentially expressed only in the normal lung tissue (Base positions from 132 to 309 of the full-length MIG9 gene sequence). The cDNA fragment was named L741.

A 178-bp band, L741 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L741 was cloned into an expression vector pGEM-T Easy using the TA cloning system (Promega), and then its DNA sequence was determined using the Sequenase Version 2.0 DNA Sequencing System (United States Biochemical Co.).

1-14: MIG11

A differential expression pattern of the gene of interest was measured in a normal lung tissue, a primary lung cancer tissue, metastatic lung cancer tissue and a lung cancer cell line, as follows. A normal lung tissue sample, a lung cancer tissue sample and a metastatic lung cancer tissue sample were obtained from a lung cancer patient during surgical operation. A549 (American Type Culture Collection; ATCC Number CCL-185) and NCI-H358 (American Type Culture Collection; ATCC Number CRL-5807) were used as the human lung cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 56 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP13 (RNAimage primer set 1, GenHunter Corporation, U.S.) of SEQ ID NO: 55. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

FIG. 14 shows a PCR result using a random 5′13-mer primer H-AP13 of SEQ ID NO: 55 and an anchored oligo-dT primer of SEQ ID NO: 56. In FIG. 14, Lanes 1 represents the normal lung tissue; Lanes 2 represents the lung cancer tissue; Lane 3 represents the metastatic lung cancer tissue; Lane 4 represents the lung cancer cell line A549. As shown in FIG. 14, it was confirmed that a 212-bp cDNA fragment was slightly expressed in the lung cancer tissue, the metastatic lung cancer tissue and the lung cancer cell line, but differentially expressed only in the normal lung tissue (Base positions from 568 to 779 of the full-length MIG11 gene sequence). The cDNA fragment was named L861.

A 212-bp band, L861 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment L861 was cloned into an expression vector pGEM-T Easy using the TA cloning system (Promega), and then its DNA sequence was determined using the Sequenase Version 2.0 DNA Sequencing System (United States Biochemical Co.).

1-15: MIG15

A differential expression pattern of the gene of interest was measured in a normal exocervical tissue, a primary cervical cancer tissue and an cervical cancer cell line, as follows. A normal exocervical tissue sample was obtained from a patient suffering from the uterine myoma during hysterectomy, and a primary cervical tumor tissue sample and a metastatic iliac lymph node tumor tissue sample were obtained during radical hysterectomy from patient suffering from the uterine cancer who has not been subject to the radiation and/or anticancer therapies before surgical operation. CUMC-6 (Kim, J. W. et al., Gynecol. Oncol. 62: 230-240, 1996) was used as the human cervical cancer cell line. The total RNA samples were separated from these tissues and cells in the same manner as described in the reference example.

A RT-PCR reaction was carried out using each of the total RNA samples separated from the tissues and the cells according to a modified method as described in the disclosure (Liang, P. and Pardee, A. B., Science, 257, 967-971 (1992); and Liang, P. et al., Cancer Res., 52, 6966-6968 (1993)), as follows. 0.2 μg of the total RNA was reverse-transcribed with an anchored oligo-dT primer of SEQ ID NO: 60 using a kit (a RNAimage kit, GenHunter), and then a PCR reaction was carried out in the presence of 0.5 mM [α-³⁵S]-labeled dATP (1,200 Ci/mmol) using the same anchored oligo-dT primer and a random 5′13-mer primer H-AP31 (RNAimage primer set 5, GenHunter Corporation, U.S.) of SEQ ID NO: 59. The PCR reaction was conducted under the following conditions: the total 40 amplification cycles consisting of a denaturation step at 95° C. for 40 seconds, an annealing step at 40° C. for 2 minutes and an extension step at 72° C. for 40 seconds, and followed by one extension step at 72° C. for 5 minutes. The amplified fragments were electrophoresized in a 6% polyacrylamide gel for DNA sequencing, and then autoradiographed.

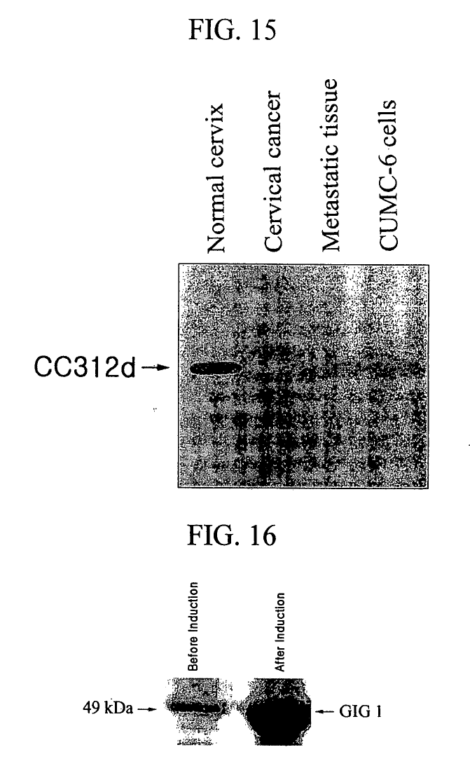

FIG. 15 shows a PCR result using a random 5′13-mer primer H-AP31 of SEQ ID NO: 59 and an anchored oligo-dT primer of SEQ ID NO: 60. In FIG. 15, Lane 1 represents the normal exocervical tissue; Lane 2 represents the cervical cancer tissue; Lane 3 represents the metastatic iliac lymph node tissue; Lane 4 represents the cervical cancer cell line CUMC-6. As shown in FIG. 15, it was confirmed that a 327-bp cDNA fragment was slightly expressed in the cervical cancer tissue, the metastatic iliac lymph node tissue and the cervical cancer cell line CUMC-6, but differentially expressed only in the normal exocervical tissue. The cDNA fragment was named CC312.

A 327-bp band, CC312 fragment, was removed from the dried gell, boiled for 15 minutes to elute cDNA, and a PCR reaction was then carried out under the same said condition using the same primer set as described above to re-amplify the cDNA, except that the [α-³⁵S]-labeled dATP and the 20 μM dNTP were not used herein. The re-amplified cDNA fragment CC312 was cloned into an expression vector pGEM-T Easy using the TA cloning system (Promega), and then its DNA sequence was determined using the Sequenase Version 2.0 DNA Sequencing System (United States Biochemical Co.).

### Example 2

### cDNA Library Screening

2-1: GIG1

The cDNA fragment CG381 obtained in Example 1-1 was labeled according to the method of the disclosure (Feinberg, A. P. and Vogelstein, B., Anal. Biochem., 132, 6-13 (1983)) to obtain a ³²P-labeled FC26 cDNA probe, and the ³²P-labeled FC26 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene GIG1.

The full-length cDNA was sequenced, and therefore its DNA sequence was identical with SEQ ID NO: 1. The cDNA sequence has an open reading frame encoding 320 amino acid residues, and the amino acid sequence derived from the open reading frame was identical with SEQ ID NO: 2. The derived protein also had a molecular weight of approximately 34 kDa.

The resultant full-length GIG1 cDNA clone was inserted into a multi-cloning site of the prokaryotic expression vector pBAD/thio-Topo (Invitrogen, U.S.) to obtain a vector pBAD/thio-Topo/GIG1, and Escherichia coli Top10 (Invitrogen, U.S.) was then transformed with the resultant pBAD/thio-Topo/GIG1. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate. FIG. 16 is a diagram showing an expression pattern of proteins of the E. coli Top10 strain transformed with the vector pBAD/thio-Topo/GIG1 using the SDS-PAGE, and it was revealed that a band of a fusion protein having a molecular weight of approximately 49 kDa was clearly observed after the L-arabinose induction. The 49-kDa fusion protein corresponds to a protein including the approximately 15-kDa HT-thioredoxin protein inserted into the vector pBAD/thio-Topo/GIG1, and the approximately 34-kDa GIG1 protein.

FIG. 16 is a diagram showing an SDS-PAGE analysis of the GIG1 protein. In FIG. 16, Lane 1 represents a protein sample before the L-arabinose induction, and Lane 2 represents a protein sample after the L-arabinose induction.

2-2: GIG3

The cDNA fragment L935 obtained in Example 1-2 was labeled according to the method of the disclosure (Feinberg, A. P. and Vogelstein, B., Anal. Biochem., 132, 6-13 (1983)) to obtain a ³²P-labeled L935 cDNA probe, and the ³²P-labeled L935 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene GIG3.

The full-length cDNA was sequenced, and therefore its DNA sequence was identical with SEQ ID NO: 5. The cDNA sequence has an open reading frame encoding 208 amino acid residues, and the amino acid sequence derived from the open reading frame was identical with SEQ ID NO: 6. The derived protein also had a molecular weight of approximately 23 kDa.

The transformed E. coli strain was incubated in LB broth, and then 1 mM isopropy-1-β-D-thiogalactopyranoside (IPTG) was added to the culture, and reacted at 37° C. for 3 hours to express the GIG2 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

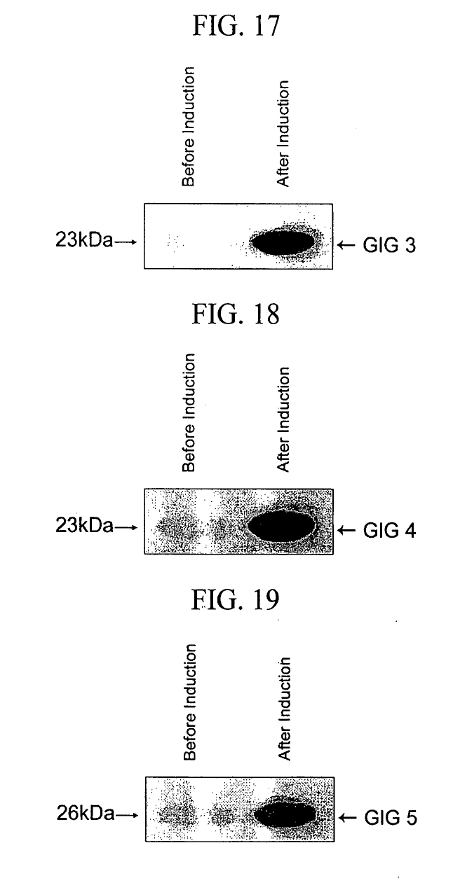

FIG. 17 is a diagram showing an SDS-PAGE analysis of the GIG3 protein. In FIG. 17, Lane 1 represents a protein sample before the IPTG induction, and Lane 2 represents a protein sample after expression of the GIG3 gene is induced by IPTG. As shown in FIG. 17, the expressed GIG3 protein has a molecular weight of approximately 23 kDa, which corresponds to a molecular weight of a protein derived from its DNA sequence.

2-3: GIG4

The cDNA fragment L951 obtained in Example 1-3 was labeled according to the method of the disclosure (Feinberg, A. P. and Vogelstein, B., Anal. Biochem., 132, 6-13 (1983)) to obtain a ³²P-labeled L951 cDNA probe, and the ³²P-labeled L951 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene GIG4.

The full-length cDNA was sequenced, and therefore its DNA sequence was identical with SEQ ID NO: 9. The cDNA sequence has an open reading frame encoding 203 amino acid residues, and the amino acid sequence derived from the open reading frame was identical with SEQ ID NO: 10. The derived protein also had a molecular weight of approximately 23 kDa.

The transformed E. coli strain was incubated in LB broth, and then 1 mM isopropy-1-β-D-thiogalactopyranoside (IPTG) was added to the culture, and reacted at 37° C. for 3 hours to express the GIG4 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

FIG. 18 is a diagram showing an SDS-PAGE analysis of the GIG4 protein. In FIG. 18, Lane 1 represents a protein sample before the IPTG induction, and Lane 2 represents a protein sample after expression of the GIG4 gene is induced by IPTG. As shown in FIG. 18, the expressed GIG4 protein has a molecular weight of approximately 23 kDa, which corresponds to a molecular weight of a protein derived from its DNA sequence.

2-4: GIG5

The cDNA fragment L952 obtained in Example 1-4 was labeled according to the method of the disclosure (Feinberg, A. P. and Vogelstein, B., Anal. Biochem., 132, 6-13 (1983)) to obtain a ³²P-labeled L952 cDNA probe, and the ³²P-labeled L952 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene GIG5.

The full-length cDNA was sequenced, and therefore its DNA sequence was identical with SEQ ID NO: 13. The cDNA sequence has an open reading frame encoding 228 amino acid residues, and the amino acid sequence derived from the open reading frame was identical with SEQ ID NO: 14. The derived protein also had a molecular weight of approximately 26 kDa.

The transformed E. coli strain was incubated in LB broth, and then 1 mM isopropy-1-β-D-thiogalactopyranoside (IPTG) was added to the culture, and reacted at 37° C. for 3 hours to express the GIG5 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

FIG. 19 is a diagram showing an SDS-PAGE analysis of the GIG5 protein. In FIG. 19, Lane 1 represents a protein sample before the IPTG induction, and Lane 2 represents a protein sample after expression of the GIG5 gene is induced by IPTG. As shown in FIG. 19, the expressed GIG5 protein has a molecular weight of approximately 26 kDa, which corresponds to a molecular weight of a protein derived from its DNA sequence.

2-5: GIG11

The cDNA fragment BBCC311N obtained in Example 1-5 was labeled according to the method of the disclosure (Feinberg, A. P. and Vogelstein, B., Anal. Biochem., 132, 6-13 (1983)) to obtain a ³²P-labeled BBCC311N cDNA probe, and the ³²P-labeled BBCC311N cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene GIG1.

The full-length cDNA was sequenced, and therefore its DNA sequence was identical with SEQ ID NO: 17. The cDNA sequence has an open reading frame encoding 250 amino acid residues, and the amino acid sequence derived from the open reading frame was identical with SEQ ID NO: 18. The derived protein also had a molecular weight of approximately 29 kDa.

The transformed E. coli strain was incubated in LB broth, and then 1 mM isopropy-1-β-D-thiogalactopyranoside (IPTG) was added to the culture, and reacted at 37° C. for 3 hours to express the GIG11 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

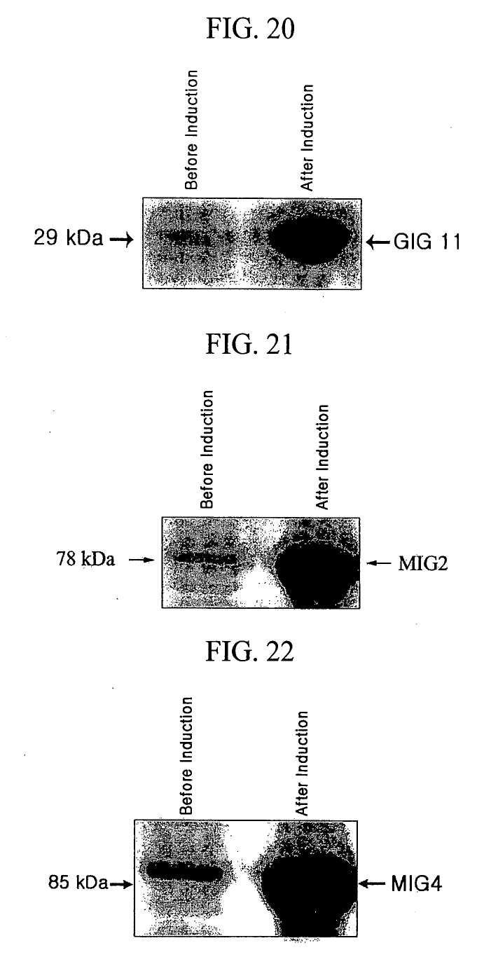

FIG. 20 is a diagram showing an SDS-PAGE analysis of the GIG11 protein. In FIG. 20, Lane 1 represents a protein sample before the IPTG induction, and Lane 2 represents a protein sample after expression of the GIG11 gene is induced by IPTG. As shown in FIG. 20, the expressed GIG11 protein has a molecular weight of approximately 29 kDa, which corresponds to a molecular weight of a protein derived from its DNA sequence.

2-6: MIG2

The cDNA fragment CA352 obtained in Example 1-6 was labeled according to the method of the disclosure (Feinberg, A. P. and Vogelstein, B., Anal. Biochem., 132, 6-13 (1983)) to obtain a ³²P-labeled FC26 cDNA probe, and the ³²P-labeled FC26 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MIG2.

The full-length cDNA was sequenced, and therefore its DNA sequence was identical with SEQ ID NO: 21. The cDNA sequence has an open reading frame encoding 578 amino acid residues, and the amino acid sequence derived from the open reading frame was identical with SEQ ID NO: 22. The derived protein also had a molecular weight of approximately 63 kDa.

The resultant full-length MIG2 cDNA clone was inserted into a multi-cloning site of the prokaryotic expression vector pBAD/thio-Topo (Invitrogen, U.S.) to obtain a vector pBAD/thio-Topo/MIG2, and Escherichia coli Top10 (Invitrogen, U.S.) was then transformed with the resultant pBAD/thio-Topo/MIG2. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate.

FIG. 21 is a diagram showing an expression pattern of proteins of the E. coli Top10 strain transformed with the vector pBAD/thio-Topo/MIG2 using the SDS-PAGE, and it was revealed that a band of a fusion protein having a molecular weight of approximately 78 kDa was clearly observed after the L-arabinose induction. The 78-kDa fusion protein corresponds to a protein including the approximately 15-kDa HT-thioredoxin protein inserted into the vector pBAD/thio-Topo/MIG2, and the approximately 63-kDa MIG1 protein.

FIG. 21 is a diagram showing an SDS-PAGE analysis of the MIG2 protein. In FIG. 21, Lane 1 represents a protein sample before the L-arabinose induction, and Lane 2 represents a protein sample after expression of the MIG2 gene is induced by L-arabinose.

2-7: MIG4

The cDNA fragment MA41 obtained in Example 1-7 was labeled according to the method of the disclosure (Feinberg, A. P. and Vogelstein, B., Anal. Biochem., 132, 6-13 (1983)) to obtain a ³²P-labeled FC26 cDNA probe, and the ³²P-labeled FC26 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MIG4.

The full-length cDNA was sequenced, and therefore its DNA sequence was identical with SEQ ID NO: 25. The cDNA sequence has an open reading frame encoding 640 amino acid residues, and the amino acid sequence derived from the open reading frame was identical with SEQ ID NO: 26. The derived protein also had a molecular weight of approximately 70 kDa.

The resultant full-length MIG4 cDNA clone was inserted into a multi-cloning site of the prokaryotic expression vector pBAD/thio-Topo (Invitrogen, U.S.) to obtain a vector pBAD/thio-Topo/MIG4, and Escherichia coli Top10 (Invitrogen, U.S.) was then transformed with the resultant pBAD/thio-Topo/MIG4. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate. FIG. 22 is a diagram showing an expression pattern of proteins of the E. coli Top10 strain transformed with the vector pBAD/thio-Topo/MIG4 using the SDS-PAGE, and it was revealed that a band of a fusion protein having a molecular weight of approximately 85 kDa was clearly observed after the L-arabinose induction. The 85-kDa fusion protein corresponds to a protein including the approximately 15-kDa HT-thioredoxin protein inserted into the vector pBAD/thio-Topo/MIG4, and the approximately 70-kDa MIG4 protein.

FIG. 22 is a diagram showing an SDS-PAGE analysis of the MIG4 protein. In FIG. 22, Lane 1 represents a protein sample before the L-arabinose induction, and Lane 2 represents a protein sample after expression of the MIG4 gene is induced by L-arabinose.

2-8: PIG13

The cDNA fragment L50-211 obtained in Example 1-8 was labeled according to the method of the disclosure (Feinberg, A. P. and Vogelstein, B., Anal. Biochem., 132, 6-13 (1983)) to obtain a ³²P-labeled L50-211 cDNA probe, and the ³²P-labeled L50-211 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene PIG13.

The full-length cDNA was sequenced, and therefore its DNA sequence was identical with SEQ ID NO: 29. The cDNA sequence has an open reading frame encoding 121 amino acid residues, and the amino acid sequence derived from the open reading frame was identical with SEQ ID NO: 30. The derived protein also had a molecular weight of approximately 14 kDa.

The transformed E. coli strain was incubated in LB broth, and then 1 mM isopropy-1-β-D-thiogalactopyranoside (IPTG) was added to the culture, and reacted at 37° C. for 3 hours to express the PIG13 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

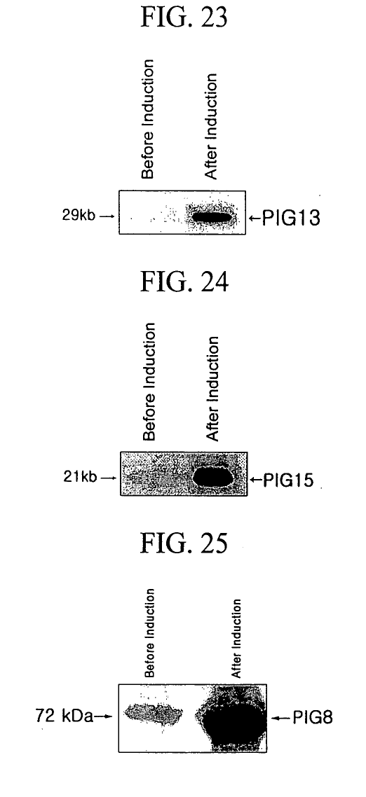

FIG. 23 is a diagram showing an SDS-PAGE analysis of the PIG13 protein. In FIG. 23, Lane 1 represents a protein sample before the IPTG induction, and Lane 2 represents a protein sample after expression of the PIG13 gene is induced by IPTG. As shown in FIG. 23, the expressed PIG13 protein has a molecular weight of approximately 14 kDa, which corresponds to a molecular weight of a protein derived from its DNA sequence.

2-9: PIG15

The cDNA fragment L50 obtained in Example 1-9 was labeled according to the method of the disclosure (Feinberg, A. P. and Vogelstein, B., Anal. Biochem., 132, 6-13 (1983)) to obtain a ³²P-labeled L50 cDNA probe, and the ³²P-labeled L50 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene PIG15.

The full-length cDNA was sequenced, and therefore its DNA sequence was identical with SEQ ID NO: 33. The cDNA sequence has an open reading frame encoding 183 amino acid residues, and the amino acid sequence derived from the open reading frame was identical with SEQ ID NO: 34. The derived protein also had a molecular weight of approximately 21 kDa.

The transformed E. coli strain was incubated in LB broth, and then 1 mM isopropy-1-β-D-thiogalactopyranoside (IPTG) was added to the culture, and reacted at 37° C. for 3 hours to express the PIG15 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

FIG. 24 is a diagram showing an SDS-PAGE analysis of the PIG15 protein. In FIG. 24, Lane 1 represents a protein sample before the IPTG induction, and Lane 2 represents a protein sample after expression of the PIG15 gene is induced by IPTG. As shown in FIG. 24, the expressed PIG15 protein has a molecular weight of approximately 21 kDa, which corresponds to a molecular weight of a protein derived from its DNA sequence.

2-10: PIG8

The cDNA fragment CA361 obtained in Example 1-10 was labeled according to the method of the disclosure (Feinberg, A. P. and Vogelstein, B., Anal. Biochem., 132, 6-13 (1983)) to obtain a ³²P-labeled FC26 cDNA probe, and the ³²P-labeled FC26 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene PIG8.

The full-length cDNA was sequenced, and therefore its DNA sequence was identical with SEQ ID NO: 37. The cDNA sequence has an open reading frame encoding 500 amino acid residues, and the amino acid sequence derived from the open reading frame was identical with SEQ ID NO: 38. The derived protein also had a molecular weight of approximately 57 kDa.

The resultant full-length PIG8 cDNA clone was inserted into a multi-cloning site of the prokaryotic expression vector pBAD/thio-Topo (Invitrogen, U.S.) to obtain a vector pBAD/thio-Topo/PIG8, and Escherichia coli Top10 (Invitrogen, U.S.) was then transformed with the resultant pBAD/thio-Topo/PIG8. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate. FIG. 25 is a diagram showing an expression pattern of proteins of the E. coli Top10 strain transformed with the vector pBAD/thio-Topo/PIG8 using the SDS-PAGE, and it was revealed that a band of a fusion protein having a molecular weight of approximately 72 kDa was clearly observed after the L-arabinose induction. The 72-kDa fusion protein corresponds to a protein including the approximately 15-kDa HT-thioredoxin protein inserted into the vector pBAD/thio-Topo/PIG8, and the approximately 57-kDa PIG8 protein.

FIG. 25 is a diagram showing an SDS-PAGE analysis of the PIG8 protein. In FIG. 25, Lane 1 represents a protein sample before the L-arabinose induction, and Lane 2 represents a protein sample after expression of the PIG8 gene is induced by L-arabinose.

2-11: MRG1

The cDNA fragment MG21 obtained in Example 1-11 was labeled according to the method of the disclosure (Feinberg, A. P. and Vogelstein, B., Anal. Biochem., 132, 6-13 (1983)) to obtain a ³²P-labeled FC26 cDNA probe, and the ³²P-labeled FC26 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MRG1.

The full-length cDNA was sequenced, and therefore its DNA sequence was identical with SEQ ID NO: 41. The cDNA sequence has an open reading frame encoding 466 amino acid residues, and the amino acid sequence derived from the open reading frame was identical with SEQ ID NO: 42. The derived protein also had a molecular weight of approximately 52 kDa.

The resultant full-length MRG1 cDNA clone was inserted into a multi-cloning site of the prokaryotic expression vector pBAD/thio-Topo (Invitrogen, U.S.) to obtain a vector pBAD/thio-Topo/MRG1, and Escherichia coli Top10 (Invitrogen, U.S.) was then transformed with the resultant pBAD/thio-Topo/MRG1. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate. FIG. 26 is a diagram showing an expression pattern of proteins of the E. coli Top10 strain transformed with the vector pBAD/thio-Topo/MRG1 using the SDS-PAGE, and it was revealed that a band of a fusion protein having a molecular weight of approximately 67 kDa was clearly observed after the L-arabinose induction. The 67-kDa fusion protein corresponds to a protein including the approximately 15-kDa HT-thioredoxin protein inserted into the vector pBAD/thio-Topo/GIG1, and the approximately 52-kDa MRG11 protein.

FIG. 26 is a diagram showing an SDS-PAGE analysis of the MRG1 protein. In FIG. 26, Lane 1 represents a protein sample before the L-arabinose induction, and Lane 2 represents a protein sample after expression of the MRG1 gene is induced by L-arabinose.

2-12: PIG22

The cDNA fragment L989 obtained in Example 1-12 was labeled according to the method of the disclosure (Feinberg, A. P. and Vogelstein, B., Anal. Biochem., 132, 6-13 (1983)) to obtain a ³²P-labeled L989 cDNA probe, and the ³²P-labeled L989 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene PIG22.

The full-length cDNA was sequenced, and therefore its DNA sequence was identical with SEQ ID NO: 45. The cDNA sequence has an open reading frame encoding 350 amino acid residues, and the amino acid sequence derived from the open reading frame was identical with SEQ ID NO: 46. The derived protein also had a molecular weight of approximately 40 kDa.

The transformed E. coli strain was incubated in LB broth, and then 1 mM isopropy-1-β-D-thiogalactopyranoside (IPTG) was added to the culture, and reacted at 37° C. for 3 hours to express the PIG22 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

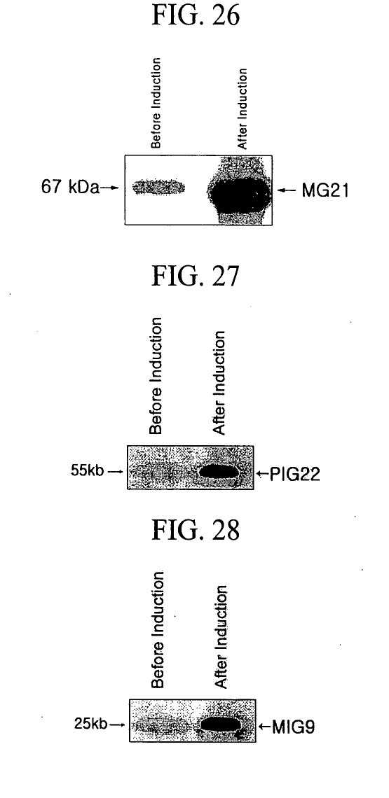

FIG. 27 is a diagram showing an SDS-PAGE analysis of the PIG22 protein. In FIG. 27, Lane 1 represents a protein sample before the IPTG induction, and Lane 2 represents a protein sample after expression of the PIG22 gene is induced by IPTG. As shown in FIG. 27, the expressed PIG22 protein has a molecular weight of approximately 40 kDa, which corresponds to a molecular weight of a protein derived from its DNA sequence.

2-13: MIG9

The cDNA fragment L741 obtained in Example 1-13 was labeled according to the method of the disclosure (Feinberg, A. P. and Vogelstein, B., Anal. Biochem., 132, 6-13 (1983)) to obtain a ³²P-labeled L935 cDNA probe, and the ³²P-labeled L935 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MIG9.

The full-length cDNA was sequenced, and therefore its DNA sequence was identical with SEQ ID NO: 49. The cDNA sequence has an open reading frame encoding 88 amino acid residues, and the amino acid sequence derived from the open reading frame was identical with SEQ ID NO: 50. The derived protein also had a molecular weight of approximately 10 kDa.

The transformed E. coli strain was incubated in LB broth, and then 1 mM isopropy-1-β-D-thiogalactopyranoside (IPTG) was added to the culture, and reacted at 37° C. for 3 hours to express the MIG9 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

FIG. 28 is a diagram showing an SDS-PAGE analysis of the MIG9 protein. In FIG. 28, Lane 1 represents a protein sample before the IPTG induction, and Lane 2 represents a protein sample after expression of the MIG9 gene is induced by IPTG. As shown in FIG. 28, the expressed MIG9 protein has a molecular weight of approximately 10 kDa, which corresponds to a molecular weight of a protein derived from its DNA sequence.

2-14: MIG11

The cDNA fragment L861 obtained in Example 1-14 was labeled according to the method of the disclosure (Feinberg, A. P. and Vogelstein, B., Anal. Biochem., 132, 6-13 (1983)) to obtain a ³²P-labeled L935 cDNA probe, and the ³²P-labeled L935 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MIG11.

The full-length cDNA was sequenced, and therefore its DNA sequence was identical with SEQ ID NO: 53. The cDNA sequence has an open reading frame encoding 249 amino acid residues, and the amino acid sequence derived from the open reading frame was identical with SEQ ID NO: 54. The derived protein also had a molecular weight of approximately 27 kDa.

The transformed E. coli strain was incubated in LB broth, and then 1 mM isopropy-1-β-D-thiogalactopyranoside (IPTG) was added to the culture, and reacted at 37° C. for 3 hours to express the MIG11 gene. A protein sample was obtained from the culture, and then SDS-PAGE was conducted with the protein sample according to the method as described in the disclosure (Sambrook, J. et al., Molecular Cloning: A Laboratory mannual, New York: Cold Spring Harbor Laboratory (1989)).

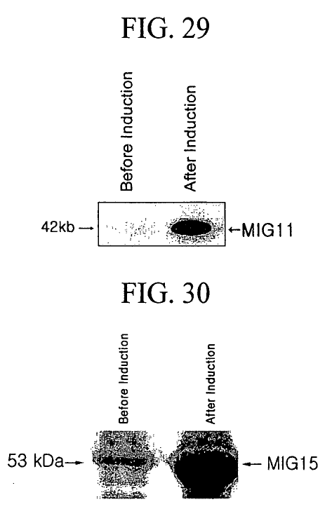

FIG. 29 is a diagram showing an SDS-PAGE analysis of the MIG11 protein. In FIG. 29, Lane 1 represents a protein sample before the IPTG induction, and Lane 2 represents a protein sample after expression of the MIG11 gene is induced by IPTG. As shown in FIG. 29, the expressed MIG11 protein has a molecular weight of approximately 27 kDa, which corresponds to a molecular weight of a protein derived from its DNA sequence.

2-15: MIG15

The cDNA fragment CC312 obtained in Example 1-15 was labeled according to the method of the disclosure (Feinberg, A. P. and Vogelstein, B., Anal. Biochem., 132, 6-13 (1983)) to obtain a ³²P-labeled CC312 cDNA probe, and the ³²P-labeled CC312 cDNA probe was plaque-hybridized with bacteriophage λgt11 human lung embryonic fibroblast cDNA library (Miki, T. et al., Gene, 83, 137-146 (1989)) according to the method as described in the disclosure (Sambrook, J. et al., Molecular Cloning: A Laboratory mannual, New York: Cold Spring Harbor Laboratory (1989)) to obtain a full-length cDNA clone of the human cancer suppressor gene MIG15.

The full-length cDNA was sequenced, and therefore its DNA sequence was identical with SEQ ID NO: 57. The cDNA sequence has an open reading frame encoding 323 amino acid residues, and the amino acid sequence derived from the open reading frame was identical with SEQ ID NO: 58. The derived protein also had a molecular weight of approximately 38 kDa.

The resultant full-length MIG15 cDNA clone was inserted into a multi-cloning site of the prokaryotic expression vector pBAD/thio-Topo (Invitrogen, U.S.) to obtain a vector pBAD/thio-Topo/MIG15, and Escherichia coli Top10 (Invitrogen, U.S.) was then transformed with the resultant pBAD/thio-Topo/MIG15. The proteins HT-Thioredoxin to be expressed was inserted upstream of the multi-cloning site of the vector pBAD/thio-Topo. The transformed E. coli strain was incubated in LB broth with shaking, and the resultant culture was diluted 1/100, and then reacted for 3 hours again. 0.5 mM L-arabinose (Sigma, U.S.) was added thereto to induce production of proteins. The E. coli cell in the culture medium was sonicated before and after the L-arabinose induction, and then 12% sodium dodecyl sulphate polyacrylamide gel electrophoresis (SDS-PAGE) was conducted with the sonicated homogenate. FIG. 2 is a diagram showing an expression pattern of proteins of the E. coli Top10 strain transformed with the vector pBAD/thio-Topo/MIG15 using the SDS-PAGE, and it was revealed that a band of a fusion protein having a molecular weight of approximately 53 kDa was clearly observed after the L-arabinose induction. The 53-kDa fusion protein corresponds to a protein including the approximately 15-kDa HT-thioredoxin protein inserted into the vector pBAD/thio-Topo/MIG15, and the approximately 38-kDa MIG15 protein.

FIG. 30 is a diagram showing an SDS-PAGE analysis of the MIG15 protein. In FIG. 30, Lane 1 represents a protein sample before the IPTG induction, and Lane 2 represents a protein sample after expression of the MIG15 gene is induced by IPTG.

### Example 3

### Northern Blotting of Gene

3-1. GIG1

In order to assess an expression level of the GIG1 gene, the northern blotting was carried out, as follows.

20 μg of each of the total RNA samples obtained from the three normal exocervical tissues, the three primary cervical cancer tissues and the two cervical cancer cell lines as obtained in Example 1-1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length GIG1 cDNA obtained in Example 1-1. The northern blotting procedure was repeated twice; one was quantitified using the densitometer and the other was hybridized with the β-actin probe to determine the total mRNA.

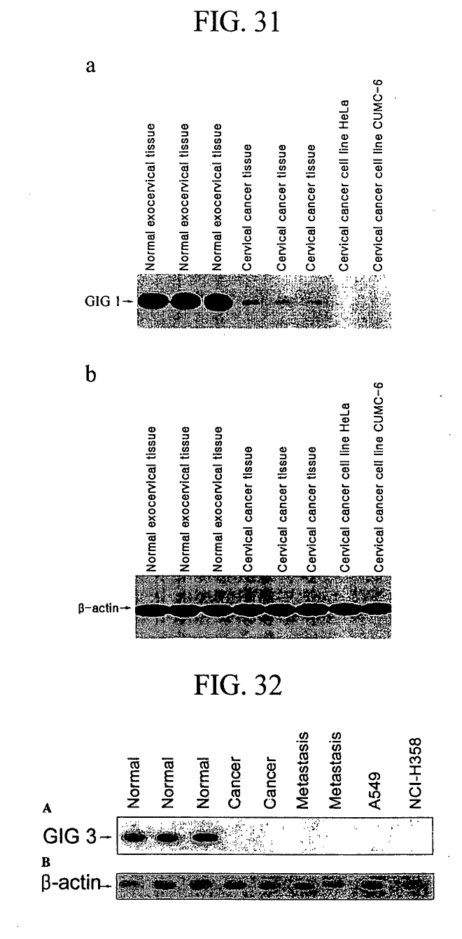

FIG. 31(a) shows the northern blotting result that the GIG1 gene is differentially expressed in a normal exocervical tissue, a primary cervical cancer tissue and a cervical cancer cell line, and FIG. 31(b) is a northern blotting result showing expression of β-actin. In FIGS. 31(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 31(a) and (b), it was revealed that the expression level of the GIG1 gene was highly detected all in the three samples of the normal exocervical tissue, but its expression level was significantly lower in the three samples of the cervical cancer tissue than the normal tissue, and not detected in the two samples of the cervical cancer cell line.

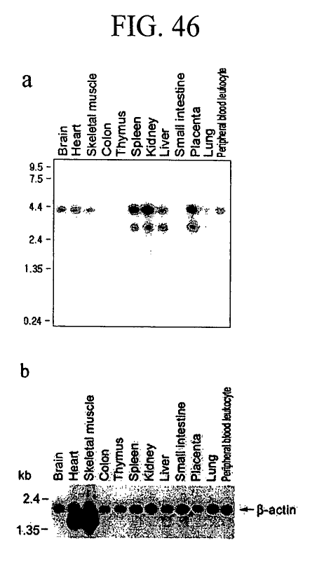

FIG. 46(a) shows a northern blotting result that the GIG1 gene is differentially expressed in various normal tissues, and FIG. 46(b) shows a northern blotting result obtained by hybridizing the same blot with β-actin probe. As shown in FIG. 46(a), a dominant GIG1 mRNA transcript having a size of approximately 4.0 kb was overexpressed and a transcript having a size of approximately 3.0 kb was additionally expressed in the normal tissues such as brain, heart, skeletal muscles, spleen, kidney, liver, placenta, lungs and peripheral leukocyte.

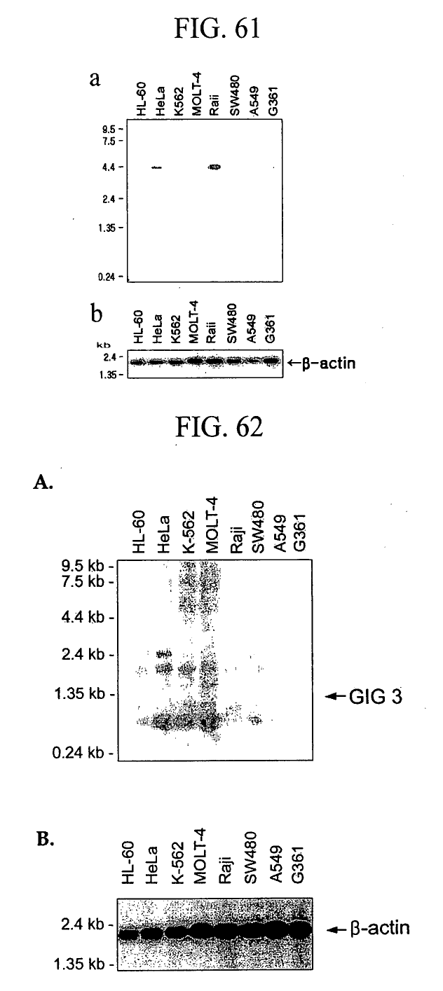

FIG. 61(a) shows a northern blotting result that the GIG1 gene is differentially expressed in various cancer cell lines, and FIG. 61(b) shows a northern blotting result obtained by hybridizing the same blot with β-actin probe. As shown in FIG. 61(a), the GIG1 gene was slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the GIG1 gene of the present invention had the tumor suppresser function in the normal tissues such as cervix, brain, heart, skeletal muscles, spleen, kidney, liver, placenta, lungs and peripheral leukocyte.

3-2: GIG3

In order to assess an expression level of the GIG3 gene, the northern blotting was carried out, as follows.

20 μg of each of the total RNA samples obtained from the three normal lung tissues, the two primary lung cancer tissues, the two metastatic lung cancer tissues and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG3 cDNA using the Rediprime II random prime labelling system (Amersham, United Kingdom). The northern blotting procedure was repeated twice; one was quantitified using the densitometer and the other was hybridized with the β-actin probe to determine the total mRNA.

FIG. 32(a) shows the northern blotting result that the GIG3 gene is differentially expressed in a normal lung tissue, a primary lung cancer tissue, a metastatic lung cancer tissue and a lung cancer cell line, and FIG. 32(b) shows the northern blotting result obtained by hybridizing the same blot with β-actin probe. As shown in FIGS. 32(a) and (b), it was revealed that the expression level of the GIG3 gene was highly detected all in the three samples of the normal lung tissue, but its expression level was not detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

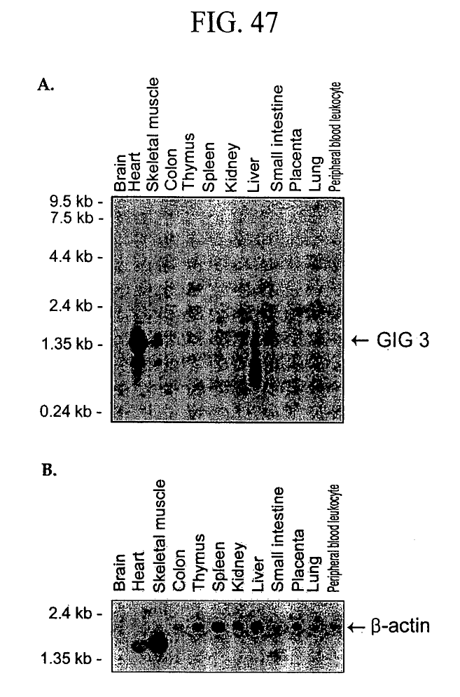

FIG. 47(a) shows a northern blotting result that the GIG3 gene is differentially expressed in various normal tissues, and FIG. 47(b) shows a northern blotting result obtained by hybridizing the same blot with β-actin probe. As shown in FIG. 47(a), a dominant GIG2 mRNA transcript having a size of approximately 1.3 kb was highly overexpressed in the normal tissues such as lungs, heart, muscles, kidney and liver. In addition, a transcript having a size of approximately 0.7 kb was also expressed in the normal tissues.

FIG. 62(a) shows a northern blotting result that the GIG3 gene is differentially expressed in various cancer cell lines, and FIG. 62(b) shows a northern blotting result obtained by hybridizing the same blot with β-actin probe. As shown in FIG. 62(a), the approximately 1.3-kb dominant GIG3 mRNA transcript detected in the normal tissues was not at all expressed but transcripts having different sizes of approximately 2.0 or 0.5 kb was slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the GIG3 gene of the present invention had the tumor suppresser function in the normal tissues such as lungs, heart, muscles, kidney and liver.

3-3: GIG4

In order to assess an expression level of the GIG4 gene, the northern blotting was carried out, as follows.

20 μg of each of the total RNA samples obtained from the three normal lung tissues, the two primary lung cancer tissues, the two metastatic lung cancer tissues and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG4 cDNA using the Rediprime II random prime labelling system (Amersham, United Kingdom). The northern blotting procedure was repeated twice; one was quantitified using the densitometer and the other was hybridized with the β-actin probe to determine the total mRNA.

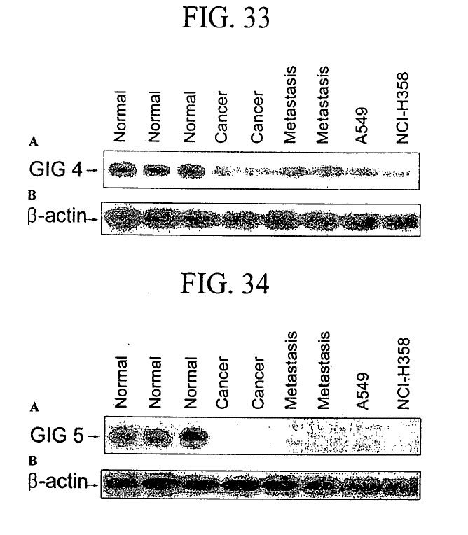

FIG. 33(a) shows the northern blotting result that the GIG4 gene is differentially expressed in a normal lung tissue, a primary lung cancer tissue, a metastatic lung cancer tissue and a lung cancer cell line, and FIG. 33(b) shows the northern blotting result obtained by hybridizing the same blot with β-actin probe. As shown in FIGS. 33(a) and (b), it was revealed that the expression level of the GIG4 gene was highly detected all in the three samples of the normal lung tissue, but its expression level was slightly detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

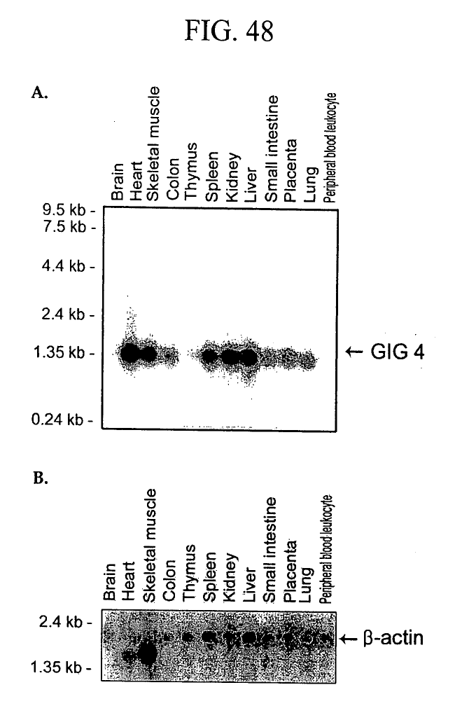

FIG. 48(a) shows a northern blotting result that the GIG4 gene is differentially expressed in various normal tissues, and FIG. 48(b) shows a northern blotting result obtained by hybridizing the same blot with β-actin probe. As shown in FIG. 48(a), a dominant GIG4 mRNA transcript having a size of approximately 1.3 kb was highly overexpressed in the normal tissues such as heart, muscles, spleen, kidney and liver. In addition, a transcript having a size of approximately 0.7 kb was also expressed in the normal tissues in the normal tissues such as large and small intestines and placenta.

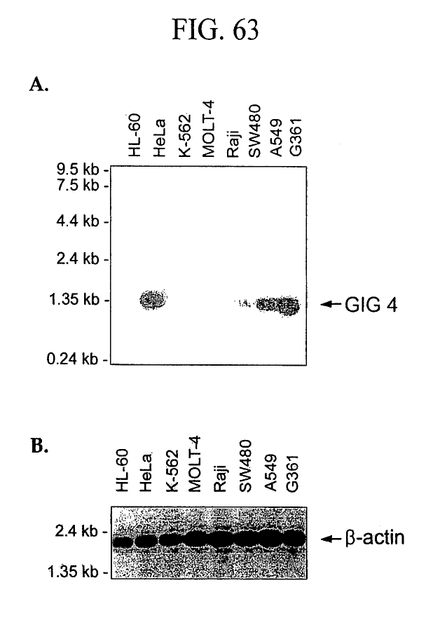

FIG. 63(a) shows a northern blotting result that the GIG4 gene is differentially expressed in various cancer cell lines, and FIG. 63(b) shows a northern blotting result obtained by hybridizing the same blot with β-actin probe. As shown in FIG. 63(a), the approximately 1.3-kb dominant GIG4 mRNA transcript detected in the normal tissues was slightly expressed in the tissues such as HeLa cervical cancer cell, A549 lung cancer cell and G361 melanoma cell but not at all expressed in the tissues such as promyelocytic leukemia HL-60, a chronic myelocytic leukemia cell line K-562, lymphoblastoid leukemia MOLT-4, Burkitt's lymphoma (Raji) and SW480 colon cancer cell. From such a result, it was revealed that the GIG4 gene of the present invention had the tumor suppresser function in the normal tissues such as lungs, heart, muscles, spleen, kidney, liver, large and small intestines and placenta.

3-4: GIG5

In order to assess an expression level of the GIG5 gene, the northern blotting was carried out, as follows.

20 μg of each of the total RNA samples obtained from the three normal lung tissues, the two primary lung cancer tissues, the two metastatic lung cancer tissues and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GIG5 cDNA using the Rediprime II random prime labelling system (Amersham, United Kingdom). The northern blotting procedure was repeated twice; one was quantitified using the densitometer and the other was hybridized with the β-actin probe to determine the total mRNA.

FIG. 34(a) shows the northern blotting result that the GIG5 gene is differentially expressed in a normal lung tissue, a primary lung cancer tissue, a metastatic lung cancer tissue and a lung cancer cell line, and FIG. 34(b) shows the northern blotting result obtained by hybridizing the same blot with β-actin probe. As shown in FIGS. 34(a) and (b), it was revealed that the expression level of the GIG5 gene was highly detected all in the three samples of the normal lung tissue, but its expression level was not detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

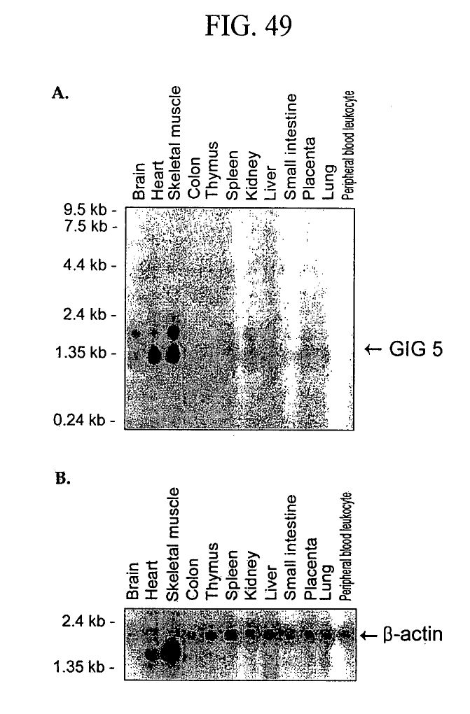

FIG. 49(a) shows a northern blotting result that the GIG5 gene is differentially expressed in various normal tissues, and FIG. 49(b) shows a northern blotting result obtained by hybridizing the same blot with β-actin probe. As shown in FIG. 49(a), a dominant GIG2 mRNA transcript having a size of approximately 1.2 kb was highly overexpressed and a transcript having a size of approximately 2.0 kb was additionally highly expressed in the normal tissues such as heart and muscle. Also, the 1.2-kb and 2.0-kb mRNA transcripts were sightly expressed in the normal tissues such as brain, colon, thymus, spleen, kidney, liver, small intestines and placenta.

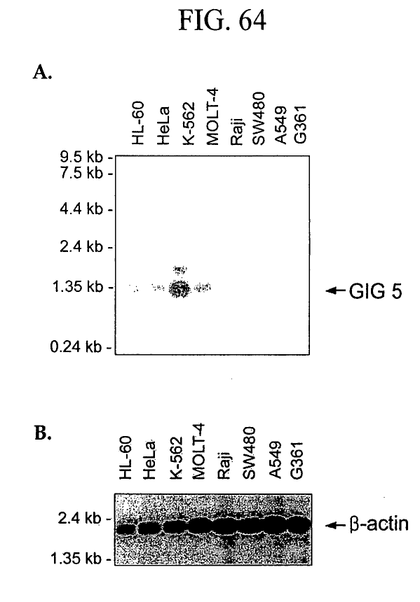

FIG. 64(a) shows a northern blotting result that the GIG5 gene is differentially expressed in various cancer cell lines, and FIG. 64(b) shows a northern blotting result obtained by hybridizing the same blot with β-actin probe. As shown in FIG. 64(a), the approximately 1.3-kb dominant GIG5 mRNA transcript and the approximately 2.0-kb transcript detected in the normal tissues was very slightly expressed or not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the GIG5 gene of the present invention had the tumor suppresser function in the normal tissues such as lungs, hear, and muscles.

3-5: GIG11

In order to assess an expression level of the GIG11 gene, the northern blotting was carried out, as follows.

20 μg of each of the total RNA samples obtained from the three normal breast tissues, the three primary breast cancer tissues and the breast cancer cell line MCF-7 as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the partial sequence BBCC311N cDNA of the full-length GIG11 cDNA using the Rediprime II random prime labelling system (Amersham, United Kingdom). The northern blotting procedure was repeated twice; one was quantitified using the densitometer and the other was hybridized with the β-actin probe to determine the total mRNA.

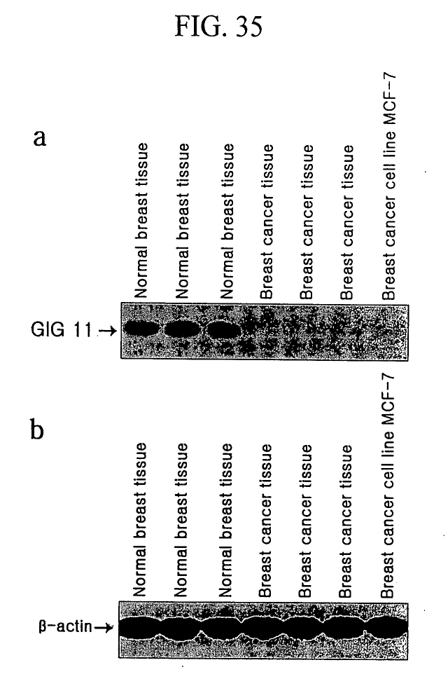

FIG. 35(a) shows the northern blotting result that the GIG11 gene is differentially expressed in a normal breast tissue, a primary breast cancer tissue and a breast cancer cell line, and FIG. 35(b) shows the northern blotting result obtained by hybridizing the same blot with β-actin probe. As shown in FIGS. 35(a) and (b), it was revealed that the expression level of the GIG11 gene was highly detected all in the three samples of the normal breast tissue, but its expression level was significantly lower in the two samples of the breast cancer tissue than the normal tissue, and very slightly detected even in the one sample of the breast cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

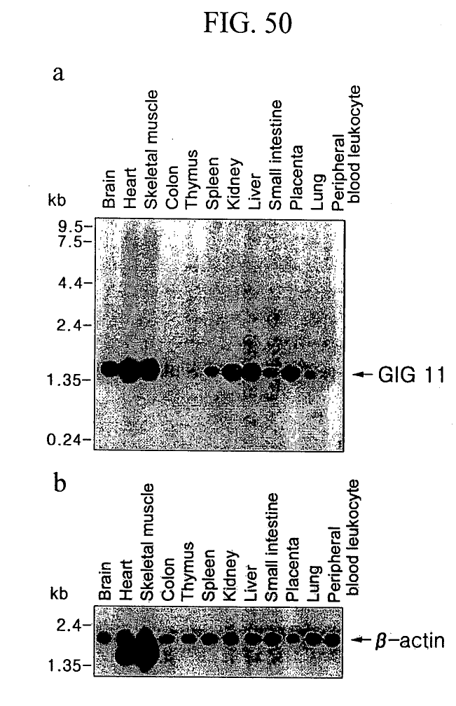

FIG. 50(a) shows a northern blotting result that the GIG11 gene is differentially expressed in various normal tissues, and FIG. 50(b) shows a northern blotting result obtained by hybridizing the same blot with β-actin probe. As shown in FIG. 50(a), a dominant GIG11 mRNA transcript having a size of approximately 1.5 kb was overexpressed in the normal tissues such as breast, brain, heart, muscles, thymus, spleen, kidney, liver, small intestines, placenta and lungs. In addition, the 1.5-kb GIG11 mRNA transcript was expressed even in the normal tissues such as large intestines and peripheral blood.

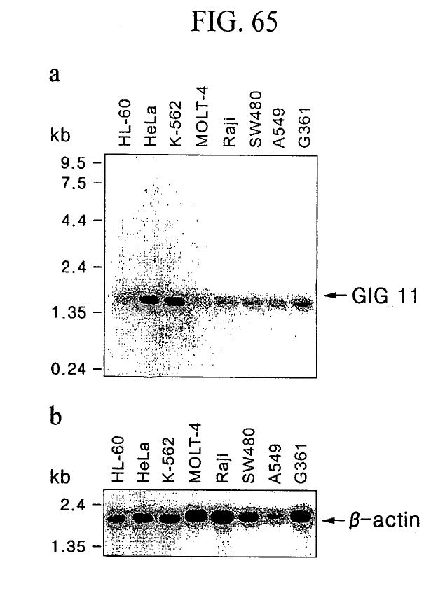

FIG. 65(a) shows a northern blotting result that the GIG11 gene is differentially expressed in various cancer cell lines, and FIG. 65(b) shows a northern blotting result obtained by hybridizing the same blot with β-actin probe. As shown in FIG. 65(a), the GIG11 gene was very slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the GIG11 gene of the present invention had the tumor suppresser function in the normal tissues such as breast, brain, heart, muscles, thymus, spleen, kidney, liver, small intestines, placenta, lungs, large intestines and peripheral blood.

3-6: MIG2

In order to assess an expression level of the MIG2 gene, the northern blotting was carried out, as follows.

20 μg of each of the total RNA samples obtained from the three normal exocervical tissues, the three primary cervical cancer tissues and the two cervical cancer cell line as obtained in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length MIG2 cDNA obtained in Example 1. The northern blotting procedure was repeated twice; one was quantitified using the densitometer and the other was hybridized with the β-actin probe to determine the total mRNA.

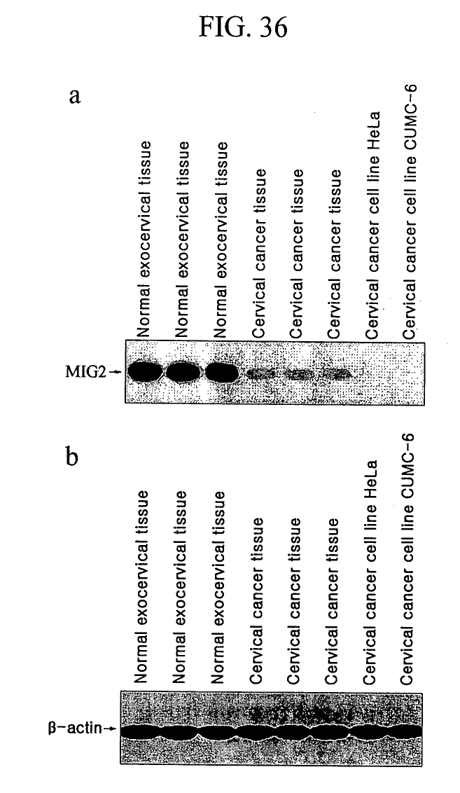

FIG. 36(a) shows the northern blotting result that the MIG2 gene is differentially expressed in a normal exocervical tissue, a primary cervical cancer tissue and a cervical cancer cell line, and FIG. 36(b) is a northern blotting result showing expression of β-actin. In FIGS. 36(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 36(a) and (b), it was revealed that the expression level of the MIG2 gene was highly detected all in the three samples of the normal exocervical tissue, but its expression level was significantly lower in the three samples of the cervical cancer tissue than the normal tissue, and not detected in the two samples of the cervical cancer cell line.

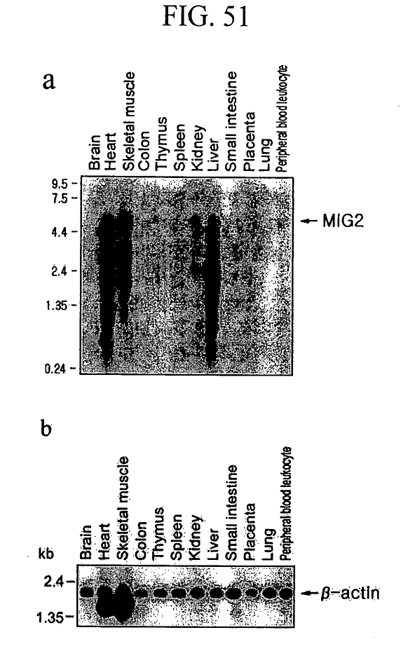

FIG. 51(a) shows a northern blotting result that the MIG2 gene is differentially expressed in various normal tissues, and FIG. 51(b) shows a northern blotting result obtained by hybridizing the same blot with β-actin probe. As shown in FIG. 51(a), a dominant MIG2 mRNA transcript having a size of approximately 5.0 kb was overexpressed and a transcript having a size of approximately 2.0 kb was also expressed in the normal tissues such as heart, skeletal muscles, kidney and liver.

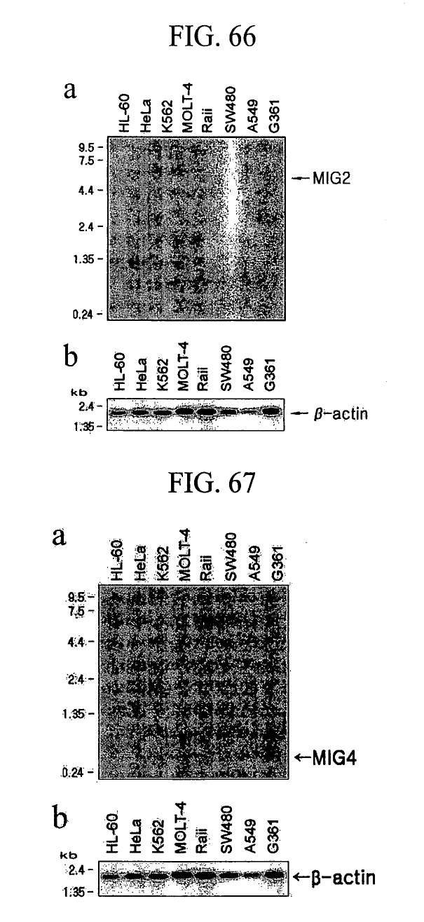

FIG. 66 shows a northern blotting result that the MIG2 gene is differentially expressed in various cancer cell lines, and FIG. 66(b) shows a northern blotting result obtained by hybridizing the same blot with β-actin probe. As shown in FIG. 66(a), the MIG2 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the MIG2 gene of the present invention had the tumor suppresser function in the normal tissues such as cervix, heart, skeletal muscles, kidney, liver, lungs and peripheral leukocyte.

3-7: MIG4

In order to assess an expression level of the MIG4 gene, the northern blotting was carried out, as follows.

20 μg of each of the total RNA samples obtained from the three normal exocervical tissues, the three primary cervical cancer tissues and the two cervical cancer cell line as obtained in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length MIG4 cDNA obtained in Example 1. The northern blotting procedure was repeated twice; one was quantitified using the densitometer and the other was hybridized with the β-actin probe to determine the total mRNA.

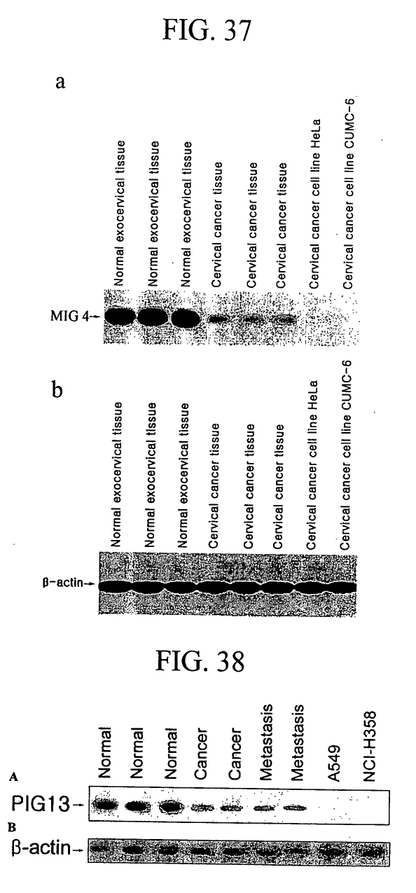

FIG. 37(a) shows the northern blotting result that the MIG4 gene is differentially expressed in a normal exocervical tissue, a primary cervical cancer tissue and a cervical cancer cell line, and FIG. 37(b) is a northern blotting result showing expression of β-actin. In FIGS. 37(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 37(a) and (b), it was revealed that the expression level of the MIG4 gene was highly detected all in the three samples of the normal exocervical tissue, but its expression level was significantly lower in the three samples of the cervical cancer tissue than the normal tissue, and not detected in the two samples of the cervical cancer cell line.

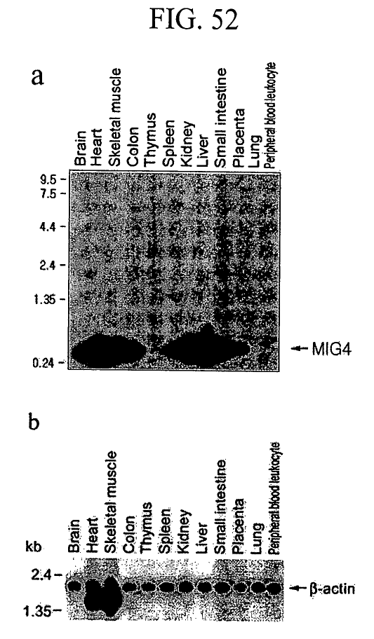

FIG. 52(a) shows a northern blotting result that the MIG4 gene is differentially expressed in various normal tissues, and FIG. 52(b) shows a northern blotting result obtained by hybridizing the same blot with β-actin probe. As shown in FIG. 52(a), a dominant MIG4 mRNA transcript having a size of approximately 0.5 kb was overexpressed and a transcript having a size of approximately 2.0 kb was also expressed in the normal tissues such as brain, heart, skeletal muscles, large intestines, spleen, kidney, liver, placenta, lungs and peripheral blood leukocyte.

FIG. 67 shows a northern blotting result that the MIG4 gene is differentially expressed in various cancer cell lines, and FIG. 67(b) shows a northern blotting result obtained by hybridizing the same blot with β-actin probe. As shown in FIG. 67(a), the MIG4 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the MIG4 gene of the present invention had the tumor suppresser function in the normal tissues such as cervix, brain, heart, skeletal muscles, large intestines, spleen, kidney, liver, placenta, lungs and peripheral blood leukocyte.

3-8: PIG13

In order to assess an expression level of the PIG13 gene, the northern blotting was carried out, as follows.

20 μg of each of the total RNA samples obtained from the three normal lung tissues, the two primary lung cancer tissues, the two metastatic lung cancer tissue and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length PIG13 cDNA using the Rediprime II random prime labelling system (Amersham, United Kingdom). The northern blotting procedure was repeated twice; one was quantitified using the densitometer and the other was hybridized with the β-actin probe to determine the total mRNA.

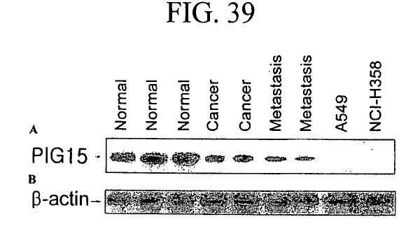

FIG. 38(a) shows the northern blotting result that the PIG13 gene is differentially expressed in a normal lung tissue, a primary lung cancer tissue, a metastatic lung cancer tissue and a lung cancer cell line, and FIG. 38(b) shows the northern blotting result obtained by hybridizing the same blot with β-actin probe. As shown in FIGS. 38(a) and (b), it was revealed that the expression level of the PIG13 gene was highly detected all in the three samples of the normal lung tissue, but its expression level was very slightly expressed or not detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

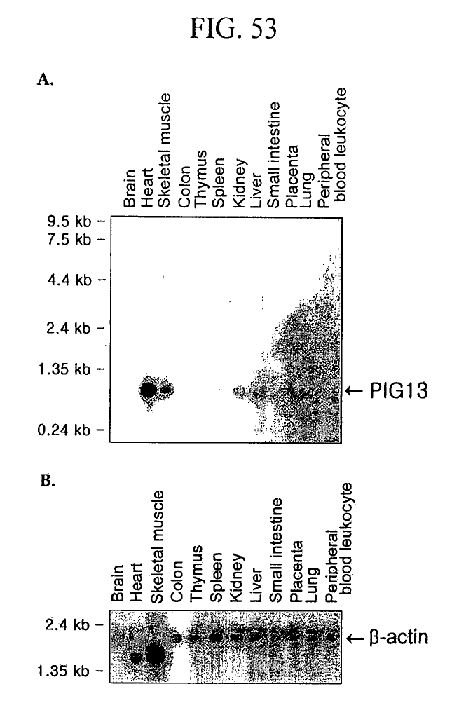

FIG. 53(a) shows a northern blotting result that the PIG13 gene is differentially expressed in various normal tissues, and FIG. 53(b) shows a northern blotting result obtained by hybridizing the same blot with β-actin probe. As shown in FIG. 53(a), a dominant PIG13 mRNA transcript having a size of approximately 1.0 kb was highly overexpressed in the normal tissues such as lungs, brain, heart, muscles, large intestines, spleen, kidney, liver and small intestines. In addition, a transcript having a size of approximately 4.5 kb was also expressed in the normal tissues.

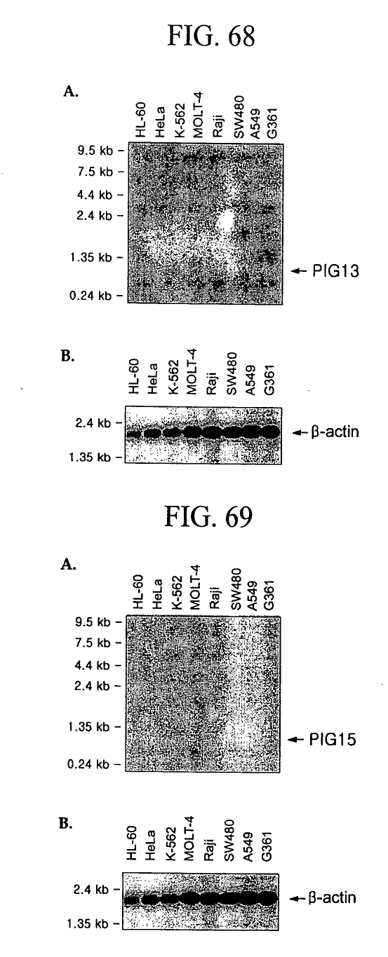

FIG. 68(a) shows a northern blotting result that the PIG13 gene is differentially expressed in various cancer cell lines, and FIG. 68(b) shows a northern blotting result obtained by hybridizing the same blot with β-actin probe. As shown in FIG. 68(a), the approximately 1.0-kb dominant PIG13 mRNA transcript detected in the normal tissues was not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the PIG13 gene of the present invention had the tumor suppresser function in the normal tissues such as lungs, brain, heart, muscles, large intestines, spleen, kidney, liver, and small intestines.

3-9: PIG15

In order to assess an expression level of the PIG15 gene, the northern blotting was carried out, as follows.

20 μg of each of the total RNA samples obtained from the three normal lung tissues, the two primary lung cancer tissues, the two metastatic lung cancer tissue and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length PIG15 cDNA using the Rediprime II random prime labelling system (Amersham, United Kingdom). The northern blotting procedure was repeated twice; one was quantitified using the densitometer and the other was hybridized with the β-actin probe to determine the total mRNA.

FIG. 39(a) shows the northern blotting result that the PIG15 gene is differentially expressed in a normal lung tissue, a primary lung cancer tissue, a metastatic lung cancer tissue and a lung cancer cell line, and FIG. 39(b) shows the northern blotting result obtained by hybridizing the same blot with β-actin probe. As shown in FIGS. 39(a) and (b), it was revealed that the expression level of the PIG15 gene was highly detected all in the three samples of the normal lung tissue, but its expression level was very slightly expressed or not detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

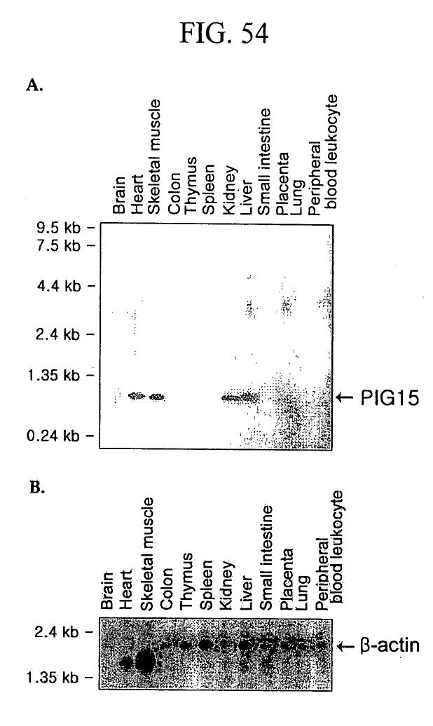

FIG. 54(a) shows a northern blotting result that the PIG15 gene is differentially expressed in various normal tissues, and FIG. 54(b) shows a northern blotting result obtained by hybridizing the same blot with β-actin probe. As shown in FIG. 54(a), a dominant PIG15 mRNA transcript having a size of approximately 1.0 kb was highly overexpressed in the normal tissues such as lungs, brain, heart, muscles, large intestines, spleen, kidney, liver, and small intestines.

FIG. 69(a) shows a northern blotting result that the PIG15 gene is differentially expressed in various cancer cell lines, and FIG. 69(b) shows a northern blotting result obtained by hybridizing the same blot with β-actin probe. As shown in FIG. 69(a), the approximately 1.0-kb dominant PIG15 mRNA transcript detected in the normal tissues was not at all expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the PIG15 gene of the present invention had the tumor suppresser function in the normal tissues such as lungs, brain, heart, muscles, large intestines, spleen, kidney, liver, and small intestines.

3-10: PIG8

In order to assess an expression level of the PIG8 gene, the northern blotting was carried out, as follows.

20 μg of each of the total RNA samples obtained from the three normal exocervical tissues, the three primary cervical cancer tissues and the two cervical cancer cell line as obtained in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length PIG8 cDNA obtained in Example 1-1. The northern blotting procedure was repeated twice; one was quantitified using the densitometer and the other was hybridized with the β-actin probe to determine the total mRNA.

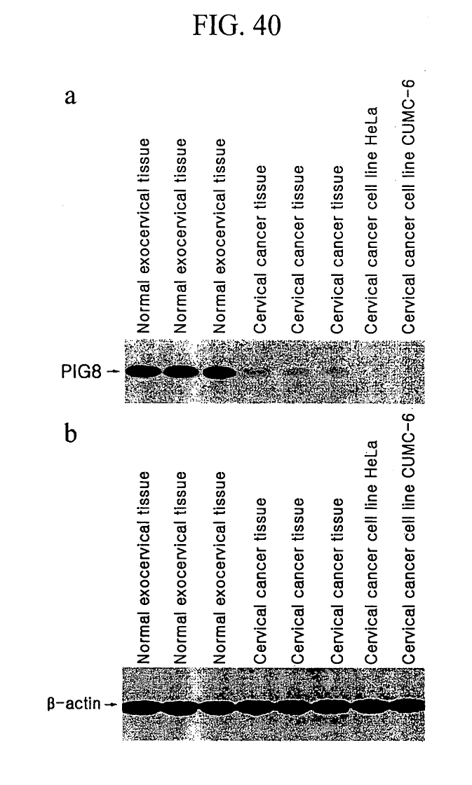

FIG. 40(a) shows the northern blotting result that the PIG8 gene is differentially expressed in a normal exocervical tissue, a primary cervical cancer tissue and a cervical cancer cell line, and FIG. 40(b) is a northern blotting result showing expression of β-actin. In FIGS. 40(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 40(a) and (b), it was revealed that the expression level of the PIG8 gene was highly detected all in the three samples of the normal exocervical tissue, but its expression level was significantly lower in the three samples of the cervical cancer tissue than the normal tissue, and not detected in the two samples of the cervical cancer cell line.

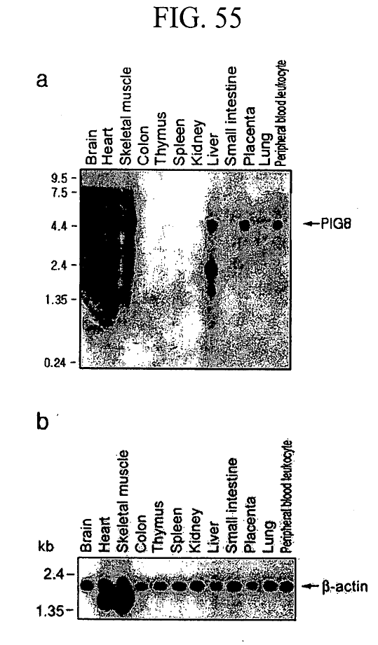

FIG. 55(a) shows a northern blotting result that the PIG8 gene is differentially expressed in various normal tissues, and FIG. 55(b) shows a northern blotting result obtained by hybridizing the same blot with β-actin probe. As shown in FIG. 55(a), a dominant PIG8 mRNA transcript having a size of approximately 4.5 kb was overexpressed in the normal tissues such as brain, heart, skeletal muscles, liver, placenta and peripheral leukocyte, and a transcript having a size of approximately 2.2 kb was also expressed in the normal liver tissue.

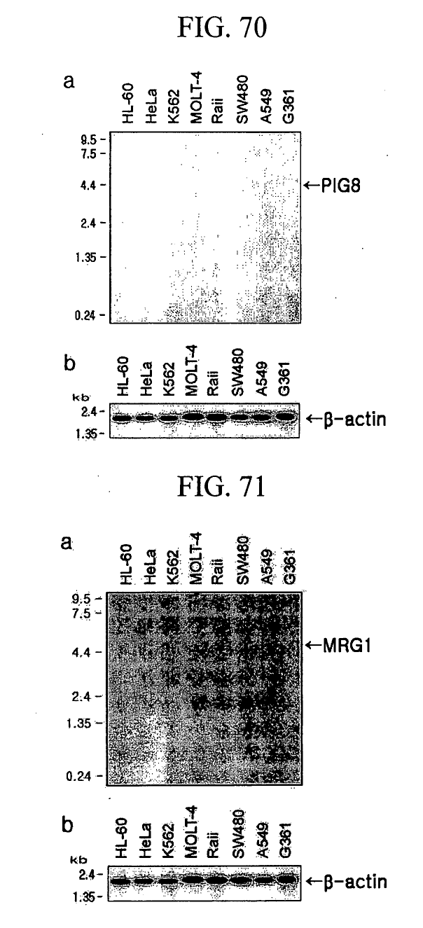

FIG. 70(a) shows a northern blotting result that the PIG8 gene is differentially expressed in various cancer cell lines, and FIG. 70(b) shows a northern blotting result obtained by hybridizing the same blot with β-actin probe. As shown in FIG. 70(a), the PIG8 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the PIG8 gene of the present invention had the tumor suppresser function in the normal tissues such as cervix, brain, heart, skeletal muscles, liver, placenta and peripheral leukocyte.

3-11: MRG1

In order to assess an expression level of the MRG1 gene, the northern blotting was carried out, as follows.

20 μg of each of the total RNA samples obtained from the three normal exocervical tissues, the three primary cervical cancer tissues and the two cervical cancer cell line as obtained in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length MRG1 cDNA obtained in Example 1-1. The northern blotting procedure was repeated twice; one was quantitified using the densitometer and the other was hybridized with the β-actin probe to determine the total mRNA.

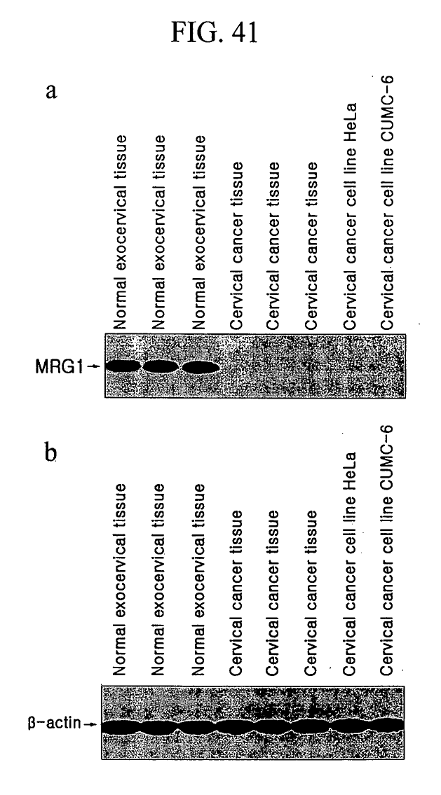

FIG. 41(a) shows the northern blotting result that the MRG1 gene is differentially expressed in a normal exocervical tissue, a primary cervical cancer tissue and a cervical cancer cell line, and FIG. 41(b) is a northern blotting result showing expression of β-actin. In FIGS. 41(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 41(a) and (b), it was revealed that the expression level of the MRG1 gene was highly detected all in the three samples of the normal exocervical tissue, but its expression level was not detected in the three samples of the cervical cancer tissue, and also not detected in the two samples of the cervical cancer cell line.

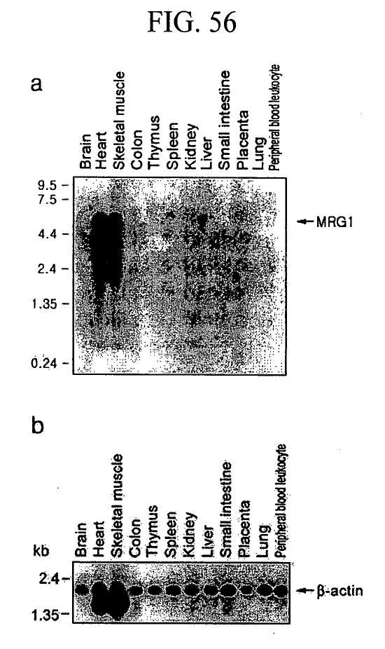

FIG. 56(a) shows a northern blotting result that the MRG1 gene is differentially expressed in various normal tissues, and FIG. 56(b) shows a northern blotting result obtained by hybridizing the same blot with β-actin probe. As shown in FIG. 56(a), a dominant MRG1 mRNA transcript having a size of approximately 5.0 kb was overexpressed and a transcript having a size of approximately 2.0 kb was also expressed in the normal tissues such as heart, skeletal muscles, kidney, liver and placenta.

FIG. 71(a) shows a northern blotting result that the MRG1 gene is differentially expressed in various cancer cell lines, and FIG. 71(b) shows a northern blotting result obtained by hybridizing the same blot with β-actin probe. As shown in FIG. 71(a), the MRG1 gene was not expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the MRG1 gene of the present invention had the tumor suppresser function in the normal tissues such as cervix, heart, skeletal muscles, kidney, liver, placenta, lungs and peripheral leukocyte.

3-12: PIG22

In order to assess an expression level of the PIG22 gene, the northern blotting was carried out, as follows.

20 μg of each of the total RNA samples obtained from the three normal lung tissues, the two primary lung cancer tissues, the two metastatic lung cancer tissue and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length GPIG22 cDNA using the Rediprime II random prime labelling system (Amersham, United Kingdom). The northern blotting procedure was repeated twice; one was quantitified using the densitometer and the other was hybridized with the β-actin probe to determine the total mRNA.

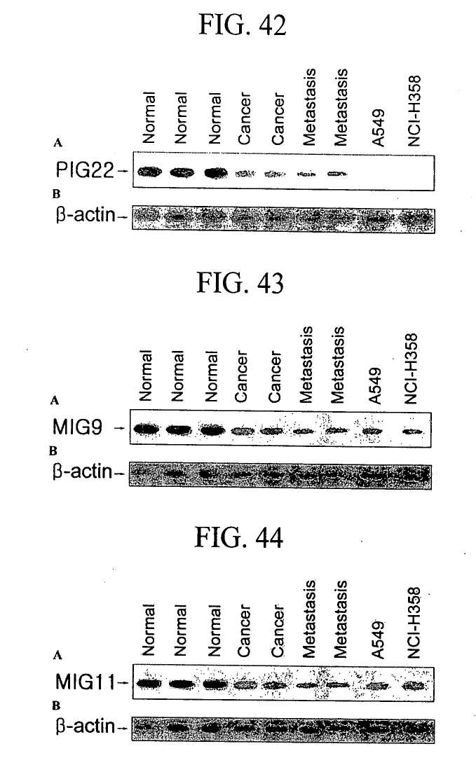

FIG. 42(a) shows the northern blotting result that the PIG22 gene is differentially expressed in a normal lung tissue, a primary lung cancer tissue, a metastatic lung cancer tissue and a lung cancer cell line, and FIG. 42(b) shows the northern blotting result obtained by hybridizing the same blot with β-actin probe. As shown in FIGS. 42(a) and (b), it was revealed that the expression level of the PIG22 gene was highly detected all in the three samples of the normal lung tissue, but its expression level was slightly detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

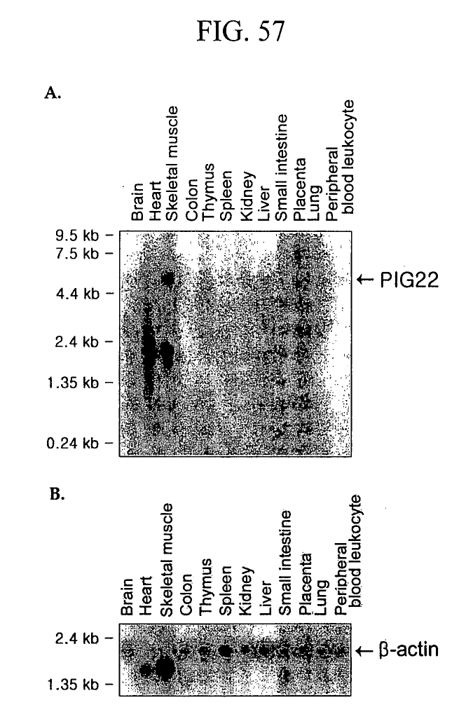

FIG. 57(a) shows a northern blotting result that the PIG22 gene is differentially expressed in various normal tissues, and FIG. 57(b) shows a northern blotting result obtained by hybridizing the same blot with β-actin probe. As shown in FIG. 57(a), a dominant PIG22 mRNA transcript having a size of approximately 5 kb was highly overexpressed in the normal tissues such as lungs, heart, muscles, kidney and liver. In addition, a transcript having a size of approximately 2 kb was also expressed in the normal tissues.

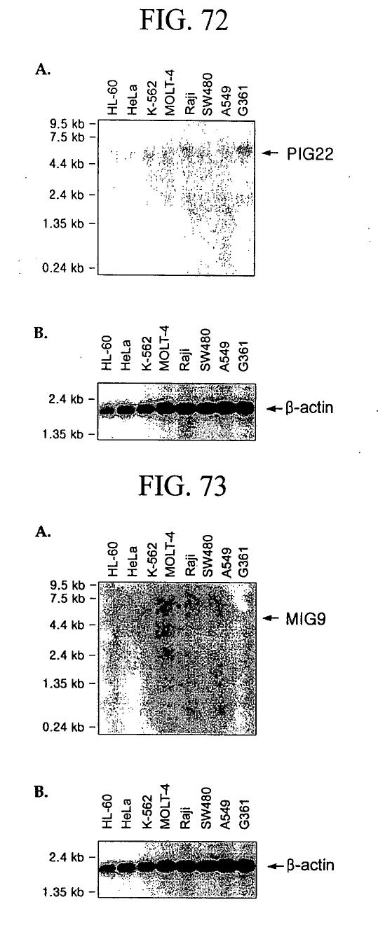

FIG. 72(a) shows a northern blotting result that the PIG22 gene is differentially expressed in various cancer cell lines, and FIG. 72(b) shows a northern blotting result obtained by hybridizing the same blot with β-actin probe. As shown in FIG. 72(a), the approximately 1.3-kb dominant PIG22 mRNA transcript detected in the normal tissues was not at all expressed or slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the PIG22 gene of the present invention had the tumor suppresser function in the normal tissues such as lungs, heart, muscles, liver and placenta.

3-13: MIG9

In order to assess an expression level of the MIG9 gene, the northern blotting was carried out, as follows.

20 μg of each of the total RNA samples obtained from the three normal lung tissues, the two primary lung cancer tissues, the two metastatic lung cancer tissue and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length MIG9 cDNA using the Rediprime II random prime labelling system (Amersham, United Kingdom). The northern blotting procedure was repeated twice; one was quantitified using the densitometer and the other was hybridized with the β-actin probe to determine the total mRNA.

FIG. 43(a) shows the northern blotting result that the MIG9 gene is differentially expressed in a normal lung tissue, a primary lung cancer tissue, a metastatic lung cancer tissue and a lung cancer cell line, and FIG. 43(b) shows the northern blotting result obtained by hybridizing the same blot with β-actin probe. As shown in FIGS. 43(a) and (b), it was revealed that the expression level of the MIG9 gene was highly detected all in the three samples of the normal lung tissue, but its expression level was slightly detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

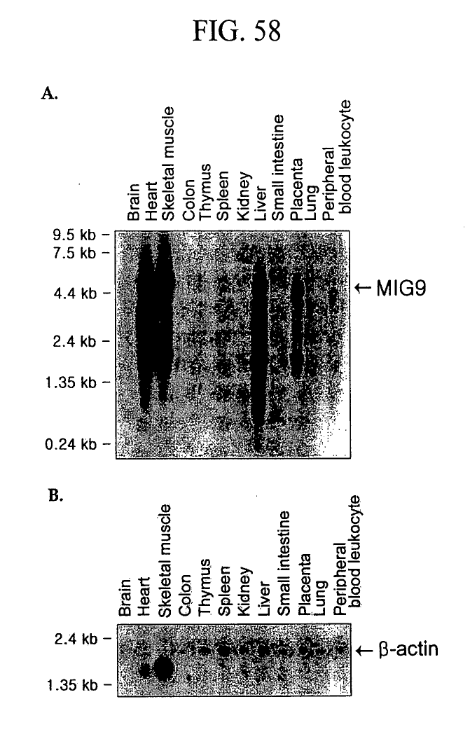

FIG. 58(a) shows a northern blotting result that the MIG9 gene is differentially expressed in various normal tissues, and FIG. 58(b) shows a northern blotting result obtained by hybridizing the same blot with β-actin probe. As shown in FIG. 58(a), a dominant MIG9 mRNA transcript having a size of approximately 5 kb was highly overexpressed in the normal tissues such as heart, muscles, kidney, liver, placenta and peripheral blood. In addition, a transcript having a size of approximately 2 kb was also expressed in the normal tissues.

FIG. 73(a) shows a northern blotting result that the MIG9 gene is differentially expressed in various cancer cell lines, and FIG. 73(b) shows a northern blotting result obtained by hybridizing the same blot with β-actin probe. As shown in FIG. 73(a), the approximately 5-kb dominant MIG9 mRNA transcript detected in the normal tissues was not expressed or slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the MIG9 gene of the present invention had the tumor suppresser function in the normal tissues such as lungs, heart, muscles, kidney, liver, placenta and peripheral blood.

3-14: MIG11

In order to assess an expression level of the MIG11 gene, the northern blotting was carried out, as follows.

20 μg of each of the total RNA samples obtained from the three normal lung tissues, the two primary lung cancer tissues, the two metastatic lung cancer tissue and the lung cancer cell lines (A549 and NCI-H358) as described in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe prepared from the full-length MIG11 cDNA using the Rediprime II random prime labelling system (Amersham, United Kingdom). The northern blotting procedure was repeated twice; one was quantitified using the densitometer and the other was hybridized with the β-actin probe to determine the total mRNA.

FIG. 44(a) shows the northern blotting result that the MIG11 gene is differentially expressed in a normal lung tissue, a primary lung cancer tissue, a metastatic lung cancer tissue and a lung cancer cell line, and FIG. 44(b) shows the northern blotting result obtained by hybridizing the same blot with β-actin probe. As shown in FIGS. 44(a) and (b), it was revealed that the expression level of the MIG11 gene was highly detected all in the three samples of the normal lung tissue, but its expression level was slightly detected in the two samples of the lung cancer tissue, the two samples of the metastatic lung cancer tissue and the two samples of the lung cancer cell line.

The northern blotting was carried out on the normal human multiple tissue (Clontech) and the human cancer cell line (Clontech). That is to say, the northern blotting was carried out by hybridizing blots, into which each of the total RNA samples extracted from the normal tissues and the cancer cell lines was transferred, in the same manner as described above, wherein the blots were commercially available from the company Clontech (U.S.), and the normal tissue is, for example, selected from the group consisting of brain, heart, skeletal muscles, colon, thymus, spleen, kidney, liver, small intestines, placenta, lungs and peripheral blood leukocyte, and the cancer cell line is, for example, selected from the group consisting of promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell.

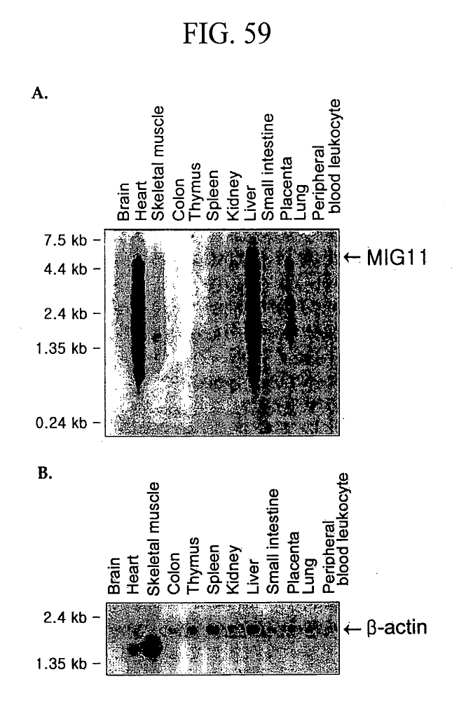

FIG. 59(a) shows a northern blotting result that the MIG11 gene is differentially expressed in various normal tissues, and FIG. 59(b) shows a northern blotting result obtained by hybridizing the same blot with β-actin probe. As shown in FIG. 59(a), a dominant MIG11 mRNA transcript having a size of approximately 5 kb was highly overexpressed in the normal tissues such as heart, muscles, spleen, kidney, liver, placenta and peripheral blood. In addition, a transcript having a size of approximately 2 kb was also expressed in the normal tissues.

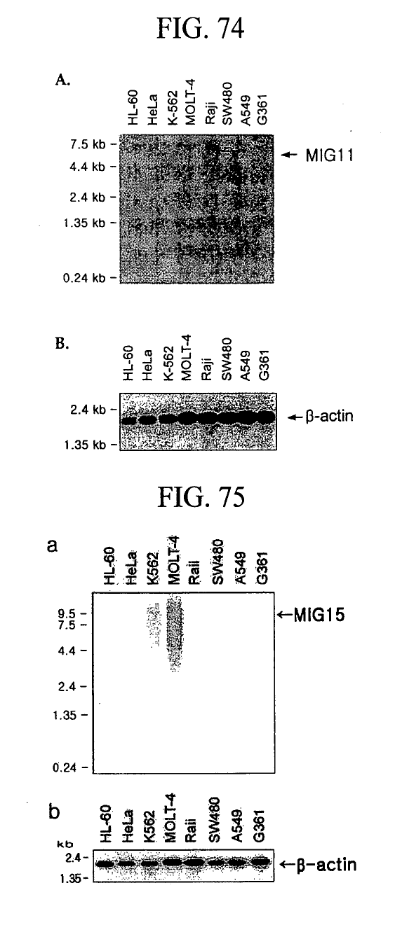

FIG. 74(a) shows a northern blotting result that the MIG11 gene is differentially expressed in various cancer cell lines, and FIG. 74(b) shows a northern blotting result obtained by hybridizing the same blot with β-actin probe. As shown in FIG. 74(a), the approximately 5-kb dominant MIG11 mRNA transcript detected in the normal tissues was not expressed or slightly expressed in the tissues such as promyelocytic leukemia HL-60, HeLa cervical cancer cell, a chronic myelocytic leukemia cell line K-562, lymphoblastoid leukemia MOLT-4, Burkitt's lymphoma (Raji), SW480 colon cancer cell, A549 lung cancer cell and G361 melanoma cell. From such a result, it was revealed that the MIG11 gene of the present invention had the tumor suppresser function in the normal tissues such as lungs, heart, muscles, spleen, kidney, liver, placenta and peripheral blood.

3-15: MIG15

In order to assess an expression level of the MIG15 gene, the northern blotting was carried out, as follows.

20 μg of each of the total RNA samples obtained from the three normal exocervical tissues, the three primary cervical cancer tissues and the two cervical cancer cell line as obtained in Example 1 was denatured and electrophoresized in a 1% formaldehyde agarose gel, and then the resultant agarose gel was transferred to a nylon membrane (Boehringer-Mannheim, Germany). The nylon membrane was then hybridized at 42° C. overnight with the ³²P-labeled random prime probe using the full-length MIG15 cDNA obtained in Example 1. The northern blotting procedure was repeated twice; one was quantitified using the densitometer and the other was hybridized with the β-actin probe to determine the total mRNA.

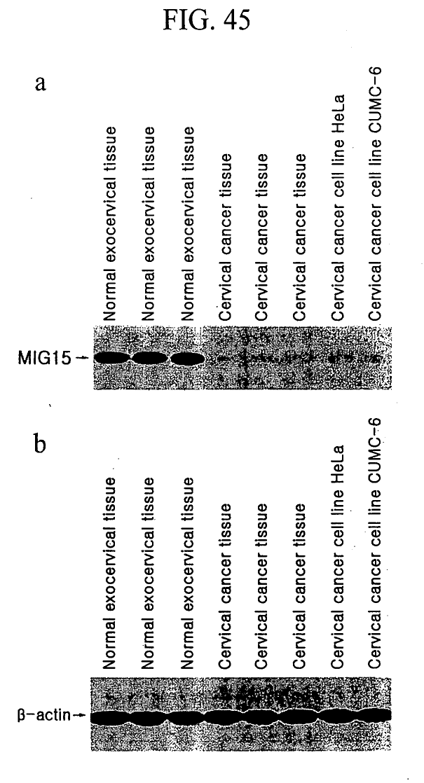

FIG. 45(a) shows the northern blotting result that the MIG15 gene is differentially expressed in a normal exocervical tissue, a primary cervical cancer tissue and a cervical cancer cell line, and FIG. 45(b) is a northern blotting result showing expression of β-actin. In FIGS. 45(a) and (b), Lanes 1 to 3 represent the normal exocervical tissue samples, Lanes 4 to 6 represent the cervical cancer tissue samples, Lane 7 represents the sample of the cervical cancer cell line HeLa, and Lane 8 represents the sample of the cervical cancer cell line CUMC-6. As shown in FIGS. 45(a) and (b), it was revealed that the expression level of the MIG15 gene was highly detected all in the three samples of the normal exocervical tissue, but its expression level was significantly lower in the three samples of the cervical cancer tissue than the normal tissue, and slightly detected in the two samples of the cervical cancer cell line.

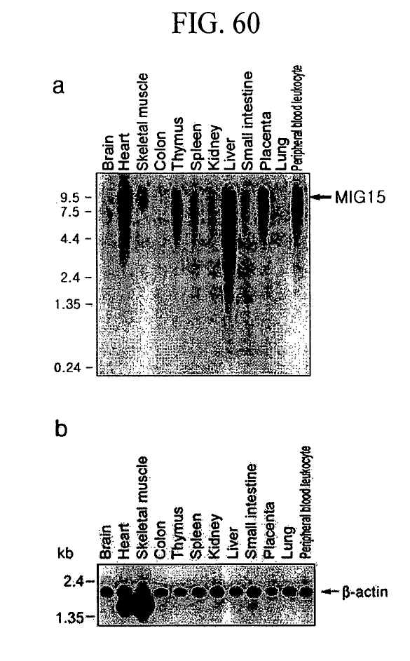

FIG. 60(a) shows a northern blotting result that the MIG15 gene is differentially expressed in various normal tissues, and FIG. 60(b) shows a northern blotting result obtained by hybridizing the same blot with β-actin probe. As shown in FIG. 60(a), a dominant MIG15 mRNA transcript having a size of approximately 9.5 kb was overexpressed in the normal tissues such as heart, skeletal muscles, thymus, spleen, kidney, liver, small intestines, placenta and peripheral blood.

FIG. 75(a) shows a northern blotting result that the MIG15 gene is differentially expressed in various cancer cell lines, and FIG. 75(b) shows a northern blotting result obtained by hybridizing the same blot with β-actin probe. As shown in FIG. 75(a), the MIG15 gene was not expressed in the tissues such as promyelocytic leukemia HL-60, Burkitt's lymphoma (Raji), SW480 colon cancer cell, A549 lung cancer cell and G361 melanoma cell, and very slightly expressed in the tissues such as HeLa cervical cancer cell, a chronic myelocytic leukemia cell line K-562 and lymphoblastoid leukemia MOLT-4.

From such a result, it was revealed that the MIG15 gene of the present invention had the tumor suppresser function in the normal tissues such as cervix, heart, skeletal muscles, thymus, spleen, kidney, liver, small intestines, placenta and peripheral blood.

### Example 4

### Construction and Transfection of Expression Vector

4-1: GIG1

An expression vector containing a coding region of GIG1 was constructed, as follows. At first, the full-length GIG1 cDNA clone prepared in Example 2 was inserted into a eukaryotic expression vector pcDNA3.1 (Invitrogen, U.S.) to obtain an expression vector pcDNA3.1/GIG1. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the GIG1 cDNA was used as the control group.

4-2: GIG3

An expression vector containing a coding region of GIG3 was constructed, as follows. At first, the full-length GIG3 cDNA clone prepared in Example 2 was inserted into a eukaryotic expression vector pcDNA3.1 (Invitrogen, U.S.) to obtain an expression vector pcDNA3.1/GIG3. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the GIG3 cDNA was used as the control group.

4-3: GIG4

An expression vector containing a coding region of GIG4 was constructed, as follows. At first, the full-length GIG4 cDNA clone prepared in Example 2 was inserted into a eukaryotic expression vector pcDNA3.1 (Invitrogen, U.S.) to obtain an expression vector pcDNA3.1/GIG4. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the GIG4 cDNA was used as the control group.

4-4: GIG5

An expression vector containing a coding region of GIG5 was constructed, as follows. At first, the full-length GIG5 cDNA clone prepared in Example 2 was inserted into a eukaryotic expression vector pcDNA3.1 (Invitrogen, U.S.) to obtain an expression vector pcDNA3.1/GIG5. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the GIG5 cDNA was used as the control group.

4-5: GIG1

An expression vector containing a coding region of GIG11 was constructed, as follows. At first, the full-length GIG11 cDNA clone prepared in Example 2 was inserted into a eukaryotic expression vector pcDNA3.1 (Invitrogen, U.S.) to obtain an expression vector pcDNA3.1/GIG11. The expression vector was transfected into an MCF-7 breast cancer cell line using lipofectamine (Gibco BRL), and then incubated in a DMEM medium including 0.6 mg/ml of G418 (Gibco) to select transfected cells. At this time, the MCF-7 cell transfected by the expression vector pcDNA3.1 devoid of the GIG11 cDNA was used as the control group.

4-6: MIG2

An expression vector containing a coding region of MIG2 was constructed, as follows. At first, the full-length MIG2 cDNA clone prepared in Example 2 was inserted into a eukaryotic expression vector pcDNA3.1 (Invitrogen, U.S.) to obtain an expression vector pcDNA3.1/MIG2. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the MIG2 cDNA was used as the control group.

4-7: MIG4

An expression vector containing a coding region of MIG4 was constructed, as follows. At first, the full-length MIG4 cDNA clone prepared in Example 2 was inserted into a eukaryotic expression vector pcDNA3.1 (Invitrogen, U.S.) to obtain an expression vector pcDNA3.1/MIG4. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the MIG4 cDNA was used as the control group.

4-8: PIG13

An expression vector containing a coding region of PIG13 was constructed, as follows. At first, the full-length PIG13 cDNA clone prepared in Example 2 was inserted into a eukaryotic expression vector pcDNA3.1 (Invitrogen, U.S.) to obtain an expression vector pcDNA3.1/PIG13. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the PIG13 cDNA was used as the control group.

4-9: PIG15

An expression vector containing a coding region of PIG15 was constructed, as follows. At first, the full-length PIG15 cDNA clone prepared in Example 2 was inserted into a eukaryotic expression vector pcDNA3.1 (Invitrogen, U.S.) to obtain an expression vector pcDNA3.1/PIG15. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the PIG15 cDNA was used as the control group.

4-10: PIG8

An expression vector containing a coding region of PIG8 was constructed, as follows. At first, the full-length PIG8 cDNA clone prepared in Example 2 was inserted into a eukaryotic expression vector pcDNA3.1 (Invitrogen, U.S.) to obtain an expression vector pcDNA3.1/PIG8. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the PIG8 cDNA was used as the control group.

4-11: MRG1

An expression vector containing a coding region of MRG1 was constructed, as follows. At first, the full-length MRG1 cDNA clone prepared in Example 2 was inserted into a eukaryotic expression vector pcDNA3.1 (Invitrogen, U.S.) to obtain an expression vector pcDNA3.1/MRG1. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the MRG1 cDNA was used as the control group.

4-12: PIG22

An expression vector containing a coding region of PIG22 was constructed, as follows. At first, the full-length PIG22 cDNA clone prepared in Example 2 was inserted into a eukaryotic expression vector pcDNA3.1 (Invitrogen, U.S.) to obtain an expression vector pcDNA3.1/PIG22. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the PIG22 cDNA was used as the control group.

4-13: MIG9

An expression vector containing a coding region of MIG9 was constructed, as follows. At first, the full-length MIG9 cDNA clone prepared in Example 2 was inserted into a eukaryotic expression vector pcDNA3.1 (Invitrogen, U.S.) to obtain an expression vector pcDNA3.1/MIG9. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the MIG9 cDNA was used as the control group.

4-14: MIG11

An expression vector containing a coding region of MIG11 was constructed, as follows. At first, the full-length MIG11 cDNA clone prepared in Example 2 was inserted into a eukaryotic expression vector pcDNA3.1 (Invitrogen, U.S.) to obtain an expression vector pcDNA3.1/MIG11. The expression vector was transfected into an A549 lung cancer cell line using lipofectamine (Gibco BRL), and then incubated in a DMEM medium including 0.6 mg/ml of G418 (Gibco) to select transfected cells. At this time, the A549 cell transfected by the expression vector pcDNA3.1 devoid of the MIG11 cDNA was used as the control group.

4-15: MIG15

An expression vector containing a coding region of MIG15 was constructed, as follows. At first, the full-length MIG15 cDNA clone prepared in Example 2 was inserted into a eukaryotic expression vector pcDNA3.1 (Invitrogen, U.S.) to obtain an expression vector pcDNA3.1/MIG15. The expression vector was transfected into an HeLa cervical cancer cell line (ATCC CCL-2) using lipofectamine (Gibco BRL), and then incubated in a DMEM medium including 0.6 mg/ml of G418 (Gibco) to select transfected cells. At this time, the HeLa cell transfected by the expression vector pcDNA3.1 devoid of the MIG15 cDNA was used as the control group.

### Example 5

### Growth Curve of Cell Transfected by Gene

5-1: GIG1

In order to determine an effect of the GIG1 gene on growth of the cervical cancer cell, the normal HeLa cell, the HeLa cervical cancer cell transfected by the GIG1 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

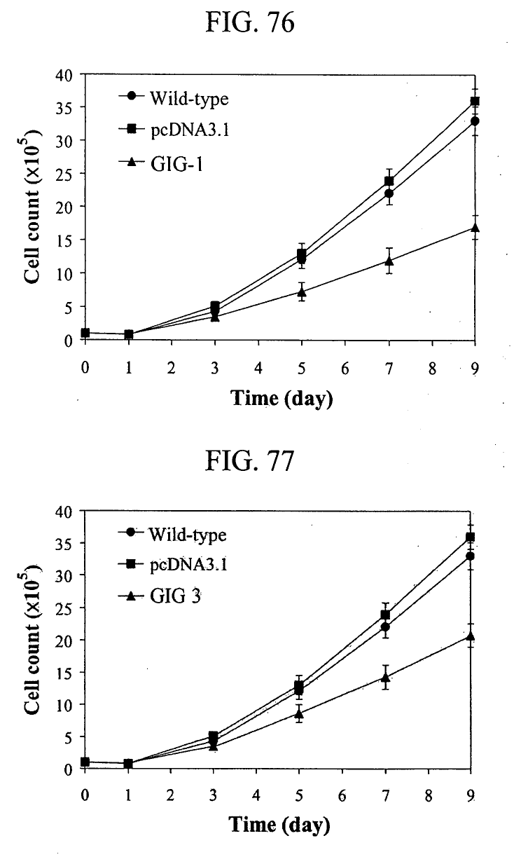

FIG. 76 shows growth curves of the normal HeLa cell, the HeLa cervical cancer cell transfected by the GIG1 gene prepared in Example 4, and the HeLa cell transfected only by the expression vector pcDNA3.1. As shown in FIG. 76, it was revealed that the HeLa cervical cancer cell transfected by the GIG1 gene exhibited a higher mortality, compared to those of the HeLa cell transfected by the expression vector pcDNA3.1 and the normal HeLa cell. After 9 days of incubation, only 50% of the HeLa cervical cancer cell transfected by the GIG1 gene was survived when compared to the normal HeLa cell. From such a result, it might be seen that the GIG1 gene suppressed growth of the cervical cancer cell.

5-2: GIG3

In order to determine an effect of the GIG3 gene on growth of the lung cancer cell, the wild-type A549 cell, the A549 lung cancer cell transfected by the vector pcDNA3.1/GIG3 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 77 shows growth curves of the wild-type A549 cell, the A549 lung cancer cell transfected by the vector pcDNA3.1/GIG3 prepared in Example 4, and the A549 cell transfected only by the expression vector pcDNA3.1. As shown in FIG. 77, it was revealed that the A549 lung cancer cell transfected by the vector pcDNA3.1/GIG3 exhibited a higher mortality, compared to those of the A549 cell transfected by the expression vector pcDNA3.1 and the wild-type A549 cell. After 9 days of incubation, only approximately 70% of the A549 lung cancer cell transfected by the vector pcDNA3.1/GIG3 was survived when compared to the wild-type A549 cell. From such a result, it might be seen that the GIG3 gene suppressed growth of the lung cancer cell.

5-3: GIG4

In order to determine an effect of the GIG4 gene on growth of the lung cancer cell, the wild-type A549 cell, the A549 lung cancer cell transfected by the vector pcDNA3.1/GIG4 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

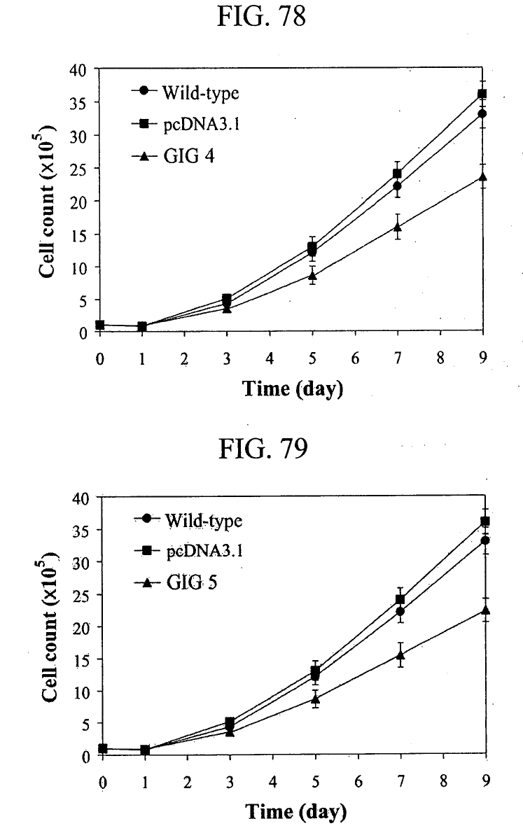

FIG. 78 shows growth curves of the wild-type A549 cell, the A549 lung cancer cell transfected by the vector pcDNA3.1/GIG4 prepared in Example 4, and the A549 cell transfected only by the expression vector pcDNA3.1. As shown in FIG. 78, it was revealed that the A549 lung cancer cell transfected by the vector pcDNA3.1/GIG4 exhibited a higher mortality, compared to those of the A549 cell transfected by the expression vector pcDNA3.1 and the wild-type A549 cell. After 9 days of incubation, only approximately 70% of the A549 lung cancer cell transfected by the vector pcDNA3.1/GIG4 was survived when compared to the wild-type A549 cell. From such a result, it might be seen that the GIG4 gene suppressed growth of the lung cancer cell.

5-4: GIG5

In order to determine an effect of the GIG5 gene on growth of the lung cancer cell, the wild-type A549 cell, the A549 lung cancer cell transfected by the vector pcDNA3.1/GIG5 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 79 shows growth curves of the wild-type A549 cell, the A549 lung cancer cell transfected by the vector pcDNA3.1/GIG5 prepared in Example 4, and the A549 cell transfected only by the expression vector pcDNA3.1. As shown in FIG. 79, it was revealed that the A549 lung cancer cell transfected by the vector pcDNA3.1/GIG5 exhibited a higher mortality, compared to those of the A549 cell transfected by the expression vector pcDNA3.1 and the wild-type A549 cell. After 9 days of incubation, only approximately 70% of the A549 lung cancer cell transfected by the vector pcDNA3.1/GIG5 was survived when compared to the wild-type A549 cell. From such a result, it might be seen that the GIG5 gene suppressed growth of the lung cancer cell.

5-5: GIG11

In order to determine an effect of the GIG11 gene on growth of the breast cancer cell, the wild-type MCF-7 cell, the MCF-7 breast cancer cell transfected by the vector pcDNA3.1/GIG11 prepared in Example 4, and the MCF-7 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

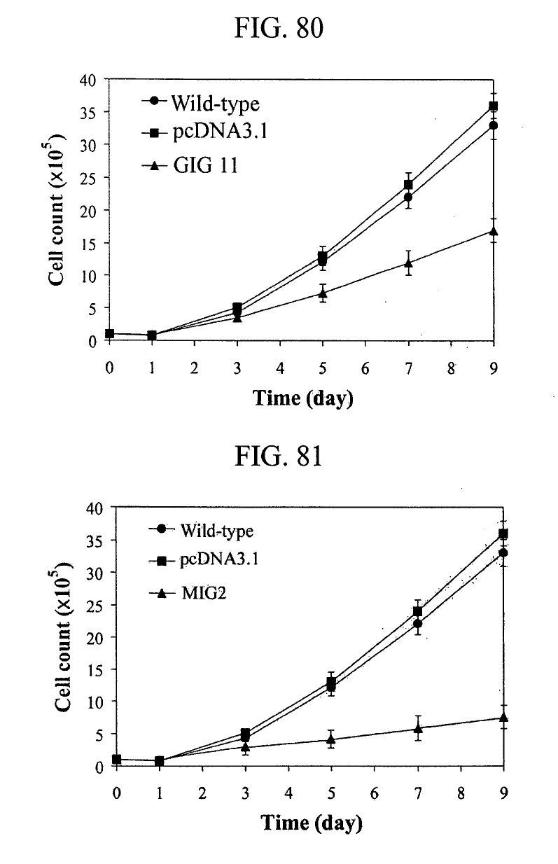

FIG. 80 shows growth curves of the wild-type MCF-7 cell, the MCF-7 breast cancer cell transfected by the vector pcDNA3.1/GIG11 prepared in Example 4, and the MCF-7 cell transfected only by the expression vector pcDNA3.1. As shown in FIG. 80, it was revealed that the MCF-7 breast cancer cell transfected by the vector pcDNA3.1/GIG11 exhibited a higher mortality, compared to those of the MCF-7 cell transfected by the expression vector pcDNA3.1 and the wild-type MCF-7 cell. After 9 days of incubation, only 50% of the MCF-7 breast cancer cell transfected by the vector pcDNA3.1/GIG11 was survived when compared to the wild-type MCF-7 cell. From such a result, it might be seen that the GIG11 gene suppressed growth of the breast cancer cell.

5-6: MIG2

In order to determine an effect of the MIG2 gene on growth of the cervical cancer cell, the normal HeLa cell, the HeLa cervical cancer cell transfected by the MIG2 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 81 shows growth curves of the normal HeLa cell, the HeLa cervical cancer cell transfected by the MIG2 gene prepared in Example 4, and the HeLa cell transfected only by the expression vector pcDNA3.1. As shown in FIG. 81, it was revealed that the HeLa cervical cancer cell transfected by the MIG2 gene exhibited a higher mortality, compared to those of the HeLa cell transfected by the expression vector pcDNA3.1 and the normal HeLa cell. After 9 days of incubation, only 20% of the HeLa cervical cancer cell transfected by the MIG1 gene was survived when compared to the normal HeLa cell. From such a result, it might be seen that the MIG2 gene suppressed growth of the cervical cancer cell.

5-7: MIG4

In order to determine an effect of the MIG4 gene on growth of the cervical cancer cell, the normal HeLa cell, the HeLa cervical cancer cell transfected by the MIG4 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

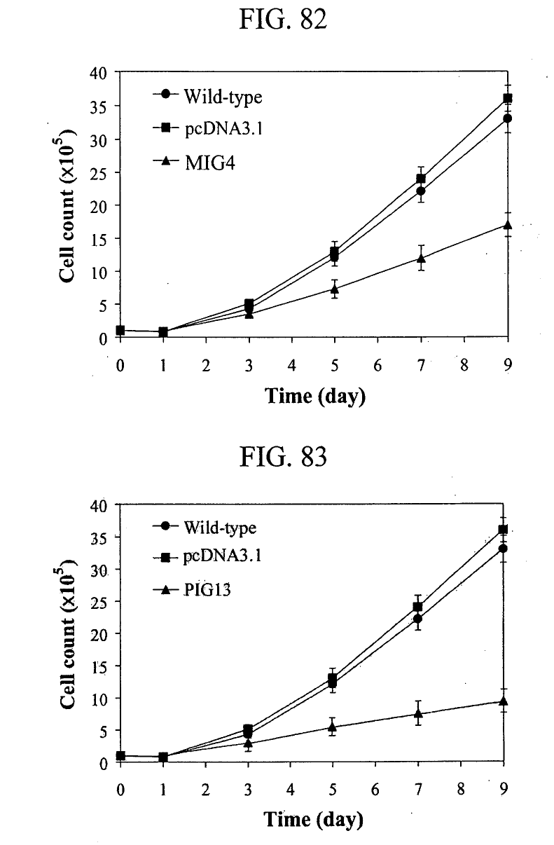

FIG. 82 shows growth curves of the normal HeLa cell, the HeLa cervical cancer cell transfected by the MIG4 gene prepared in Example 4, and the HeLa cell transfected only by the expression vector pcDNA3.1. As shown in FIG. 82, it was revealed that the HeLa cervical cancer cell transfected by the MIG4 gene exhibited a higher mortality, compared to those of the HeLa cell transfected by the expression vector pcDNA3.1 and the normal HeLa cell. After 9 days of incubation, only 50% of the HeLa cervical cancer cell transfected by the MIG4 gene was survived when compared to the normal HeLa cell. From such a result, it might be seen that the MIG4 gene suppressed growth of the cervical cancer cell.

5-8: PIG13

In order to determine an effect of the PIG13 gene on growth of the lung cancer cell, the wild-type A549 cell, the A549 lung cancer cell transfected by the vector pcDNA3.1/PIG13 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 83 shows growth curves of the wild-type A549 cell, the A549 lung cancer cell transfected by the vector pcDNA3.1/PIG13 prepared in Example 4, and the A549 cell transfected only by the expression vector pcDNA3.1. As shown in FIG. 83, it was revealed that the A549 lung cancer cell transfected by the vector pcDNA3.1/PIG13 exhibited a higher mortality, compared to those of the A549 cell transfected by the expression vector pcDNA3.1 and the wild-type A549 cell. After 9 days of incubation, only approximately 30% of the A549 lung cancer cell transfected by the vector pcDNA3.1/PIG13 was survived when compared to the wild-type A549 cell. From such a result, it might be seen that the PIG13 gene suppressed growth of the lung cancer cell.

5-9: PIG15

In order to determine an effect of the PIG15 gene on growth of the lung cancer cell, the wild-type A549 cell, the A549 lung cancer cell transfected by the vector pcDNA3.1/PIG15 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

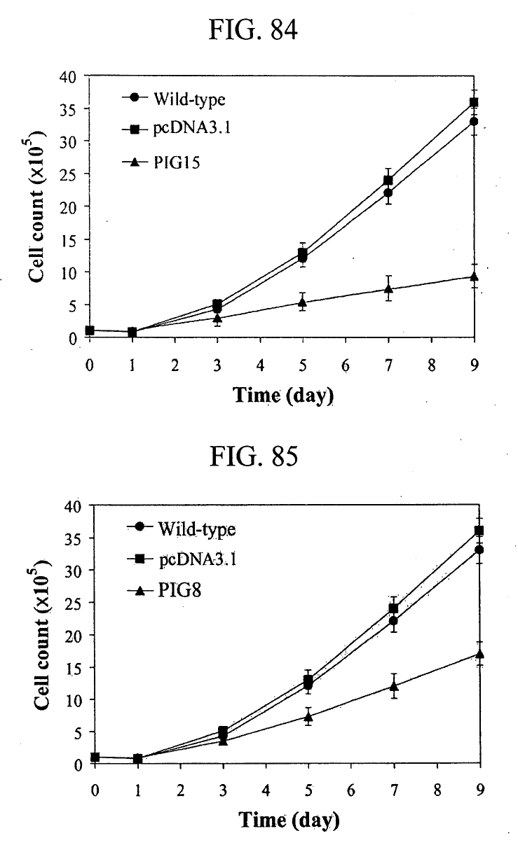

FIG. 84 shows growth curves of the wild-type A549 cell, the A549 lung cancer cell transfected by the vector pcDNA3.1/PIG15 prepared in Example 4, and the A549 cell transfected only by the expression vector pcDNA3.1. As shown in FIG. 84, it was revealed that the A549 lung cancer cell transfected by the vector pcDNA3.1/PIG15 exhibited a higher mortality, compared to those of the A549 cell transfected by the expression vector pcDNA3.1 and the wild-type A549 cell. After 9 days of incubation, only approximately 30% of the A549 lung cancer cell transfected by the vector pcDNA3.1/PIG15 was survived when compared to the wild-type A549 cell. From such a result, it might be seen that the PIG15 gene suppressed growth of the lung cancer cell.

5-10: PIG 8

In order to determine an effect of the PIG8 gene on growth of the cervical cancer cell, the normal HeLa cell, the HeLa cervical cancer cell transfected by the PIG8 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 85 shows growth curves of the normal HeLa cell, the HeLa cervical cancer cell transfected by the PIG8 gene prepared in Example 4, and the HeLa cell transfected only by the expression vector pcDNA3.1. As shown in FIG. 85, it was revealed that the HeLa cervical cancer cell transfected by the PIG8 gene exhibited a higher mortality, compared to those of the HeLa cell transfected by the expression vector pcDNA3.1 and the normal HeLa cell. After 9 days of incubation, only 50% of the HeLa cervical cancer cell transfected by the PIG8 gene was survived when compared to the normal HeLa cell. From such a result, it might be seen that the PIG8 gene suppressed growth of the cervical cancer cell.

5-11: MRG1

In order to determine an effect of the MRG1 gene on growth of the cervical cancer cell, the normal HeLa cell, the HeLa cervical cancer cell transfected by the MRG1 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

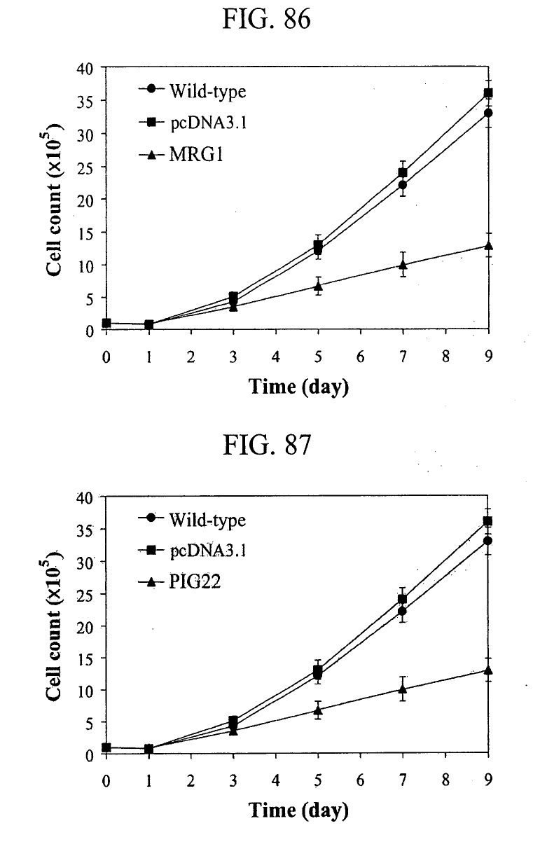

FIG. 86 shows growth curves of the normal HeLa cell, the HeLa cervical cancer cell transfected by the MRG1 gene prepared in Example 4, and the HeLa cell transfected only by the expression vector pcDNA3.1. As shown in FIG. 86, it was revealed that the HeLa cervical cancer cell transfected by the MRG1 gene exhibited a higher mortality, compared to those of the HeLa cell transfected by the expression vector pcDNA3.1 and the normal HeLa cell. After 9 days of incubation, only 40% of the HeLa cervical cancer cell transfected by the MRG1 gene was survived when compared to the normal HeLa cell. From such a result, it might be seen that the MRG1 gene suppressed growth of the cervical cancer cell.

5-12: PIG22

In order to determine an effect of the PIG22 gene on growth of the lung cancer cell, the wild-type A549 cell, the A549 lung cancer cell transfected by the vector pcDNA3.1/PIG22 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 87 shows growth curves of the wild-type A549 cell, the A549 lung cancer cell transfected by the vector pcDNA3.1/PIG22 prepared in Example 4, and the A549 cell transfected only by the expression vector pcDNA3.1. As shown in FIG. 87, it was revealed that the A549 lung cancer cell transfected by the vector pcDNA3.1/PIG22 exhibited a higher mortality, compared to those of the A549 cell transfected by the expression vector pcDNA3.1 and the wild-type A549 cell. After 9 days of incubation, only approximately 40% of the A549 lung cancer cell transfected by the vector pcDNA3.1/PIG22 was survived when compared to the wild-type A549 cell. From such a result, it might be seen that the PIG22 gene suppressed growth of the lung cancer cell.

5-13: MIG9

In order to determine an effect of the MIG9 gene on growth of the lung cancer cell, the wild-type A549 cell, the A549 lung cancer cell transfected by the vector pcDNA3.1/MIG9 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

FIG. 88 shows growth curves of the wild-type A549 cell, the A549 lung cancer cell transfected by the vector pcDNA3.1/MIG9 prepared in Example 4, and the A549 cell transfected only by the expression vector pcDNA3.1. As shown in FIG. 88, it was revealed that the A549 lung cancer cell transfected by the vector pcDNA3.1/MIG9 exhibited a higher mortality, compared to those of the A549 cell transfected by the expression vector pcDNA3.1 and the wild-type A549 cell. After 9 days of incubation, only approximately 40% of the A549 lung cancer cell transfected by the vector pcDNA3.1/MIG9 was survived when compared to the wild-type A549 cell. From such a result, it might be seen that the MIG9 gene suppressed growth of the lung cancer cell.

5-14: MIG11

In order to determine an effect of the MIG11 gene on growth of the lung cancer cell, the wild-type A549 cell, the A549 lung cancer cell transfected by the vector pcDNA3.1/MIG11 prepared in Example 4, and the A549 cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

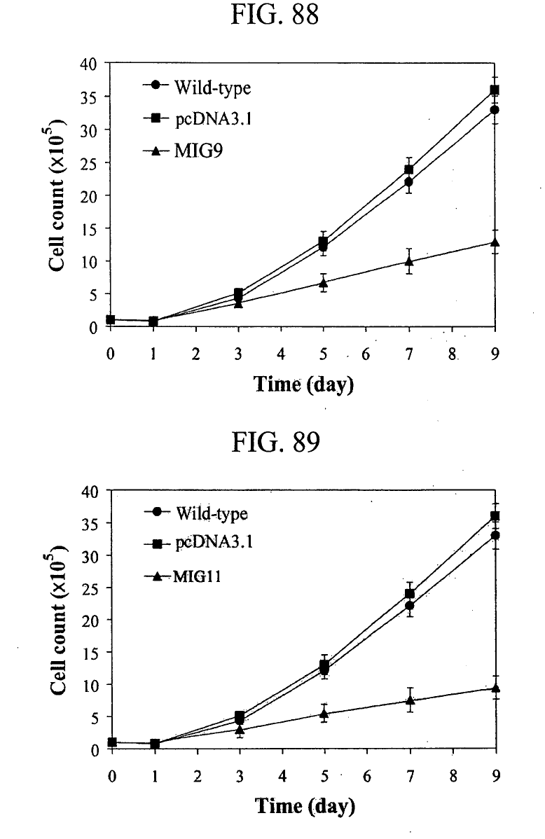

FIG. 89 shows growth curves of the wild-type A549 cell, the A549 lung cancer cell transfected by the vector pcDNA3.1/MIG11 prepared in Example 4, and the A549 cell transfected only by the expression vector pcDNA3.1. As shown in FIG. 89, it was revealed that the A549 lung cancer cell transfected by the vector pcDNA3.1/MIG11 exhibited a higher mortality, compared to those of the A549 cell transfected by the expression vector pcDNA3.1 and the wild-type A549 cell. After 9 days of incubation, only approximately 30% of the A549 lung cancer cell transfected by the vector pcDNA3.1/MIG11 was survived when compared to the wild-type A549 cell. From such a result, it might be seen that the MIG11 gene suppressed growth of the lung cancer cell.

5-15: MIG15

In order to determine an effect of the MIG15 gene on growth of the cervical cancer cell, the normal HeLa cell, the HeLa cervical cancer cell transfected by the MIG15 gene prepared in Example 4, and the HeLa cell transfected only by the vector pcDNA3.1 were incubated at a cell density of 1×10⁵ cells/ml in a DMEM medium for 9 days, respectively. The cells were isolated from the flask they attach to in each of the culture solutions by treatment with trypsin (Sigma), and then the survived cells were counted on days 1, 3, 5, 7 and 9 according to a trypan blue dye exclusion (Freshney, I. R., Culture of Animal Cells, 2nd Ed. A. R. Liss, New York (1987)).

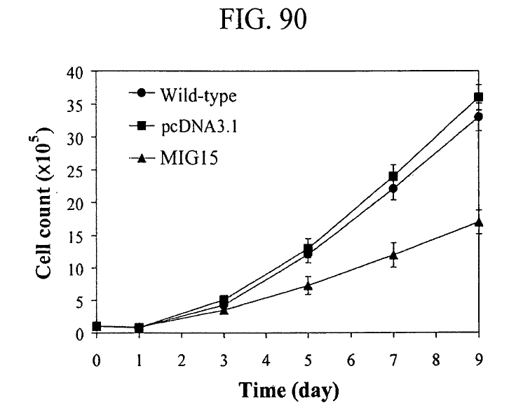

FIG. 90 shows growth curves of the normal HeLa cell, the HeLa cervical cancer cell transfected by the MIG15 gene prepared in Example 4, and the HeLa cell transfected only by the expression vector pcDNA3.1. As shown in FIG. 90, it was revealed that the HeLa cervical cancer cell transfected by the MIG15 gene exhibited a higher mortality, compared to those of the HeLa cell transfected by the expression vector pcDNA3.1 and the normal HeLa cell. After 9 days of incubation, only approximately 50% of the HeLa cervical cancer cell transfected by the MIG15 gene was survived when compared to the normal HeLa cell. From such a result, it might be seen that the MIG15 gene suppressed growth of the cervical cancer cell.

### INDUSTRIAL APPLICABILITY

As seen above, the genes of the present invention may be useful to diagnose and prevent the human cancers.

What is claimed is:

1. A human cancer suppressor protein having an amino acid sequence selected from the group consisting of SEQ ID NO: 2; SEQ ID NO: 6; SEQ ID NO: 10; SEQ ID NO: 14; SEQ ID NO: 18; SEQ ID NO: 22; SEQ ID NO: 26; SEQ ID NO: 30; SEQ ID NO: 34; SEQ ID NO: 38; SEQ ID NO: 42; SEQ ID NO: 46; SEQ ID NO: 50; SEQ ID NO: 54; and SEQ ID NO: 58.

2. The human cancer suppressor gene according to claim 1, wherein a human cancer suppressor gene is defined in a DNA sequence selected from the group consisting of SEQ ID NO: 1; SEQ ID NO: 5; SEQ ID NO: 9; SEQ ID NO: 13; SEQ ID NO: 17; SEQ ID NO: 21; SEQ ID NO: 25; SEQ ID NO: 29; SEQ ID NO: 33; SEQ ID NO: 37; SEQ ID NO: 41; SEQ ID NO: 45; SEQ ID NO: 49; SEQ ID NO: 53; and SEQ ID NO: 57, each encoding the proteins.

3. The human cancer suppressor protein according to claim 1, wherein the cancer is derived from a normal tissue selected from the group consisting of lungs, heart, muscles, kidney, uterus, breast and liver.

4. The human cancer suppressor gene according to claim 2, wherein the cancer is derived from a normal tissue selected from the group consisting of lungs, heart, muscles, kidney, uterus, breast and liver.

5. An expression vector containing each of the genes as defined in claim 2.
